SEQUENCE LISTING (11 sequences)

SEQ ID NO: 1 - DNA, 1301 bp, Oryza sativa
acaggcttcacgtagaaccaaaagtggttgcttgcaaggccgagatgcatagtaaattaagcagcagtaactgtagaaatgaacactggatgtctcgttatcgatttctcgatctctccacgaacaaagacgaggagaggtcccctcctgcatggaacaaaccaaaaaagagagagagagagagagagaaagaaacagatatctagaacgaaagtgagaggaagagaatcagatcgatgtataccggtatgtacccaaaaaaaaaatcctagatattgtttgttttcctctgttgttgataatgtgccgttttgactgactcctgttgctgcagatccgagaaacggacgcgaaatcctcttcggtttttgtatactcatccactggtagctgttcatccgccgctgcagagggcaagtcaacaaggccacacaccccaacccccacttgcagctccagtgcgggccccacaaaaccacacgagcggcaagacggtgggcccaccgcttttcgatagtgggcccacctggttttgtttgcgtgggcgtattccatctccgtcgtcctgcatgcaccgcccatgccgccctgggcccaccgctcagcgagagagttctgcgcctttcgtcctggaccgcgcccacggactcgctggtcggcgccgcatctgcacacgtggcgatcttccattggctggggcgtcggagtcggctgactcttcctgcttggcgtgagtgcgtcacttttttcggttcttttcccaaatggtcttggctctcccacacggtcacgcagtttcactgacgtgtgggtcccacttctggtggggacacgtgtcagcctcaccagtgcttggacgcgtgaggaggtcgccttatataggaaggctttgaagccatggagagagtgggtaagtggtaactggcgacgcggtggtgccgtggtggtggtggtgcagcttctctcctcttctgaatcttctcgacttctcgtcctccctgttgcagaggcttctccggcgaggccgcgaggcttgatttcaggtgagcaagcggctgtgatttgtgtttttcgccttttgcttttgagttttcttctgccctgttaaatcccttctcttgatgtgtgtgaggagctgaactgtaattcagtcagttcagatatgtttgtgatttataggtttctgacccatgccgtgatgtttgttctgcttgagaatacttggaagttcagaaccatcctcagaattcagaaagggctttgctcttttcgctagatgcttctttaagcgtcgattcgtc

SEQ ID NO: 2 - DNA, 1302 bp, Oryza sativa
ttggtttttgactgcctcatcaatggagctctctagtctctatagctgatagcttagctcactctttcattcattcatcggtatatacagctcaaaaggcatggctaggccttgtttagtttcccctatttctaactttccatcatatcatattacatcaaaaactttcctacacatataaatttctaacttttttttcaaactctcaactttcttcaaatttccaactttttttttaggaattaaacacactcttacttagcatgagagaatgacagtgaaccatccgtgacattctttgcaaggaggacggacgatgactctgtgaacaggagtctgatctgctctgaactcccaaccgtcccaaccgacgccggccatcacgggcgtgttcgtgccgggcttcctctgctcttgccggactccggctgtcgtcagagctaccaacgagacgtacaaggaaacgcgcgccgcgtcgtgccgtgctagaatggccgtgcacggcactaacgcggctcacgggttttcttttataacacaaaagacgcacatgtgcaaatattactacacacccataacctttttagttacaagtgctgaaaataatggtatgacaatgatgagaaaaaatatgttttacgcaaattttgtactgcatgtgtttcagaatataacaacttagatcggatgatacttaaccgtaatagatgttaagtctcatctgatctttgatcttaagctgttatattttggaatagagtgagtatgttttatgttcttatggtgtctagaaaatgagttagaataggtgagactagaaaacgaaaacgaatggagagatagaatgaaatgggggtaagaagaatcgatggttcagattgaatattaccctctaagaggggagaaatcatatcccaaacccattttctaaacttgaaccatatggtaaaaaaaaactaaacgtaaaaaaataatagaaagtgtaaattaattctaaaaattctaaaatttaaatttagaagaggaccgcagtaaagttaaaaaaagagaaaaaaatctagttgaggaggaaatgcatgagcaagtttaccattaccaccccggctgggctctgggctgtcgtccgcctcagcgtcttaaatgcaacatctaactctcaccaccctcctcttcaatgcccccgtctccctctcgcctataaaccacgccgcccgccaccgccgctcgccatcgccaccgtcgttcacaccacaccacctgctgcacacagctcgatcgagttagcccactagctaccaacca

SEQ ID NO: 3 - DNA, 1305 bp, Oryza sativa
tcaacaaaaaccatccactccatcactgacatgttggccaaaaccattgtactcagctcattcatctgatgcttccaataaagaaaaggagaaaaaaagagcaaagaacacatggaagttgttagctacagactacacacatacagctggagatttcgatgcaaatacaattaattaattagccatgagcaaaacaaaaaagaaaagtgcataagcataactatactgaattgtttaatggcatggccgatcgcgatttggttgttggcacgtccagataatagcaccaattagtgtgatggcgacggtgtccacggtgttggttggtgcaccgagctcaagcagctttctttgcctcttccgtcctaattaagctgcatctcgcgtgcatcatcgctggttgattagcgccaggactaaactaaacagcctcgttctctctctctctctctctctctctctctctctctctctcctgattcttctgctcttctttttattccctgaagtgtcggctgcatgtgtggcttggcctctgtctaatcaaagtcatcagcctctgtcactattttaacaaagctagtaaagctggtcgagtgtttcccgttttgccccttatgactggctattgtagtactagtcgactattatcacttgcaggctgcagctaggtgaaagtgagagtgtaacaaacctttttgtttggaatgattgcctcggatcaggacttcaggagacattgttaacataaaaaacacactataccattaagtaaacgcaacactgaataatggagtgattgatcaattttgttttgaatactaactgtttttttaagttctttgcattctaaacttgcacgaacagataagagacatgcagttttatatgtagcgagttttctaagaacggtataaacgcatacgcttacaactcacgtgcactcatagaaaaaaaaagacacacatgcatactctaccctataaaacacttctgaaaaattaacgaaatcaccattgatgtctaccaccgaaaaatagttaaccataaatacaaataccaacgtcaagttcagaatttaaatattagttaaccgtaacagccgaatcacactatcaaacacagacatacattcccataaaattttccgattaacctctcaatcagttgcggaggggagttgattttcaggtcgtccctgtttgtctgaactctggaatcgccaatgcagcacatcaacagccgtgtgagcgaggggcatccccgtaaaatcacccccagatgttagtgcgtccacatccatct

SEQ ID NO: 4 - DNA, 1409 bp, Oryza sativa
tacccgcgataatcccgtaaaacttagtataaatcaaaatcaatggtttaaatcgttgttaatagtaataatagaattaataagtaaagcctctggatcatgcctgggcacaggcaatgcacgaatttgggaaaaacctcgtgtcaaagttggtgtgttctagactacgattaattgggtacgctggtcgcgcctttgattgtgcagctggtaattattccagaattccacggagacttttgtaacaaacatgtggttaaggcaaactaattcttaggtagtagtacaagcaaaacaattaagctactactacttccagatgatttgtttgttattagttgaccagacagggtacggtcaagctagcgttgcttccactgttgcggtataagtcgtcgtatactagtatactcctacaacggtgcgtgcacaggatcagagtaccgaactgggggtgttctcggtcttgccagctccataccgtgcctttgctatcgcctgacaaaaagctacgcgaaccactcctctctgttcgattccctcccttgtatgcttcttttttttttcccttatcttttacggggaggagacacgtcgactcgtgaatatatcctgaagagcatacttgctgtgtgcgccacacacatgtagagttgtcagttgtacacatgaacccgaccggtcacgggacggcaacacgtccacgcacacgagtacacgacacaaagtaatacgcgagatgcacgatactctgcttacctcccacggcaagtgctaaatacctttccccccacaacccaacctaagcaaagcatactctagtccctctctagtcttccacttttccgcagccagtaaacgcgcccgcgacttttaccacttacctcccccgccttttgcatcaaaagctacaacaacaacaaaaaaaaaaagcgatcgagcgatcgatcgtgctagtgcggtttaccccgacccgcgttcgcgtgccgccaccatctcaaaccgggcatttttcgtcgcccttttcccccgccatcccgcacgcgaccgcccgcgcgctgcgctaacgtgcccttagcccgcggtctggtcggcggtggagccccgccaaccagcgtgacagccgcggaccgcgagccgtgggcgccaaccccccaccccacgcacgcggaaagccgagcaccgaaccgagtcaccgactcgcctcgcgcgcgttcccacccccgcgcccgcgaccaacgcgaggcgagcgagacgcgtcatctcgccccttgccgtgccggagacgggtcgatcgatcagtcggtctacggctctgcgcttggggaaaaccacacaaaaaaacgcgtgacggcgaccgcccgcgaaaccggcggcccaggcccagcctgtcctccatccacccaaac

SEQ ID NO: 5 - DNA, 1170 bp, Oryza sativa
tcactggctggctgaaggctaaggagatggggggaacaaacaaggtacatcggcggctgacggtgttaacatagtggacgacggcactgatggcaggaagtagtaactcgagcgaaattcaacagaggagtagaaggatactgagggaaattcagccgctcgcttaatttgaatttgaatccaaaacgacctacaatctgtaaaattacttatatacgctaatgtgaatcaatctcaagttggggtgcaataaagggtacgcgtagcaattggcggtgcagggcaacccccggtggcctagtccttatcatagacacgtgtctcgtgacgtggctccagcctttgccgcctcccctttctctcgcctccctccctccctcctctctccgcgcccttctctctctcccctctctcgctgctcattggagatcaatctgctttcacgtctcctctctctctccgcgtgcttcctccgagggtctcctcctccaccgccgtctcgcttcccggcacgcgcgcgcgggcggcggcgagctccctctccttcgccactcctccaacccctgtgcggctactccggtgcggagccggcgagcgagcatgttctaagtggctctcttccttaaatctctctacaagaggcaattgaattgaaggtatttgttctcttcctagccgctgctttcatgagcgataggagatccatgctagcaagttggattgatttgttcactccgtccaaattattagtccgtgtcctcctctttgttccggatttcgatgatgcgttggtggtggagttctaattgcagtttgtatcgattgggtgtgtgactgaatatctagttactgaagcagtttcatgttgatgtatgcgtttgccctgtcctgttcctggaaccaggattaataataaacatatgaaaagttatttgacctcgaaaaaaaaattgtgctctccaatgtaggagaccgacgaaacttttgtttttaatagctgcttttagttctaaaaatcataattcgacttgtatgataggaatttgcatcttatgaaaagattgagaattgtttttacacaagttgatagctttatatcgatactgttgggcagtgtgccaaaacgtccttttggtgaaatcaaacatttctcacatttcaggctcacgc

SEQ ID NO: 6 - DNA, 1303 bp, Oryza sativa
taccacgttcctcagtccagttcctcgatcccatcgacctagaaactttgttactataatccataccatccaaatacataaaaccaattatggtcacattttccatcttcttccccttttttttccttcttctcccctctctcccttcccctcatgttgctcagccacgacagcgatgccaaccttcatcgccaccccacatctccaacctctcatcgacccaaaaactttgttaccatgatccataccatctaaatacataaagccaattatggtcacattttccatcttcttcccaattcttttttcttcttctcccctctctcccttcccctcatgttggtctgccacggcaacggtgccaacctccatcgccacccccacatctccaacctctctctctctcctgtggagcagatgacgatagtgcttgccaactcaggcgatactaggtgcctcatgcacatagctctgtccgccaattttgatggtagccccaaggaaacggtcttgagtcgccaaatctccggcgccgggcttgggtctctcatcctcccaccggcagtcgtgaagacgacgacacgatgaggacgagtggaacttgtgaagacgaggagggtgtcaacctagatctggcgagaaccgtcttcttcggcaccgggagcaagcggggccattgggaggagcttgggacgggtggcagcttagcggcgagctcgagtgggggcacatcgagtttcctccttccttctcgaaccaaggtgaatagtaccaacgcttcaccggtgcgttggacaccacttctccattcgcctccctcatcgtgtcctactcgcctcctgacacttttggttcagcgaggtggagagagatatgaagaaagaagagtgatggatagagaaagaagatggaactgacatgcgggtctcacgtctagcgtattttggtctatatgatagaaaacgtaacgtcgatagcatggttgttattttgtacgtattcgtatagcatggatacgaatatatgaattgtaattatatttttttaaatagtaatggtatgtatcgagaaaacgtctagaggtgtttcagctagctccacatgatgatgagctagccgacttgaattcgaagcctcactcgtataatcatttgatattagctcattctctaatattcacatcgtagtggtatgtatccaataaattttttaaatcggttttgtttagcatattcgaaagtaaagccagctgcaggttgttttaagataagattagcacacacactaatccaattccaaccca

SEQ ID NO: 7 - DNA, 1304 bp, Oryza sativa
cagtacgtcgcctatcaccataacgcatccgacgcgagtgctgcctgtcccagctacttgtgtcctagctgcctgtgtcccagggtcccggttacttgtgtcgtacccacccacgctgccagcaaccacatgtaggtgtagaaccaactaaccaaaatgtgtttaattagctatattctattgaaaaactagggtagtggccggggcaaaaattttttagtcactccagtgatcaagatttaaaatttaaattccttaggttctgttgccaattttttttggggaatgttgccaaaaatatttcttcaaacggagagagtaatattatatatattgacgctggatgatgcactgcttgtgttgctggtgtgcaagcttccctgccaagtgccaaacggaatcaaatcctctcatgtgaacttccacttgacgtggtcactgacaggtgggcccgtgagcggtggagcccgcatgtcagtcaccacgttcctctgacttcacgtcagaggattctaatccgtgccaaactgccaacttgtggtcggtgcctgcaagcatcccgcgtgagtgctgcctaccctcgccaaagcccgcgtcttcaacacagccgcgtggatttttttatttttttagacttttttatgctttattattagaccattcaaaaaagtatttccgaaaactgaaccttttggccacgtctgcagtggtgacgtgataagaggctggcgtggcagggcaacgctaccacaccagctacccgtggtgtgatagagttatttggtcaccaggctggataaaaggttcatatttaaaattttttttggaaatcgtcaattaataaaattaaaacataaaaaagttactaccattctgcggtaaaaaagttactgcacttccctgacaaaaatatcaaatatctacaattcgttttttgaccggtggagtggcgcaaatgtagatcactcctacaaattaaataatctgaacggataagataagaaattttaattttaactcgtgatgacagtttactctttaaatgagagtaattaactttctactctaaatttctgaatgggatgcggagaacgggaaggaaaaccagagaataactttttctttcttttttgagaagaacggcagtacggtataggaaagggaataataataacaatatatgttttcgtgtagttatatttaacgtcatttataaacatatactagatactagtaataacggaatatatgtcaataatacaagacgtgaaaaccaaaaatgacgaatggagtgatcgagtagc

SEQ ID NO: 8 - DNA, 1244 bp, Oryza sativa
aagcaagtccgaagaccattactcctcttactcctctcctctgtttcatattataaatcgtttaacctttttcctagttaaagttcgctatgtttgactaaatttataaaataaattagtaacatctataacaccaaattagttttattaaattcaacattgaatatattttgataatatgtttgttttgtattgaaagattattatatttttctacaaatttggtaaaacttaaagatgtttgattaagaacataaatcaaacgacttataatacgaaacggacgaagtagctagctagcaggctatatattgatctactagctacctctgtttttttagtaatcgacagtaaataatattgaataaacacacaaaatttaaccactaatttttttccttcagaatttataaaaacatgtgattctgcaccactgcattaaaataaaattcaaaatgattctaacagtatgtgctttagtgtacaaaatagaataaattagtgttcataaaagtttggataggagaatcctctgttttcaaatagtcagtgatttataattaatgcaagtgttaatatttgcattgtgaaaaatatcattttgtttgttgtagaaacaatacaatgatgcatgcaatattaaggacaatttaaatgcaggagtaaaatttttattaaagacaaagttatatgaaggaaaataaaatggtaggggatcaaaaaccatttttatgccattatatttttaataactttaaggaatacacagtattaaaaaataattatcaaatgttattgtttcccatgttagatatgtctagaacgactactaagatagtattaaatacagagatataaaggaagtctccaaactttttgtttgaattacaaacccaagggctaaagtttattacctgcatgttaattgaaggaaaaatatctgcctcgtactgcactgataacgtacagtggattgagaaaatattaattttggattcatctactagatttactagctcttacaagaggattttcagtaataagttagcactggtacttaactagcgaataacttcgcaaaattctgctagtttaatttattttcaaacaaggattctctctccaacttgtcaacaaaatagcactatatagctgacgaactcttacgttacaacattatagtcgcagggtcatcttcttcttttcccctttcgaaggcattgtcactttgtcagccttctgct

SEQ ID NO: 9 - DNA, 1301 bp, Oryza sativa
cggagatcaaacaggaaacaatctcaccatcacaaccgacctagcaggcgattacagcgatggaatcacgcttaccgcgagcgaatcggatcaaaacggaggggctcgagcggagggagggaatgcttcttcggttcgtctcgtctcgtcgcatcgcgttcgcctctggagttttggattctaccgctctagaagcttccggacgagccccgaatcggcacgggagagcgcacacgcgtggcggcgcctgaaggccttttatgatggggacgcgtggcgtgttggccgtcggatcgtgacacgtgtccacgagatgacgcggttggacggtgcgagtggcgacgtcgttgtgtgggccgcgaagtttgtcatcggctcgtgggcctagccttttctcggccctgaaaggtaggaaaccgaggcctttccatccattttcttctctttttcctgttctattattgatgttgtattgggtgattttgaaacccttttgaaagaaattttgggtaatttttgttcattgaattcttgaatttgatctcctcctaagtaagttgaataaatggatttgtggattgtgagaagctgacctaagacaaaagattttttgcagtagtagtacattaaaaaagcaggcattgcattccatccctcttcaagggctaaggcttatctacctaacattttttcttgtttcctgcacacgatgataacatttcctttctgttgatctgtcattcgtgatttgagtagctatatgtaaataggtttgatgttgtttgacaatatcaccagcatagctattctcatgcttacagttcatctaaccagctttcctgatcagggttcagaggttatatccaaagttttattcagaagaacgcaaaaggaacattcaagattagcatccataatatttatgagtagtacgactagtacattaaacaagctctagattaggatcaacacaagtgatcaaagtgatggggatgagaaagccctggaatggtccgaacccaatgtgtgtaatccaccgattggccttagctgattgtgactccgttcatgtttgatgtgctagtaccaaccacctgtgccactatctgtcctccacatgtacactccataggcgtatggacgtatggaacaaaagataagatacaaagatgcatggagaagattatagttaaaactgaaagctgtacatcactatcatttggtaattcagaattcagaagtgaatatggacatggaatctgcagtactacttgtgcggtagcagtcactgacaagtgggt

SEQ ID NO: 10 - DNA, 1301 bp, Oryza sativa
gatgcaggagtgcgattaagttgcgtcagcggcaagggattattatccctttctatatccggcggcggcgcgctcccggccggttggaggcggccatggacagcctgctacgccacgaaagcatcattatatctgcccagatgattcatacacatatatgatctcaaaaggcgatcgatgtgcgcacgtacatacaggtcacaactggtcatgcctacgatagatatgccgtgcaacatgcgttgctcggagcacttcaattaagatagagggaacaaatgttataagaaagattttcatcggtagtcgactgttgagaaaagatgtgcattctatgtataaacacaagctactgaactgaaagccgagtgccaaaccggttattggtattattcatgatcattcccagattattctgttttaaccagtcgtgggaactattggccaactatgctggatcctatgactcctccctggtcctgagtttcttggaagccttatctacaggttcaatgcacgcacgcacgattgacgacgacctgaacgactcttccggtcaataatgccagcccatgtggtcctccgcagtccgcactgtgcaagtgtgcagcgtccacttgaaaatccacagaagccaccattcttcagagcctccctccctgcttcttcaatacacttccagaactaggggcatgttcgatttagcctggtgtagttggagaaaatttttaattttagttatcatatcggatatacagatatatatttggagtattaaatgtagtctaataacaaaacaaattacagattccgtcagaaaactacaagacgaatttattaagtctaattaatccatcattagcaaatgtttactgtagcactatattgtcaaattatggcataattaggcttaaacgattcgtctcacaatttacacgtaatctgtgtaattggttttttttcctacatttaatactccatacatgtattcaaacattcgatatgacggcacgaaaatttttgttttgggaactaaatagggtcttagaggaaaagtaaagaatttggatttctcaggattaatttctatatgagttattcgatttgttaaaataaagtatgagaaagccaaaacaatattttctttcctacaagttgcatagagaaaaaaaaatccactcaaacctctaattttttttctatagattgatatgtacatatatttctatcccttcacttttcctattcttttccgcatgaagaaaaaaaagagaacttttctaaccatctctgcacttccg

SEQ ID NO: 11 - DNA, 1300 bp, Oryza sativa
tggacagcaggctacagatttatagccagctgtagcacggactttaagatgcatatgtatatgacagatgagaccagatattaattatgtagtatatgtttatatgtaactactgtatgaattgactattaaattgactatagatgatttggagtccgcagttagctttactactaaacttgctcttaatggaatgttccatcccaaagttcatcattctgtttggttagcattagagcaggtacaatagcagactattaaccagctataaacatattttaatgagataaaaagataagaaaaaagagcagcgggctataaatctgcagccggttgtagcacggactctaagatgcaatgtgtgtataatagataggactatatactaatagtatagtaaacaattattgtataaattagctattagattagctatagatgaattagagttagtagtgggctatactattaaacttgctcttagcatgcggttagagtgacagcaacatcggttcatcgatcaagagtagattatgagtggattataagttgtaatagcttactagtgaaaaatattttacgagtaaaacttttatatatacacatccataaatttttataatttttaaaataaatgcattaatgatacctaaaatccacttaaaattaagttttaaaatttaaactttgacgtcgattaatttgttatacgtcaatcaataagaccctttagctattgcacctaccgtatttttcctggatataaagtaagatcctgggatcaaagaaagcttcgcaatcacatgttgagaaaaggagtgggctttcagaagcaggagcagaaaatgatgtatgatagactagttcggtactgcagttaggtgcccataaagaagatcttccggtgattgcattgggaatgttcggttgcatttggagctagtatttggctatagtattatactagtattgaacttgctttaacagatttcttcagcttaataatttactgtttgaatttgttctacattttgagcagctttgatggtttctttgtgtagttttttcaagtataaaccaactcgatcaaagttaggaacagtacagatagaaggcaacaatgtcatatcagaggagagaatcattcctgtgttacgtggagtataaacaatctgcaatactctggtcattgctacatctgacaaaagtcgttgtgttaccacttaccaccatgtcgttcaagcagagctggagaatcttttcctcaattttctgttcggttttcagttccaccttgcct

(19) United States
(12) Patent Application Publication
(10) Pub. No.: US 2009/0229014 A1
(43) Pub. Date: Sep. 10, 2009

(54) Drought and High Salinity Stress Induced Rice Promoters

(76) Inventor: Xing Wang Deng, Hamden, CT (US)

Correspondence Address: BACHMAN & LAPOINTE, P.C., 900 CHAPEL STREET, SUITE 1201, NEW HAVEN, CT, 06510, US

(21) Appl. No.: 11/733,102

(22) Filed: Apr. 9, 2007

Related U.S. Application Data
(60) Provisional application No. 60/744,433, filed on Apr. 7, 2006.

(51) Int. Cl. A01H 5/00; C07H 21/04

(52) U.S. Cl. 800/288; 800/320.2; 536/23.6

(57) ABSTRACT

Rice promoters described herein promote the expression of drought and high-salinity stress responsive genes of rice so as to enable young and adult rice plants to withstand drought and high salinity stresses.

### CROSS-REFERENCE TO RELATED APPLICATION

This non-provisional patent application claims priority to U.S. Provisional Application No. 60/744,433 entitled “Genome Wide Identification of Rice Promoters with Distinct Organ-Specificity in response to Drought and High-Salinity Stresses”, filed on Apr. 7, 2006 in the name of Xing Wang Deng, which is incorporated by reference herein in its entirety.

### FIELD OF THE INVENTION

The invention relates to rice promoters and, more particularly, relates to drought and high salinity stress induced rice promoters.

### BACKGROUND OF THE INVENTION

Drought and highly saline soils are among the most serious challenges to crop production in the world today. This is particularly the case in developing countries, where these abiotic stresses severely limit crop growth and productivity. Both traditional breeding and genetic engineering of crop plants have been utilized to improve drought and high-salinity tolerance or resistance with the goal of increasing agricultural productivity in affected regions. Understanding plant responses to abiotic stresses at the genomic level provides an essential foundation for future breeding and genetic engineering efforts.

Recent research on drought and high-salinity responses in Arabidopsis implied that a large proportion of the genome is involved in drought (Shinozaki et al., “Molecular Responses to Dehydration and Low Temperature: Differences and Cross-talk Between Two Stress Signaling Pathways”, Curr. Opin. Plant Biol. 3:217-223 (2000); Shinozaki et al., “Regulatory Network of Gene Expression in the Drought and Cold Stress Responses”, Curr. Opin. Plant Biol. 6:410-417 (2003)), or high-salinity stress responses (Xiong et al., “Cell Signaling during Cold, Drought, and Salt Stress”, Plant Cell 141:S165-S183 (2002); Zhu, “Cell Signaling under Salt, Water and Cold Stresses”, Curr. Opin. Plant Biol. 4:401-406 (2001); Zhu, “Salt and Drought Stress Signal Transduction in Plants”, Annu. Rev. Plant Biol. 53:247-273 (2002); all said articles being incorporated herein by reference in their entireties). In several cases, it has been shown that alteration of individual gene expression level can significantly impact responses to drought (Garg et al., “Trehalose Accumulation in Rice Plants Confers High Tolerance Levels to Different Abiotic Stresses”, Proc. Natl. Acad. Sci. USA 99:15898-15903 (2002); Haake et al., “Transcription Factor CBF4 is a Regulator of Drought Adaptation in Arabidopsis”, Plant Physiol. 130:639-648 (2002)), or high-salinity stresses in plants (Kasuga et al., “Improving Plant Drought, Salt, and Freezing Tolerance by Gene Transfer of a Single Stress-Inducible Transcription Factor”, Nat. Biotechnol. 17:287-291 (1999); Seki et al., “Molecular Responses to Drought, Salinity and Frost: Common and Different Paths for Plant Protection”, Curr. Opin. Biotechnol. 14:194-199 (2003); Xu et al., “Expression of a Late Embryogenesis Abundant Protein Gene, HVA1, from Barley Confers Tolerance to Water Deficit and Salt Stress in Transgenic Rice”, Plant Physiol. 110:249-257 (1996); Zhang et al., “From Laboratory to Field. Using Information from Arabidopsis to Engineer Salt, Cold, and Drought Tolerance in Crops”, Plant Physiol. 135:615-621 (2004); all said articles being incorporated by reference herein in their entireties). Genome-wide identification of genes regulated by drought or high-salinity conditions has manifold significance. First, it provides a more comprehensive understanding of the transcriptional responses to those stresses. Second, it provides a starting point for further elucidating the role of individual genes in stress responses, which will be of great value in crop engineering. Third, it aids in the identification of stress responsive promoters and responsible cis-elements within them that are important both for basic study and crop engineering applications.

DNA microarrays provide a high throughput means of analyzing genome expression, which has been used to study patterns of gene expression in response to drought or high-salinity stresses in several plant species (Seki et al., “Molecular Responses to Drought, Salinity and Frost: Common and Different Paths for Plant Protection”, Curr. Opin. Biotechnol. 14:194-199 (2003); Seki at al., “RIKEN Arabidopsis Full-Length (RAFL) cDNA and its Applications for Expression Profiling under Abiotic Stress Conditions”, J. Exp. Bot. 55:213-223 (2004); all said articles being incorporated by reference herein in their entireties). Initially, a microarray containing ˜1300 full-length cDNA clones from Arabidopsis was used to study gene expression under drought and cold stresses. This study resulted in the identification of 44 and 19 cDNA clones as drought and cold-inducible genes, respectively (Seki et al., “Monitoring the Expression Pattern of 1300 Arabidopsis Genes under Drought and Cold Stresses by Using a Full-Length cDNA Microarray”, Plant Cell 13:61-72 (2001), and incorporated herein by reference in its entirety). Other studies employed an improved microarray containing around 7,000 Arabidopsis full-length cDNA clones to profile gene expression in response to abscisic acid (ABA) treatment (Seki et al., “Monitoring the Expression Pattern of Around 7,000 Arabidopsis Genes under ABA Treatments Using a Full-Length cDNA Microarray”, Funct. Integr. Genomics 2:282-291 (2002a), and incorporated by reference herein in its entirety), as well as cold, drought, and high-salinity stresses (Seki et al., “Monitoring the Expression of Profiles of 7,000 Arabidopsis Genes under Drought, Cold and High-Salinity Stresses Using a Full-Length cDNA Microarray”, Plant J. 31:279-292 (2002b), and incorporated herein by reference in its entirety). Another study employed an Affymetrix GeneChip covering approximately 8,100 genes from Arabidopsis to monitor changes in gene expression under salt, osmotic, and cold stresses. This study revealed that resulting expression changes varied significantly between root and leaf, with only minor overlap (Kreps et al., “Transcriptome changes for Arabidopsis in Response to Salt, Osmotic, and Cold Stresses”, Plant Physiol. 130:2129-2141 (2002), and incorporated by reference herein in its entirety). Similar studies have also been performed in barley to assess the drought and high-salinity gene expression responses using a microarray containing 1,463 DNA elements (Ozturk et al., “Monitoring Large-Scale Changes in Transcript Abundance in Drought- and Salt-Stressed Barley”, Plant Mol. Biol. 48:551-573 (2002), and incorporated by reference herein in its entirety).

Rice (Oryza sativa) is a model plant for cereal crops and has perhaps the richest set of resources available for plant genomic studies (Feng at al., “Sequence and Analysis of Rice Chromosome 4”, Nature 420:316-320 (2002); Goff et al., “A Draft Sequence of the Rice Genome (Oryza sativa L. ssp. japonica)”, Science 296:92-100 (2002); The Rice Chromosome 10 Sequencing Consortium (RCSC), “In-Depth View of Structure, Activity, and Evolution of Rice Chromosome 10”, Science 300: 1566-1569 (2003); Sasaki et al., “The Genome Sequence and Structure of Rice Chromosome 1”, Nature 420:312-316 (2002); Yu et al., “A Draft Sequence of the Rice Genome (Oryza sativa L. ssp. indica)”, Science 296:79-92 (2002); all said articles being incorporated by reference herein in their entireties). In rice, the high-salinity stress response has been analyzed with a microarray containing 1,728 cDNA clones from a root cDNA library of salt-tolerant rice (var Pokkali) (Kawasaki et al., “Gene Expression Profiles during the Initial Phase of Salt Stress in Rice”, Plant Cell 13:889-905 (2001), and incorporated by reference herein in its entirety). Another study with a microarray containing 8,987 DNA elements detected 509 possible abscisic acid (ABA)—or gibberellin-responsive genes (Yazaki et al., “Genomics Approach to Abscisic Acid- and Gibberelin-Responsive Genes in Rice”, DNA Res. 10:249-261 (2003), and incorporated by reference herein in its entirety). A total of 73 genes induced by cold, drought, high-salinity and ABA treatment were further identified using a microarray containing 1,700 full-length cDNA clones (Rabbani et al., “Monitoring Expression Profiles of Rice Genes under Cold, Drought, and High-Salinity Stress and Abscisic Acid Application Using a cDNA Microarray and RNA Gel-Blot Analyses”, Plant Physiol. 133:1755-1767 (2003), and incorporated by reference herein in its entirety). Recently, an Affymetrix rice genome array containing 55,515 probe sets was used to profile the transcriptomes of rice strains with salt-tolerant and salt-sensitive genotypes, revealing genome-wide differential transcription under salinity treatments during vegetative growth (Walia et al., “Comparative Transcriptional Profiling of Two Contrasting Rice Genotypes under Salinity Stress during the Vegetative Growth Stage”, Plant Physiol. 139:822-835 (2005), and incorporated by reference herein in its entirety). Taken together, previous studies in rice have identified various genes regulated by different stress conditions. However, a systematic comparison of whole-genome expression responses to drought and high-salinity stresses in various organs has not yet been performed.

The availability of the complete genome sequences of two rice sub-species (Sasaki et al., “The Genome Sequence and Structure of Rice Chromosome 1”, Nature 420:312-316 (2002); Yu et al., “The Genomes of Oryza sativa: A History of Duplications”, PLoS Biol. 3:e38 (2005); both said articles being incorporated by reference herein in their entireties) makes the construction of a whole-genome microarray possible. A whole-genome 70 mer oligomer microarray for rice was developed and successfully employed to obtain genome expression profiles (Ma et al., “A Microarray Analysis of Rice Transcriptome and its Comparison to Arabidopsis”, Genome Res. 15:1274-1283 (2005b); Jiao et al., “Conservation and Divergence of Light-Regulated Genome Expression Patterns during Seedling Development in Rice and Arabidopsis”, Plant Cell 17: 3239-3256 (2005); both said articles being incorporated by reference herein in their entireties). Here, we use this whole-genome microarray to monitor expression changes for a total of 36,926 genes in response to drought and high-salinity stresses in shoots at the four-tiller stage and in flag leaves and panicles at one week prior to heading. This analysis revealed the extent of reprogramming and alteration of cellular pathways in response to drought and high salinity stresses. The possible underlying mechanism for the observed reprogramming of the genome expression was examined.

Therefore, there exists a need for a rice promoter that promotes the expression of drought and high-salinity stress responsive genes of rice so as to enable young and adult rice plants to withstand drought and high salinity stresses.

### SUMMARY OF THE INVENTION

In one aspect of the present disclosure, a drought induced rice promoter broadly comprises a sequence listing of SEQ ID NO: 1. In another aspect of the present disclosure, a drought induced rice promoter broadly comprises a sequence listing of SEQ ID NO: 2. In yet another aspect of the present disclosure, a drought induced rice promoter broadly comprises a sequence listing of SEQ ID NO: 3. In still yet another aspect of the present disclosure, a drought induced rice promoter broadly comprises a sequence listing of SEQ ID NO: 4. In another aspect of the present disclosure, a drought induced rice promoter broadly comprises a sequence listing of SEQ ID NO: 5. In yet another aspect of the present disclosure, a drought induced rice promoter broadly comprises a sequence listing of SEQ ID NO: 6. In still yet another aspect of the present disclosure, a drought induced rice promoter broadly comprises a sequence listing of SEQ ID NO: 7. In another aspect of the present disclosure, a drought induced rice promoter broadly comprises a sequence listing of SEQ ID NO: 8. In yet another aspect of the present disclosure, a drought induced rice promoter broadly comprises a sequence listing of SEQ ID NO: 9. In still yet another aspect of the present disclosure, a drought induced rice promoter broadly comprises a sequence listing of SEQ ID NO: 10. In still yet even another aspect of the present disclosure, a drought induced rice promoter broadly comprises a sequence listing of SEQ ID NO: 11.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1A is a representation of the number of differentially expressed genes induced or expressed at each stage of drought and high-salinity treatments in the flag leaf;

FIG. 1B is a representation of the number of differentially expressed genes induced or expressed at each stage of drought and high-salinity treatments in the panicle;

FIG. 1C is a representation of the number of differentially expressed genes induced or expressed at each stage of drought and high-salinity treatments in the shoot;

FIG. 2A is a representation of the total number of drought and high-salinity stress inducible genes and the number of genes induced in response to both stresses in each of the three rice organs;

FIG. 2B is a representation of the total number of drought and high-salinity stress repressed genes and the number of genes repressed in response to both stresses in each of the three rice organs;

FIG. 2C is a representation of a clustering analysis of all drought and high-salinity responsive genes in each of the three rice organs;

FIG. 2D is a representation of the relatedness of the genome expression patterns across selected stress-treated rice organs;

FIG. 3A is a representation of a cluster analysis of the genes exhibiting significant elevation of expression after 48 hours of rehydration treatment for the flag leaf;

FIG. 3B is a representation of a cluster analysis of the genes exhibiting significant elevation of expression after 48 hours of rehydration treatment for the shoot;

FIG. 3C is a representation of a cluster analysis of the genes exhibiting significant elevation of expression after 48 hours of rehydration treatment for the panicle;

FIG. 3D is a representation of a histogram showing the number of genes in each of the three rice organs induced under drought treatment at least at one drought stage, after 48-hour rehydration period following the D3 stage, and the number of genes with overlapping expression in various pairs of organs;

FIG. 4A is a representation of a Venn diagram of drought induced genes among the three rice organs;

FIG. 4B is a representation of a Venn diagram of high-salinity induced genes among the three rice organs;

FIG. 4C is a representation of a Venn diagram of drought repressed genes among the three rice organs;

FIG. 4D is a representation of a Venn diagram of high-salinity repressed genes among the three rice organs;

FIG. 5 is a RT-PCR analysis of the representative drought-induced genes among an untreated control plant (S0, F0, P0) and the shoot (S1, S2, S3), flag leaf (F1, F2, F3), and panicle (P1, P2, P3) of the rice plant;

FIG. 6A is a graphical representation of ABRE core motif distribution among promoters of the genes induced only be drought or only by high-salinity stress in each single rice organ;

FIG. 6B is a graphical representation of ABRE core motif distribution among promoters of genes induced by both drought and high-salinity stress in each rice organ;

FIG. 6C is a graphical representation of ABRE core motif distribution among promoters of genes induced in two or three organs;

FIG. 6D is a graphical representation of DRE core motif distribution among promoters of genes induced only by drought or only be high-salinity stress in each rice organ;

FIG. 6E is a graphical representation of DRE core motif distribution among promoters of genes induced by both drought and high-salinity stress in each rice organ;

FIG. 6F is a graphical representation of DRE core motif distribution among promoters of genes induced in two or three organs or by both stresses in more than one organ;

FIG. 7A is a representation of the core sequence of the motif and the position of the motif in each promoter;

FIG. 7B is a representation of an expression pattern of a representative gene containing motif-SP;

FIG. 7C is a representation of a gel shift assay of motif-SP;

FIG. 8 is a representation of three motifs associated with genes repressed during drought and induced by rehydration in panicle;

FIGS. 9A-D are photographs of the sampling stages of the rice plants during drought stages D1, D2 and D3 and various stages of rolling exhibited by the flag leaves;

FIG. 10 is a representation of a gene ontology analysis of drought and high-salinity stressed responsive genes in the flag leaf, panicle and shoot;

FIGS. 11A-11G are representations of the effects of drought and high-salinity on rice genes coding enzymes in known metabolic pathways using the shoot as a model;

FIG. 12 is a photograph illustrating the selection of T1 transgenic lines with Hygromycin with the left side containing wild rice Zhongua 11 and the right side containing T1 transgenic rice;

FIG. 13 are photographs indicating GUS activity in transgenic rice KT220 and KT261 was drought inducible in all tissue types;

FIG. 14 are photographs indicating GUS activity in transgenic rice KT247 and KT270 was drought inducible in both shoot and flag leaves;

FIG. 15 are photographs indicating GUS activity in transgenic rice KT250 and KT274 was drought inducible only in panicles; and

FIG. 16 represents the GUS activity of five exemplary promoters displaying drought-inducible GUS-expression in seedlings and flag leaves.

Like reference numbers and designations in the various drawings indicate like elements.

### DETAILED DESCRIPTION

For use herein, differentially expressed genes refers to both induced and repressed genes having a P values <0.05 and exhibiting a 3-fold or more difference in expression change.

The inventors of the present disclosure examined whole genome expression profiles under drought and high-salinity conditions in three rice plant organs: four-tiller stage shoot, filling stage flag leaf and panicle. Rice plants at these two growth stages, particularly the late one, are sensitive to drought and high-salinity stress. It is well known to one of ordinary skill in the art that drought or high-salinity stress at the heading and early panicle stages of rice plants can severely compromise rice growth and development and reduce crop yield even with late rehydration. Evidence strongly suggests that the rice genome is subject to significant reprogramming with regard to which portion of genome is expressed under drought or high salinity stress.

The inventors of the present disclosure monitored the expression of 36,926 unique or known rice genes or gene models in the aforementioned rice plant organs under drought and high salinity stress. The present disclosure offers the first comprehensive picture of genome expression modulation in the shoot, leaf and panicle of rice in response to drought and high salinity stress.

Using a whole genome microarray, a total of 2,957 and 2,090 rice genes showed significant up-regulation or down-regulation, respectively, in response to high salinity stress and drought stress in at least one of the three aforementioned rice organs. The analysis suggests that 927 out of 2,090 (44%) genes induced by drought were also up regulated by high-salinity stress in the same organs. In rice, an even higher percentage of drought induced genes were also up-regulated under high-salinity stress.

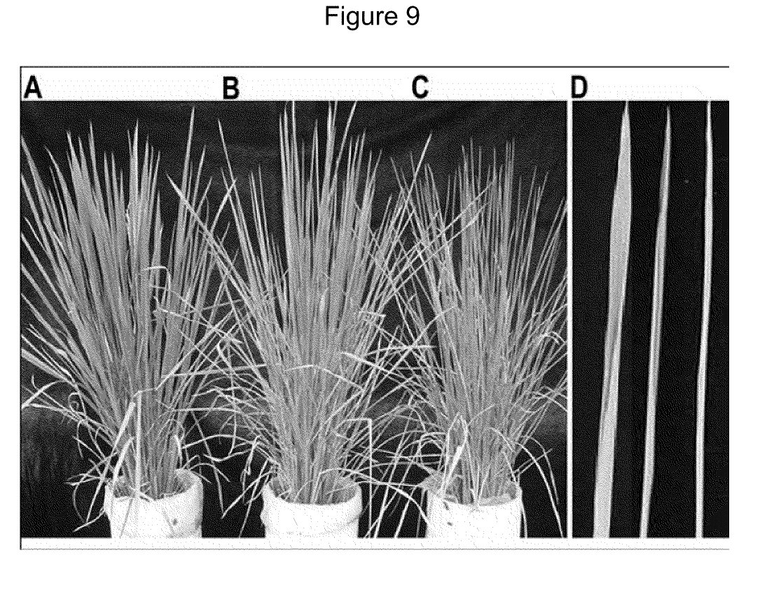

The inventors of the present disclosure note the overlap of up to about half of the genes induced or inhibited by drought and high-salinity stress in rice. This observation is consistent with current understanding that these two stresses affect plants in overlapping but not identical ways. At a physiological level, both stresses cause water depletion in the above-ground portion of the plants and induce similar morphological responses (See FIG. 9). Half of the transcriptional factor genes identified herein was shared by both stresses in each organ at the molecular level, which is consistent with the observation that about half of the genes that respond to the two stresses were shared at the whole genome level.

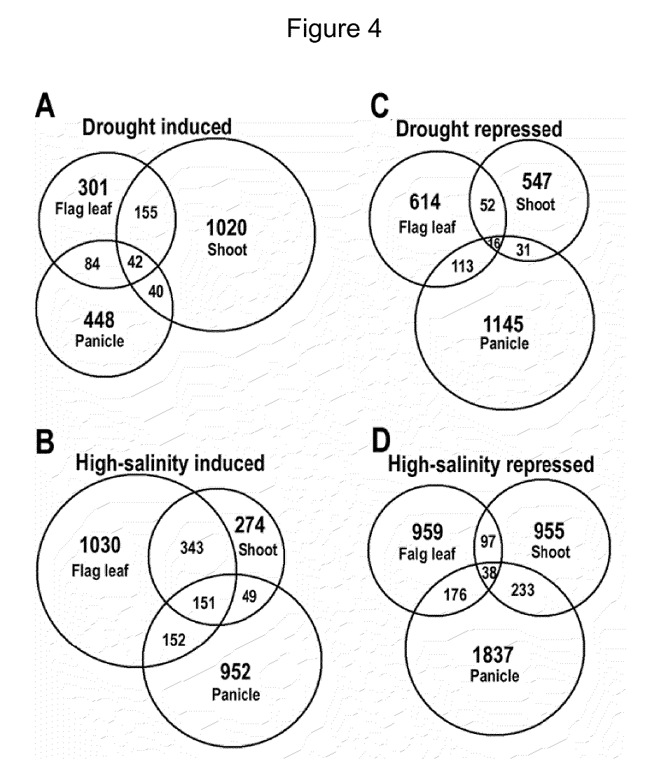

The results disclosed herein demonstrate that only a limited number of drought and high salinity responsive genes were shared between any two aforementioned rice plant organs. For instance, only 13.5% and 20.5% of drought-induced genes in the panicle were shared with those induced in the shoot and flag leaf respectively, and 33.8% of drought-induced genes in the shoot were also activated in the flag leaf (See FIGS. 4A-4D). The percentages of genes shared between any two aforementioned rice plant organs under high-salinity conditions were similar to those under drought stress, suggesting and confirming prior observations that responses to drought or high-salinity stress in different organs were independently regulated.

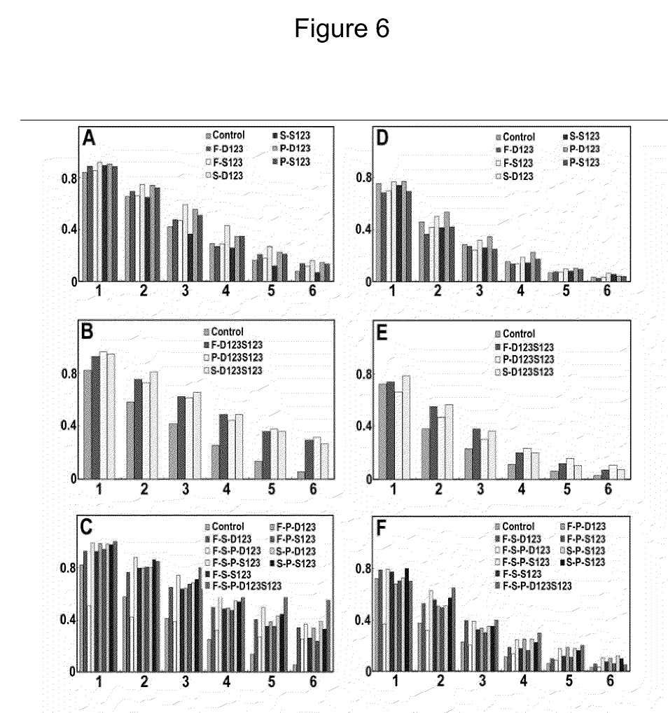

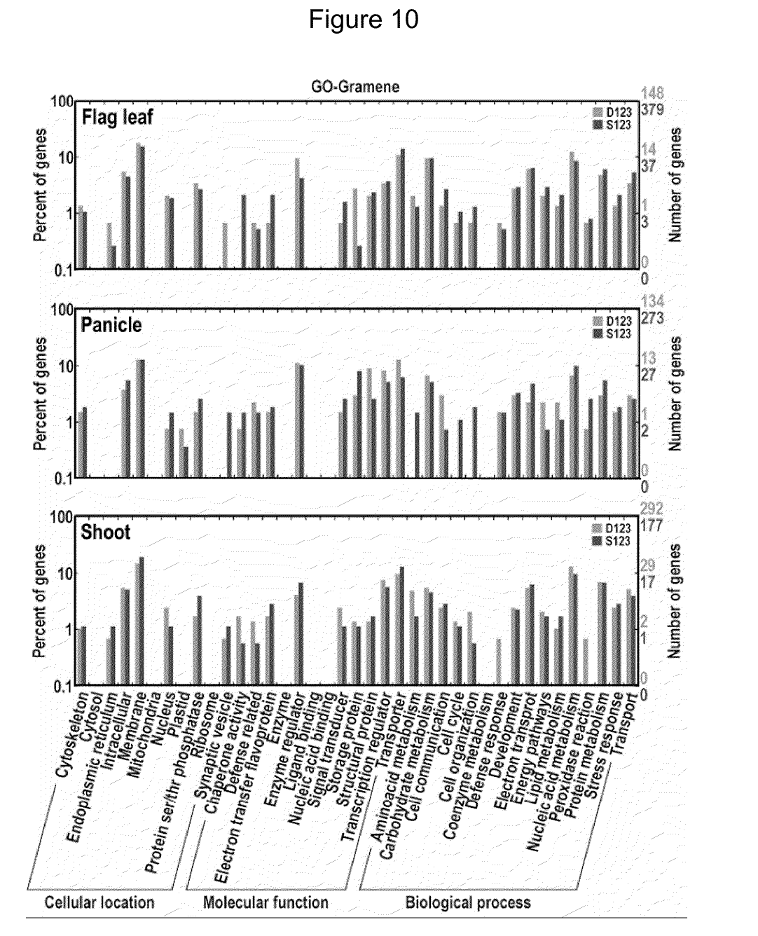

Genome expression reprogramming under either drought or high salinity stress entails a large number of genes involved in many aspects of cellular function. A gene ontology (“GO”) analysis of stress responsive genes (See FIG. 10) indicates that each organ activated similar categories of genes in response to both stresses, but the genes in each category are different among the three organs. It is thus possible that homologous or functionally similar genes in each category may show organ-specific expression in response to stresses. This is consistent with our observation of organ-specific transcription factor gene expression in response to both drought and high salinity stresses (See Table 3). The fact that genes specifically induced in each organ do not exhibit enrichment of DRE and ABRE motifs (See FIG. 6A-F) suggests that organ-specific transcriptional factor gene expression may be responsible for activating organ-specific downstream genes in a secondary transcriptional response to stress. For these genes, it is likely that certain organ-specific promoter elements mediate this response pathway.

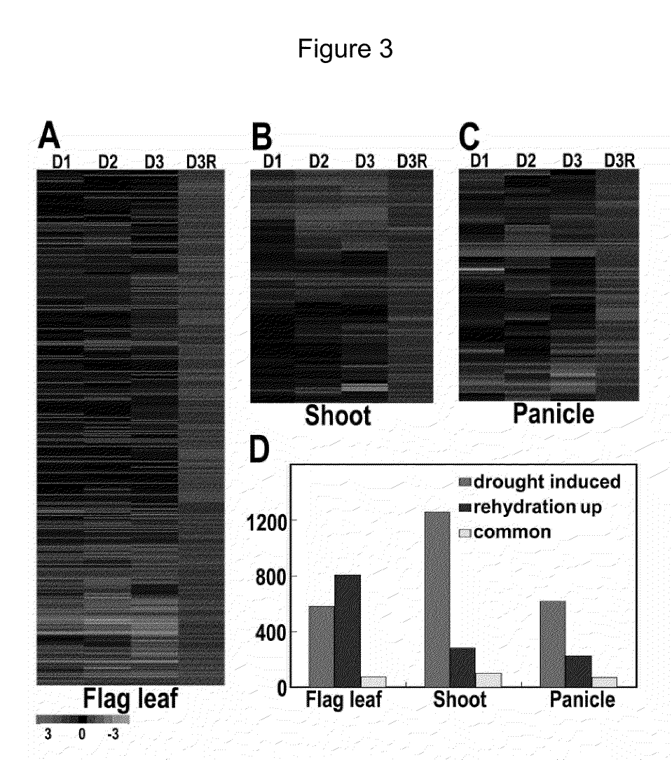

According to our microarray analysis, rice genes induced by 48-hour rehydration were divided into three groups according to their expression patterns (See FIGS. 3A-3D), as also done in a study of Arabidopsis reported in “Monitoring Expression Profiles of Arabidopsis Gene Expression During Rehydration Process After Dehydration Using ca. 7000 full length cDNA microarray” by Oono, Y. et al., Plant J. 34:868-887 (2003), incorporated by reference herein in its entirety. This observation suggests that monocotyledonous and dicotyledonous plants may share similar gene expression responses to rehydration after drought. Our analysis of the genome-wide distribution of rehydration-regulated genes in the aforementioned rice plant organs demonstrated no significant co-regulation of neighboring genes at the chromosomal level. Our observations based upon the analyses disclosed herein are similar to the case of light-regulated genes as reported in “Conservation and Divergence of Light-Regulated Genome Expression Patterns During Seedling Development in Rice and Arabidopsis” by Jiao Y. et al., Plant Cell. 17:3239-3256 (2005) incorporated by reference herein in its entirety, but different from general gene transcription from organ samples as reported in “A Microarray Analysis of the Rice Transcriptome and Its Comparison to Arabidopsis”, by Ma L. G. et al., Genome Res. 15:1274-1283 (2005b) incorporated by reference herein in its entirety. The inventors of the present disclosure note this distinction in the modulation of genome expression may reflect different mechanisms employed in response to different developmental or environmental signals as will be described further.

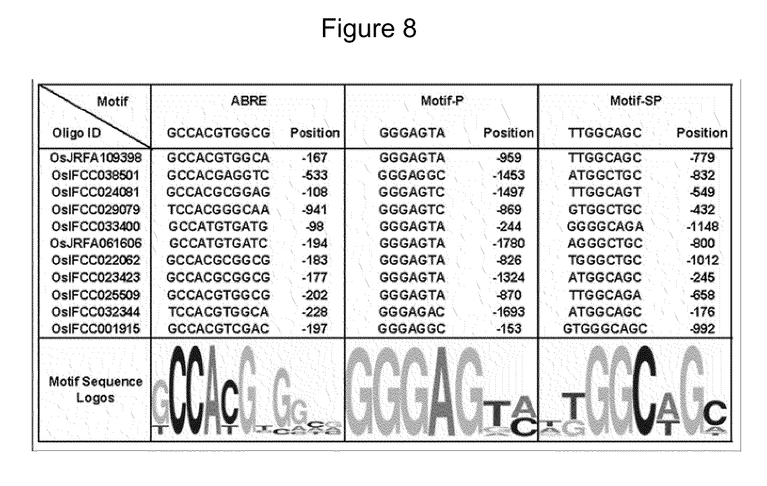

Up to now, only one possible cis-element has been reported to be involved in the rehydration process after dehydration in Arabidopsis as reported in “ACTCAT, A Novel Cis-Regulatory Element for Proline- and Hypoosmolarity-Responsive Expression of the ProDH Gene Encoding Proline Dehydrogenase in Arabidopsis”, by Satoh, R. et al., Plant Physiol. 130:709-719 (2002) incorporated herein by reference in its entirety, and in Oono, Y. et al. (2003). In rice, 807, 281 and 224 genes induced by rehydration in flag leaf, shoot and panicle separately, respectively, were identified at the whole genome level (See FIGS. 3A-3D). These genes served as an important starting point to study rehydration mechanisms and to search for novel cis-regulatory promoter elements associated with dehydration or rehydration. Two novel cis-regulatory elements, motif-SP and motif, were identified (See FIG. 8). Motif-SP was also found in shoot-specific genes with similar expression patterns in response to drought and rehydration. Gel shift assays provided evidence that motif-SP may function as a cis-element to mediate the drought-induced repression and late de-repression, i.e., activation, during rehydration in rice.

Several well-characterized drought-, high-salinity and cold inducible gene promoters may contain two common cis-regulatory elements, the ABA-responsive element (ABRE) and the dehydration-responsive element (DRE) as understood and recognized by one of ordinary skill in the art. ABRE and DRE confer ABA-dependent and ABA-independent gene expression in response to water stress. A single copy of ABRE in the promoter region only induces relatively minor elevation of expression level, while multiple copies of ABRE strongly activate expression as recognized by one of ordinary skill in the art.

Previous studies reported that drought stress suppressed plant photosynthesis systems and significantly modulated the activity of some membrane transporters according to “The Combined Effect of Drought Stress and Heat Shock on Gene Expression in Tobacco”, Rizhsky, L. et al., Plant Physiol. 130: 1143-1151 (2002); “Drought-Induced Responses of Photosynthesis and Antioxidant Metabolism in Higher Plants”, Ramachandra, R. A. et al., J. Plant Physiol. 161:1189-1202 (2004); “The Role of Aquaporins in Cellular and Whole Plant Water Balance”, Johansson, I. et al., Biochem. Biophys. Acta. 1465:324-342 (2004); and “Regulation of the ABA-Sensitive Arabidopsis Potassium Channel Gene GORK in Response to Water Stress”, Becker, D. et al., FEBS Lett. 554:119-126 (2003); all said articles being incorporated herein by reference in their entireties. The microarray analysis disclosed herein indicated genes involved in photosynthesis and genes encoding transporters were repressed or maintained at low levels of expression under drought but were then strongly activated after rehydration. The repression of metabolic genes during drought stress allows the plant to conserve energy and subsist on less water, thus conferring better drought tolerance. When supplied water upon rehydration, activation of these genes may aid in the recovery of full photosynthesis activity and transmembrane solute/water exchange, thus helping a rice plant resume its normal growth and development quickly.

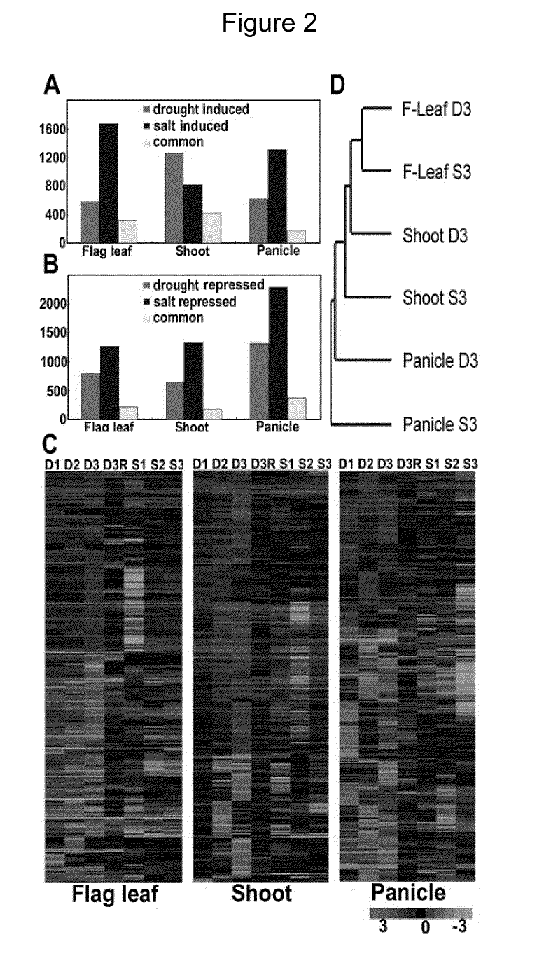

To compare the gene expression profiles of the drought and high-salinity stress responses, the inventors of the present disclosure examined genes commonly regulated by both stresses at each organ type. A total of 322, 415, and 174 genes were up-regulated and 215, 173, and 372 genes were down-regulated by both stresses in the aforementioned flag leaves, shoots and panicles, respectively (SEE FIGS. 2A and 2B). The common genes represent 55%, 33% and 28% (induced) and 27%, 27% and 29% (repressed) of all drought-responsive genes in the aforementioned flag leaf, shoot and panicle, respectively. Our results disclosed herein indicate that about one-third, and even half in flag leaf, of the drought responsive genes in each aforementioned organ are also regulated by high salinity stress.

To further compare gene expression under the two abiotic stresses, all drought regulated genes were selected for cluster analysis. The induced and repressed differentially expressed genes having a p value <0.05 and exhibiting a 3-fold or more in expression change at least one stage of drought treatment were included. The median ratio of treated to untreated sample was log 2 transformed and subject to complete linkage hierarchical clustering. D3R refers to the 48-hour water recovery after drought. In total, 1,377, 1,903 and 1,919 genes from flag leaf, shoot and panicle, respectively, were included. As shown in FIG. 2C, up to half of the genes up-regulated or down-regulated by drought stress also exhibited a similar expression pattern under high-salinity stress. The remainder of the drought responsive genes exhibited minimal expression changes or distinct expression patterns in response to high-salinity stress.

Among genes specifically induced by drought stress, some have known functions while others have been previously predicted to have functions related to drought stress or the ABA response. For example, our results disclosed herein demonstrate that genes with putative functions including NAM, HLH, G-box binding, Zn-finger, AP2 transcription factors, and protein kinases (including MAPK family genes), including a few known drought-responsive genes such as DREB1A, LEA protein, WS176 protein, MAP65 (microtubule associated protein), and ubiquitin, were all affected by drought stress. The few known drought-responsive genes were specifically induced by drought and may function exclusively in the response to drought stress in rice. To gain an overall view of the effects of drought and high-salinity stress on various functional gene groups, GO categories for drought and high-salinity stress induced genes in the aforementioned three organs were examined (See FIG. 10). For the majority of these functional categories, our analysis indicated the relative numbers of genes responsive to the two stresses were similar.

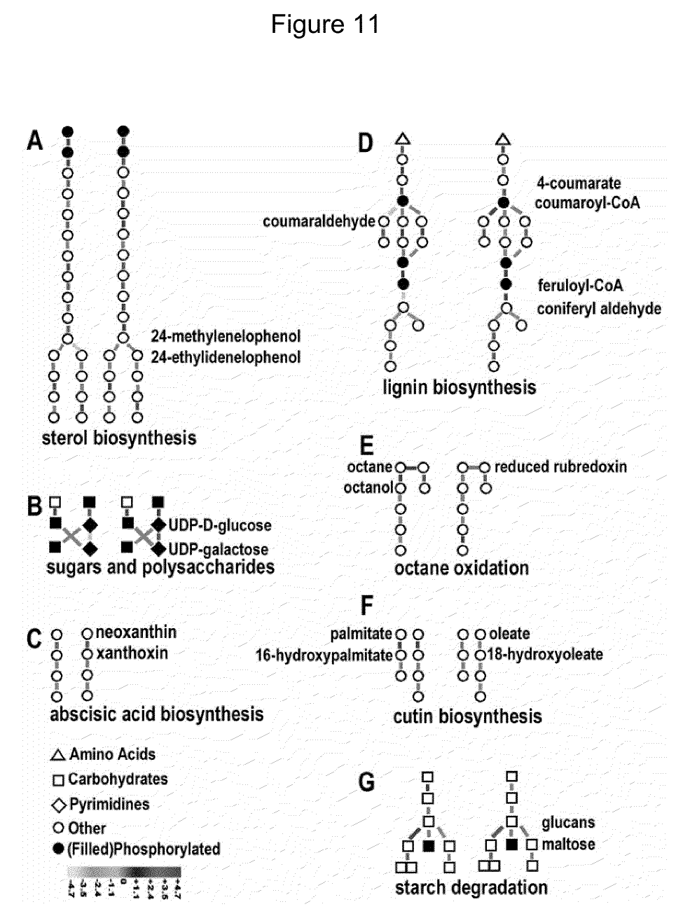

The effects of drought and high-salinity on rice genes coding enzymes in known metabolic pathways were also examined using the shoot as a model (See FIG. 11). Some pathways are similarly regulated by the two stresses, including the Calvin cycle, TCA cycle variation I, brassinosteroid biosynthesis II, gibberellin biosynthesis, and IAA biosynthesis I, and the de novo biosynthesis of pyrimidine ribnucleotides/pyrimidine deoxyribonucleotides/purine nucleotides (See FIGS. 11A-11G). While other pathways respond differentially to drought or high-salinity stresses, the representative pathways include sterol biosynthesis, sugars and polysaccharides, abscisic acid biosynthesis, lignin biosynthesis, octane oxidation, cutin biosynthesis, and starch degradation (See FIGS. 11A-11G). For example, some steps in these pathways were repressed or invariable under high-salinity stress, but were induced by drought stress. It is noteworthy that one step in the ABA-biosynthesis pathway showed no change in gene expression under high-salinity stress, but was induced under drought treatment (See FIG. 11C), suggesting that ABA is an osmosis-responsive factor and may play a role in the response to drought stress but not high-salinity stress.

By comparing transcriptomes under drought and high-salinity stresses in the three aforementioned rice organs, the inventors of the present disclosure discovered that drought responsive genome expression in flag leaves is closely related to that for high salinity stress than to the drought expression response observed in any other aforementioned rice organ as illustrated in the complete-linkage hierarchical clustering analysis of overall relatedness for expression ratios from selected organs at the third stage of both abiotic stress treatments of FIGS. 2C and 2D. In general, there seems to be a closer relationship between the transcriptome-level responses to the two abiotic stresses in the same organ than between or among transcriptomes in distinct organs in response to the same stress. However, the extent of overlap in the responses to drought or high-salinity stresses varies for the different rice organs. For example, the responses to the two abiotic stresses in panicle are more divergent, while responses to the two abiotic stresses in flag leaves show the greatest overlap.

In its natural environment, a plant's ability to respond to rehydration after drought stress is important for its survival, although little is known about the gene expression changes that occur during rehydration. Our analysis and results disclosed herein demonstrated that after a period of drought (third stage), all the rice leaves rolled and turned yellow, and the relative water content of the stressed plants dropped to 70-75%. When we applied water to those severely stressed plants, their rice leaves started to unroll after 5 hours, and exhibited normal flatness within 24-48 hours of rehydration. Tissue samples were collected 48 hours after rehydration and total RNA samples were extracted for gene expression analysis (FIG. 2C).

When compared to unstressed rice plants, in flag leaf, shoot and panicle, 807, 281 and 224 genes were up-regulated after 48 hours of rehydration following drought stress, respectively (See FIGS. 3A-3D; Supplemental Table 13, 14 and 15). Gene expression profiles were compared further during drought treatment with those after rehydration (See FIGS. 3A-3C). The expression patterns of those genes seem to belong to three groups, with flag leaves exhibiting the greatest number of gene expression changes following rehydration. Group I, which included 71, 98 and 68 genes from the flag leaf, shoot and panicle, respectively, was induced during both drought treatment and rehydration. Group II, which included 71, 8 and 21 genes in the flag leaf, shoot and panicle, respectively, was repressed during drought stress but induced by rehydration for shoot and panicle (See Table 1). Group III, which included all remaining 665 genes in the flag leaf, 175 genes in the shoot, and 135 genes in the panicle, showed no significant expression changes under drought stress but were up-regulated during rehydration. The analysis and results disclosed herein suggest that group I genes are induced by drought stress, but did not return to normal levels after 48-hour rehydration period. In contrast, those genes in groups II and III were specifically induced by rehydration.

The down-regulation of group II genes in response to drought and rehydration suggest these genes may play an important role in conferring drought stress tolerance, whereas the up-regulation of group II and group III genes during rehydration may be important for recovery. In both group II and III genes, two classes of genes, transporter genes and photosynthesis-related genes, were over-represented among the rehydration-inducible genes in shoot and panicle. These transporter genes included the following genes: OsJRFA065471 (folate/biopterin transporter), OsJRFA066919 (putative potassium transporter), OsJRFA067899 and OsIFCCO 19970 (ABC transporter, putative), OsJRFAO68003 and OsJRFA 106202 (Transmembrane amino acid transporter protein), OsJRFA068765 (H-ATPase), OsIFCC040482 (symporter), Os1RFA072183 (Sodium:sulfate symporter transmembrane region), OsJIRF′A102086 (putative lipid transfer protein) and OsJRFA 103807 (aquaporin). The photosynthesis related genes cover most of the gene components of the two photosystems, such as genes for putative chlorophyll a/b-binding protein, Photosystem I or II reaction center subunits, and plastocyanin. These photosystem genes and transporter genes represent a large portion of all the genes induced by rehydration, and their physiological roles in plants fit well with a potential contribution toward plant recovery from drought stress.

The degree of overlap in expression of stress responsive genes in two or more rice organs under drought or high-salinity stress was also examined. Referring specifically to FIGS. 4A-4D, Venn diagrams revealed that only a small portion of genes was shared between each pair of organs. The greatest overlap occurred between shoot and flag leaf, which shared more inducible genes than shoot and panicle or flag leaf and panicle under both drought and high-salinity conditions. Under drought stress, one-third of the genes up-regulated in the flag leaf (197/582) were also induced in the shoot (FIG. 4A), while under high salinity stress, over half of the induced genes (494/817) in the shoot (mostly young leaves) were also up-regulated in the flag leaf (FIG. 4B). Only about 21% and about 23% of genes up-regulated in panicle were also induced in flag leaf under drought and high salinity stresses, respectively. The percentage of genes shared between the shoot and panicle is similar to the percentage shared between flag leaf and panicle (See FIGS. 4A and 4B).

Our analysis and results disclosed herein demonstrated only a small fraction of stress responsive genes were expressed in all three organs. For example, 42 and 151 genes were induced in all three aforementioned organs under drought and high-salinity stress, respectively. In general, most of those genes exhibited high levels of expression as well as strong inducibility. Among them, 27 genes were induced by both drought and high-salinity stress in all three organs. This group of 27 genes includes a protein kinase (OsJRFA058518), chlorophyll a/b binding protein (OsJRFAO62972), ABA-responsive protein (OsJRFA063578, OsIFCCO18156), LEA protein (OsJ1RFA063984), dehydrin (OsIFCCO35028), CHY zinc finger protein (OsIFCCO03263), and homeobox protein (OsIFCCO18343) (See Table 2).

Analysis of stress down-regulated genes (See FIGS. 4C and 4D) revealed a similar small overlap among the three aforementioned organs. Only 68 and 129 out of 795 total genes down-regulated in flag leaf under drought stress were also inhibited in the shoot and panicle, respectively. For high-salinity stress, 135 and 214 out of 1,270 down-regulated genes in the flag leaf were also repressed in the shoot and panicle, respectively. Only 16 and 38 genes were down-regulated in all three organs under drought and high salinity stress respectively, while only two genes were inhibited in all three organs under both drought and high salinity stress (See Table 2).

Transcription factor genes whose expressions are regulated by drought and high-salinity stresses were also examined further. Among all genes induced by drought or high-salinity treatment in the three organs, a total of 186 genes (Supplemental Table 16) were predicted to be transcription factors with a DNA-binding domain. These transcription factors belong to various families, including AP2, bHLH, MYB, HB, NAC, zinc finger, MADS, bZIP, WRKY and HSF families as described in (Gong et al., 2004) incorporated by reference herein in its entirety. These transcription factors could also be divided into several groups depending on their expression patterns. Among the 186 transcription factor genes, 12 genes were induced in all three aforementioned organs in at least one stage following either drought or high-salinity stress, whereas the remaining 174 genes were mainly up regulated in one or two organs. Transcription factors induced only in one individual organ were listed in Table 3. It is interesting to note that over half of the transcription factors were expressed in at least two organs, while other transcription factors were activated in an organ-specific manner. This suggests that the expression of different transcription factors may play a key role in common or organ-specific gene expression in response to drought or high-salinity conditions.

To elucidate the relationship between copy numbers of DRE and ABRE cis-regulatory elements in gene promoter regions and abiotic stress-induced gene transcription, the inventors of the present disclosure divided the genes into groups based on their expression patterns. Genes induced specifically by drought or high-salinity stress in each of the three aforementioned organs defined six groups, while genes induced by both drought and high-salinity stress in each organ defined another three groups. Common inducible genes shared by at least two aforementioned organs under drought or high-salinity stress defined another nine groups. Only those genes among all those groups with full-length cDNA sequences available for further promoter analysis were selected. In brief, the 2 Kb upstream regions (−1 to −2,000 bp) preceding the ATG start codon of genes were selected for promoter motif analysis (see Materials and Methods). A similar number of rice genes with full length cDNA available that lack stress responsive expression were used as control. The copy number of ABRE and DRE core motifs on each promoter region was counted. The percentages of genes in each group with 1 to 6 copies of ABRE core or DRE core elements were then calculated.

The following 18 gene groups (with number of genes with full-length cDNA gene number) were analyzed: (1) Genes induced by drought stress only in flag leaf (F-D123, 82); (2) Genes induced by high-salinity stress only in flag leaf (F-S123, 433); (3) Genes induced by drought stress only in shoot (S-D123, 272); (4) Genes induced by high-salinity stress only in shoot (S-S123, 77); (5) Genes induced by drought stress only in panicle (P-D123, 139); (6) Genes induced by high-salinity stress only in panicle (P-S123, 420); (7) Genes induced by both stresses in flag leaf (F-D123S123, 190); (8) Genes induced by both stresses in shoot (S-D123S123, 252); (9) Genes induced by both. stresses in panicle (P-D123S123, 77); (10) Genes induced by drought stress in both flag leaf and shoot (F-S-D 123, 123); (11) Genes induced by drought stress in all three organs (F-S-P-D123, 59); (12) Genes induced by high-salinity stress in all three organs (F-S-P-S123, 97); (13) Genes induced by high-salinity stress in both flag leaf and shoot (F-S-S123, 309). (14) Genes induced by drought stress in both flag leaf and panicle (F-P-D123, 59); (15) Genes induced by high-salinity stress in both flag leaf and panicle (F-P-S123, 184); (16) Genes induced by drought stress in both shoot and panicle (S-P-D123, 51); (17) Genes induced by high-salinity stress in both shoot and panicle (S-P-S123, 124); and (18) Genes induced by both stresses in all three organs (F-S-P-D 123 S123, 20).

Compared to control genes, copy numbers of the ABRE and DRE core motifs in the promoter regions of genes with organ-specific expression in response to drought or high-salinity stress were not markedly different (See FIGS. 6A and 6D). The promoter regions of genes responsive to both drought and high-salinity stress in each organ were enriched for ABRE and DRE core motifs compared to the promoters of control genes (See FIGS. 6B and 6E). For example, only 25% of control genes have over four copies of ABRE core motif in their promoter regions, but 48%, 44% and 48% of genes induced by both drought and high-salinity stresses in flag leaf, panicle and shoot respectively have over four copies of the ABRE core motif in their promoter regions (See FIG. 6B).

When genes expressed in at least two organs under drought or high-salinity stress were compared to control genes, the ABRE and DRE core motif copy numbers also showed significant differences (See FIGS. 6C and 6F). For example, 50% of these genes contain over four copies of the ABRE core motif in their promoter regions, compared with 25% for the control genes (See FIG. 6C). A similar pattern was found for the DRE core elements (See FIG. 6F).

Genes induced in an organ-specific manner by drought or high-salinity stresses did not contain a significantly different number of copies of ABRE or DRE core motifs compared to control genes (See FIGS. 6A and 6D). Thus it is plausible that the stress-induced expression in this fraction of genes may rely less on transcriptional activation mediated by ABRE and DRE motifs. Unidentified organ-specific cis-regulatory elements may exist in the promoter regions of these genes and play a more important role in transcription activation under drought or high-salinity stress.

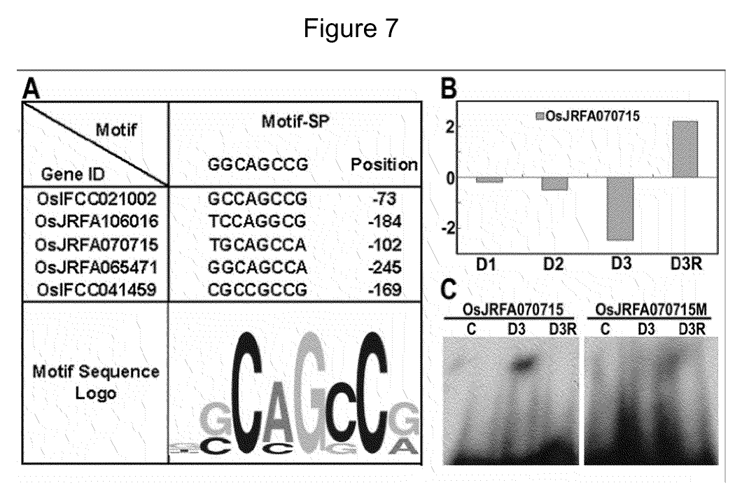

As shown in Table 1, there are 8 and 21 genes with 11 full-length cDNA sequences repressed by drought stress but induced by rehydration in shoot and panicle, respectively. Referring to FIGS. 7A-7C, representations of a novel promoter motif associated with genes repressed by drought but induced after rehydration in the rice shoot are shown. Nuclear proteins were extracted from shoots of untreated control plants (C), plants at stage 3 of drought treatment (D3), and plants after 48-hour rehydration following stage 3 (D3R). The core sequence of the probe is OsJRFAO7O715: TGCAGCCA, and the core sequence of the probe with a single base point mutation is OsJRFAO7O715M: TGAAGCCA. An analysis of the upstream region of these eight shoot genes identified a novel motif (motif-SP, GGCAGCCG) located near to the translation start codon (−73 to −250) in five of them (See FIG. 7A). To investigate the potential functional role of this novel motif, we performed a gel shift mobility assay which involved incubating a 24 bp probe containing motif-SP from one of those five genes (OsJRFAO7O715) and a control containing a single base mutation at an invariable position with nuclear extracts (see Materials and Methods). Only nuclear extracts from drought-treated shoot (at the 3rd stage), but not from unstressed shoot or shoot under rehydration, showed a sequence specific binding activity with the probe (FIG. 7C). A single base mutation at an invariable position in the motif-SP core of the probe completely abolished protein binding activity, suggesting high specificity of interaction with the core motif included in this 24 bp sequence. This specific binding activity was only observed in drought-treated plants, suggesting that it is involved in transcriptional repression under drought conditions.

The promoter regions of 21 genes inhibited by drought stress but induced by rehydration in panicle were also searched. Three conserved motifs were found through this blind search in 11 of the 21 genes: ABRE motif, the above-mentioned motif-SP, and another novel element, motif-P (FIG. 8). The presence of the ABRE motif suggests that a gene may respond to drought stress by employing an ABA-dependent pathway, while motif-SP may play an important role in drought-stress repression and subsequent activation during rehydration. We failed to detect specific binding activity using similar nuclear extracts (data not shown) and thus the role of Motif-P remains to be defined.

### EXPERIMENTAL SECTION

### Functional Classification

GO terms used in rice gene functional annotations were downloaded from the BGI-RIS database at http://rise.genomics.org.cn. For biochemical pathway analysis, genes were classified by associated biochemical pathway(s) using the AraCyc database (http://www.arabidopsis.org/tools/aracyc) for Arabidopsis, which is based on MetaCyc pathway collections. A rice gene was considered to be associated with a biochemical pathway if the rice gene had an Arabidopsis homolog in that pathway. The method used to identify highly homologous genes between rice and Arabidopsis is outlined in “The Genomes of Oryza sativa: A History of Duplications”, by Yu, J. et al., PLoS Biol. 3:e38 (2005) incorporated herein by reference in its entirety.

Plant Material and Stress Treatments

Plant material used in this study was Oryza sativa L. ssp. indica (cv. Minghui 63). Shoot samples were selected at the 4-tiller stage (vegetative stage) and flag leaf and panicle samples were selected at one-week-before-heading (reproductive stage). The plants used for shoot samples were grown in hydropolic half-strength Hoagland solution up to the 4-tiller stage, and then plants allowed to reach the reproductive stages were grown in soil. The shoot samples consisted largely of young leaves and were thus physiologically closer to flag leaf than panicle.

Total RNAs were isolated from four-tiller stage shoots and from flag leaves and panicles (as described in “Materials and Methods”) at three different time points (or sampling stages) after initiation of drought or high-salinity stress treatments (See FIG. 9).

The sampling stages for both drought and high-salinity treatments were based on morphological phenotypes, e.g. leaf rolling state, and physiological states, e.g., relative water content (“RWC”). Stage one is defined as exhibiting slightly rolled leaves with 90-95% relative water content. Stage two is defined as exhibiting halfway rolled leaves with 80-85% relative water content. Stage three is defined as exhibiting completely rolled leaves and with 70-75% relative water content. For drought treatment, the three stages were designated as D1, D2, and D3, while the three stages for the high-salinity treatment were designated as S1, S2, and S3 (See FIG. 9).

For drought treatment samples, materials at the following stages were collected: Stage 1 (D1), when leaves were slightly rolled and leaf relative water content (RWC) was approximately 90-95%; Stage 2 (D2), when leaves were half-rolled and leaf RWC was approximately 80-85%; Stage 3 (D3), when leaves were completely rolled and leaf RWC was approximately 70-75%. Rehydration samples were prepared by first subjecting the rice plants to drought treatment until the leaves were completely rolled (D3), then supplying enough water so that after 48 hours the plants recovered to normal appearance. Samples were then collected. For each treatment stage (D1, D2, D3), three independent replicates were collected for each sample.

For sample collection under high-salinity treatment (200 mM NaCl per plant), rice plants were collected when plants reached S1, S2, or S3 as outlined above for drought treatment. For each stage, three independent replicates were collected. Upon collection, samples were frozen immediately in liquid nitrogen and stored in a freezer at approximately −80° C. The estimation of RWC in rice plants has been previously reported in “A Re-Examination of the Relative Turgidity Technique for Estimating Water Deficit in Leaves”, by Barr, H. D. et al., Aust. J. Biol. Sci. 15:413-428 (1962) and incorporated by reference herein in its entirety.

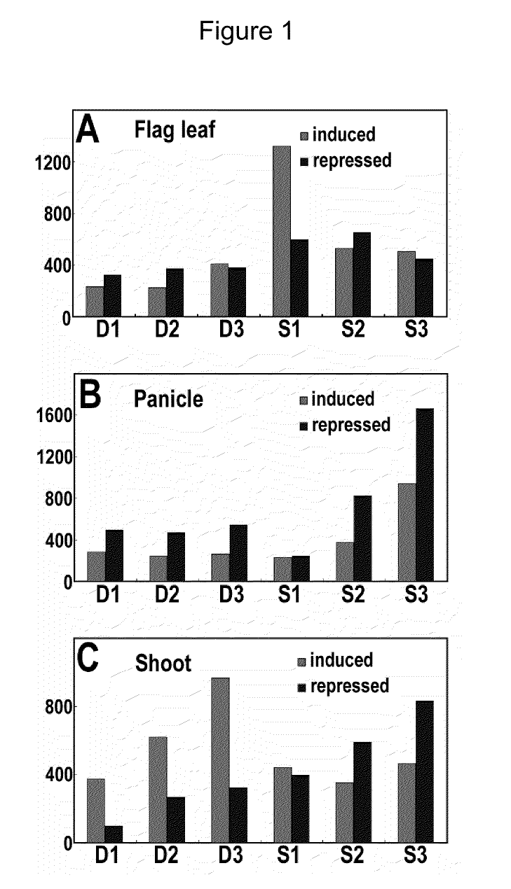

After standard processing such processing set forth in the aforementioned Ma, L. G. et al. (2005b) and Jiao, Y. et al. (2005) references, data sets from the three replicates of each stage were normalized using linear models such as those linear models described in (Smyth et al., 2005) incorporated by reference herein in its entirety. For each oligo probe, a log ratio of stress treatment sample versus unstressed sample was calculated and an FDR adjusted P value was obtained. Genes with values that passed a stringent criterion of an adjusted P value less than 0.05 and a three-fold or more change in expression were selected for further analysis. Numbers of genes up- or down-regulated by drought or by high-salinity stress at each stage in each organ were shown in FIGS. 1A-1C. In total, 582, 1,257 and 614 drought up-regulated genes and 795, 646 and 1,305 drought down-regulated genes were identified in flag leaf, shoot and panicle, respectively; and 1,676, 817 and 1,310 high-salinity up-regulated genes and 1,270, 1,323 and 2,284 high-salinity down-regulated genes were identified in flag leaf, shoot and panicle, respectively (See FIGS. 2A and 2B). The complete lists of these genes are available in the supplemental data (see Supplemental Tables 1 to 12).

Most of the previously known genes responsive to both drought and high-salinity stress have been recovered from our microarray analysis. Those include the LEA protein (OsJRFA063984), aquaporin (OsIRUAOO131 1), OsNAC1 (OsJRFA1O8O8O), dehydrin rab 16b (OsIFCC035025) and DREB1 (OsJRFAO67313). Many other responsive genes have been identified: for the first time in this study. Among those newly revealed genes, some are expected to be involved in general cell function or known to be involved in other stress responses, while others have putative or unknown function. The annotation for some of those genes suggested that they include transcription factors from multiple families, heat shock proteins, various stress (drought, high-salinity, disease, cold, and ABA) responsive genes, protein kinases, transporters, photosynthesis enzymes, and other metabolic pathways. The diversity of affected processes suggests a high level of complexity in regulation.

RNA preparation, fluorescent labeling of probes, slide hybridization, washing and scanning were performed according to the method reported in the article by Ma, L. G. et al. (2005b). Total RNA was prepared from frozen samples using the RNAwiz reagent commercially available from Ambion, Inc. of Austin, Tex., part of the Molecular Biology Division of Applied Biosystems, Foster City, Calif. Batch RNA preparation was used to generate a labeled cDNA probe for hybridization. There were at least 3 high-quality replicate data sets for each experiment, with each data set obtained from an independent biological sample.

The cDNA probes produced from the stress-treated and corresponding untreated control sample pairs were hybridized to microarray slides. For each sample point, three replicates were performed with dye-swap to correct for uneven dye effects. The cDNA synthesis, probe labeling, and microarray hybridization, microarray slide washing, and array scanning were performed according to procedures set forth in the article by Jiao, Y. et al. (2005). Hybridized microarray slides were scanned with an Axon GenePix 4000B scanner, and independent TIFF images for both Cy3 and Cy5 channels were generated for subsequent analysis.

Microarray and Initial Data Analysis

The microarray construction, microarray data quality control and normalization were conducted in accordance with procedures set forth in articles by Ma, L. G. et al. (2005b) and Jiao, Y. et al. (2005). To identify the genes that exhibited differential expression in response to drought and high salinity stress, Limma R Package was used to perform null hypothesis tests by fitting a linear model to the expression data as reported in “Limma: Linear Models for Microarray Data”, by Symth, G. K., In Bioinformatics and Computational Biology Solutions Using R and Bioconductor, pp. 397-420 (New York: Springer (2005)) incorporated by reference herein in its entirety. During pair-wise comparison between each treated sample versus a control, moderated t-statistics were computed using an empirical Bayes method to shrink the gene-wise sample variance towards a common value as reported in “Linear Models and Empirical Bayes Methods for Assessing Differential Expression in Microarray Experiments”, by Smyth, G. K., Statistical Applications in Genetics and Molecular Biology 3, Article 3 (2004), and the differential level was represented by computing the log-transformed intensity ratio. The P value adjustment used for the false discovery rate control for multiple testing employs the Benjamini and Hochberg method as discussed in “The Adaptive Control of the False Discovery Rate in Multiple Hypothesis Testing”, by Benjamini, Y. and Hochberg, Y., J. Behav. Educ. Statist. 25:60-83 (2000), and “Identifying Differentially Expressed Genes Using False Discovery Rate Controlling Procedures”, by Reiner, L. et al., Bioinformatics 19:368-375 (2003); both articles being incorporated by reference herein in their entireties. Genes were considered to have significant differences in expression level when falling within an adjusted P value threshold of 0.05, and an additional threefold strict criterion was further applied to avoid identification of false positives.

RT-PCR Analysis of Genes

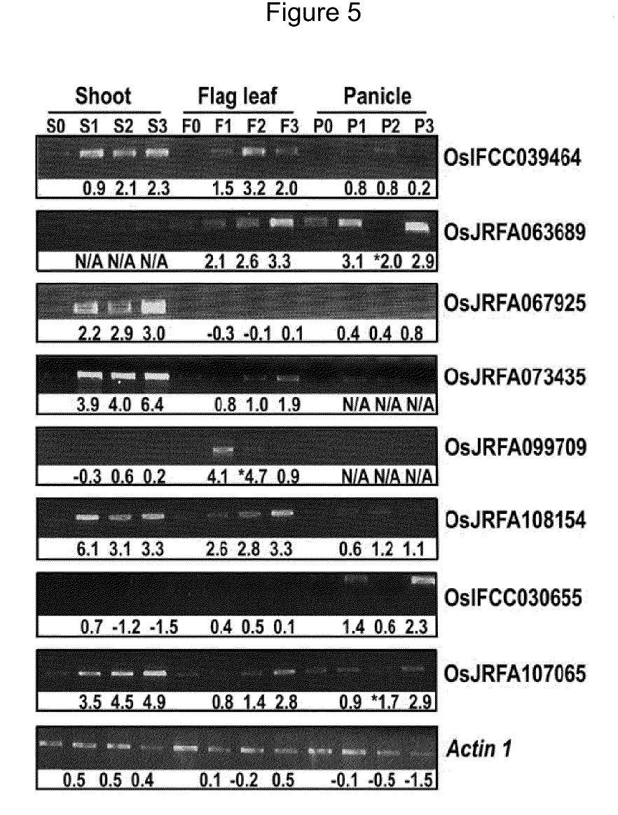

To validate the microarray data, RT-PCR analysis was used to verify the transcription response of representative genes from microarray results. For this purpose, we picked a group of eight representative genes and designed primers for RT-PCR analysis (Supplemental Table 17). The cDNA templates were synthesized from total RNA samples prepared from shoot, flag leaf and panicle under drought and unstressed controls. The semi-quantitative RT-PCR results for the eight representative genes were shown in FIG. 5. The expression patterns of all eight genes reflected changes observed by microarray analysis fairly accurately for all three organs examined, except in three cases where RT-PCR failed to confirm the microarray results (See FIG. 5), indicating that our microarray data is reliable.

The expression profiles were further quantified by RT-PCR and compared to results obtained by chip hybridization. The first strand of cDNA was generated from 1 μg of total RNA isolated independently from each sample in a 100 μl volume and 1 μl was used as template in each PCR reaction (25 cycles of 1 minute at 94° C., 1 minute at 58° C., 1 minute at 72° C.). A total of eight drought-induced genes were selected for RT-PCR analysis (the primers of these genes are listed in Supplemental Table 16). The Actin1 gene of rice was used as a control for RT-PCR experiments (forward primer, 5′-cgcagtccaagaggggtatc-3′; reverse primer, 5′-tcctggtcatagtccagggc-3′).

Analysis of Cis-Acting Regulatory Elements

Differentially expressed genes confirmed by full-length cDNA analysis were used to further elucidate component regulatory elements. After mapping the FL-cDNAs on indica rice 12 pseudo-molecules, 2 kb DNA sequences upstream from the translation start site were extracted and searched against a plant cis-regulatory database available at www.dna.affrc.go.jp/htdocs/PLACE/signalscan.html. The abundance of known ABRE core and DRE core elements in each query set were counted.

Identification of novel cis-regulatory elements was performed using the Improbizer tool (cis-Site Seeker) available at www.cse.ucsc.edu/˜kent/improbizer/, which is based on ab-initio prediction of consensus binding sites among each co-regulated gene set. The upstream 2 kb regions of co-regulated FL-eDNA genes were used for common motif searching. To evaluate the significance for each putative novel motif, an equal number of 2 kb contrast sequences were generated in each query set by randomly arranging the four nucleotides. The standard Student's t-test on motif score tables were performed, comparing query set versus contrast set. The known ABRE motif predicted by the Improbizer has a pretty high confidence level (above 0.999), and the other two novel motifs were calculated to above a 0.95 confidence level. The Motif consensus sequences were displayed by WEBLOGO program at http://weblogo.berkeley.edu.

Gel Shift Assays

Nuclear protein extracts and gel mobility shifts were performed using procedures known to one of ordinary skill in the art as described in (Gupta et al., 1998) incorporated herein by reference in its entirety. Oligomers were synthesized using the following sequences: OsJRFAO7O7 15: GCAGATACTGCAGCCAACCTCTCT,

OsJRFAO7O7 1 SM: GCAGATACTGAAGCCAACCTCTCT. The complement sequences for each oligo were also synthesized. After denaturing and annealing, the double-stranded probes were used for labeling.

TABLE 1

| Working No. | No. of treated independent lines |
|---|---|
| KT00218 | 10 |
| KT00219 | 8 |
| KT00220 | 10 |
| KT00225 | 10 |
| KT00226 | 10 |
| KT00227 | 8 |
| KT00228 | 4 |
| KT00229 | 2 |
| KT00230 | 2 |
| KT00231 | 5 |
| KT00234 | 7 |
| KT00235 | 7 |
| KT00236 | 10 |
| KT00240 | 9 |
| KT00244 | 6 |
| KT00246 | 10 |
| KT00247 | 10 |
| KT00248 | 10 |
| KT00249 | 13 |
| KT00250 | 10 |
| KT00260 | 8 |
| KT00261 | 7 |
| KT00264 | 8 |
| KT00268 | 3 |
| KT00270 | 10 |
| KT00274 | 10 |

Referring to Table 1, the numbers of treated independent transgenic lines of each construct are shown. The Genomic DNA from transgenic rice was isolated using a Genomic DNA Isolation Kit commercially available from Tian Wei Shi Dai Biotechnology Co., Beijing, China.

Amplification of Candidate Promoter and Construction of pCAMBIA1303/Inducible-Promoter Vector

The promoters were amplified by PCR using an EXTaq Kit commercially available from TaKaRa Bio Inc., Shiga, Japan. The PCR thermocycle parameters were 94° C. for 40 seconds, 55° C. for 1 minute, and 72° C. for 2 minutes for 35 cycles each. The PCR products were then gel purified and digested with Bsa I. After pCAMBIA1303 was digested with HindIII and NcoI, it was ligated with the PCR product digested with BsaI as illustrated in the map of the pCAMBIA1303/inducible-promoter vector shown below. Positive clones were sequenced by the forward primer 5′-ATCCAGACTGAATGCCCACA-3′ and reverse primer 5′-GCACCCCAGGCTTTACACTT-3′. The Ligation Kit is commercially available from Tian Wei Shi Dai Biotechnology Co. and the remaining enzymes from TaKaRa Bio Inc.

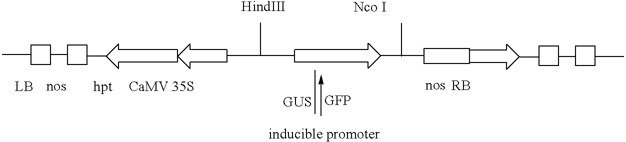

Transformation of Embryogenic Callus and Plant Regeneration

Dehulled seeds were sterilized in 1% sodium hypochlorite solution for 25 minutes, washed three times with sterilized distilled water, and then cultured on callus induction medium (N6 medium and 2 mg/L 2,4D) at 28° C. in the dark for 2 to 3 weeks. After embryogenic calli were induced from the seeds, yellowish, compact and globular calli were selected and subcultured on callus induction medium at 28° C. in the dark for 5 to 8 days. Calli were then collected and immersed into Agrobacterium cell suspension (OD600=0.10) for 20 min. After blotted dry with Whatman sterilized filter paper, Agrobacterium treated calli were transferred to co-culture medium (N6+AS 2 mg/L+2,4D 2 mg/L, pH 5.2) and grown in the dark. After co-culture, the calli were washed three times with sterilized distilled water and three times with water supplemented with 500 mg/l cefotaxime. The calli were then blotted dry, transferred to selection medium (N6+250 mg/L cefotaxime+2 mg/L 2,4D) and cultured at 28° C. in the dark. After two rounds of selection, resistant calli generated on the surface of original calli were selected, transferred to pre-differentiation medium (N6+2 mg/L ABA) and cultured at 28° C. in the dark for 10 days. White, milky and smooth calli were selected, transferred to differentiation medium (MS+2 mg/L KT+0.02 mg/L NAA+50 mg/L Hygromycin) and cultured at 28° C. under continuous cool light (>5000 1×) for 2 to 3 weeks. Shoots regenerated from resistant calli were transferred to root induction medium (MS+0.02 mg/L NAA+50 mg/L Hygromycin) for 2 weeks and plants with vigorous roots were transferred directly to a greenhouse.

Histochemical Staining of GUS Activity

Rice tissues were dissected and incubated with GUS staining solution (2.4 mM 5-bromo-4-chloro-3-indolyl b-D-glucuronic acid, 100 mM sodium phosphate (pH 7.0), 5 mM potassium ferricyanide, 0.5% Triton-X 100, 10 mM EDTA (pH 8.0), 20% methanol, and 2% DMSO) for 12 hours. The staining solution was then removed and the tissues were kept in 70% ethanol. The stained tissues were examined using a standard dissection microscope.

RT-PCR Analysis of GUS Expression Profiles

The GUS expression profiles were quantified by RT-PCR. Total RNA was prepared from frozen samples using the Plant RNA Isolation Kit commercially available from Tian Wei Shi Dai Biotechnology Co. The first strand of cDNA was generated from 1 μg of total RNA isolated independently from each sample in a 50 μl volume using the Reverse Transcription Kit commercially available from Shanhai Shenneng Bocai, and 1 μl was used as a template in each of the following PCR reactions: 29 cycles of 40 seconds each at 94° C., 29 cycles of 1 minute each at 55° C., and 29 cycles of 1 minute each at 72° C. A total of 20 constructs in transgenic rice were selected for RT-PCR analysis. The Actin1 gene of rice was used as a control for RT-PCR experiments (forward primer, 5-TCCGTGACATCAAGGAAAAG-3′; reverse primer, 5′-GATATCAACATCGCACTTCATG-3′). The GUS-GFP gene for RT-PCR experiments was used as a reporter to identify the inducible promoter (forward primer, 5′-AACTGCATCAGCCGATTATCATC-3′; reverse primer, 5′-GCATTGAACACCATAAGAGAAAGT-3′).

### RESULTS

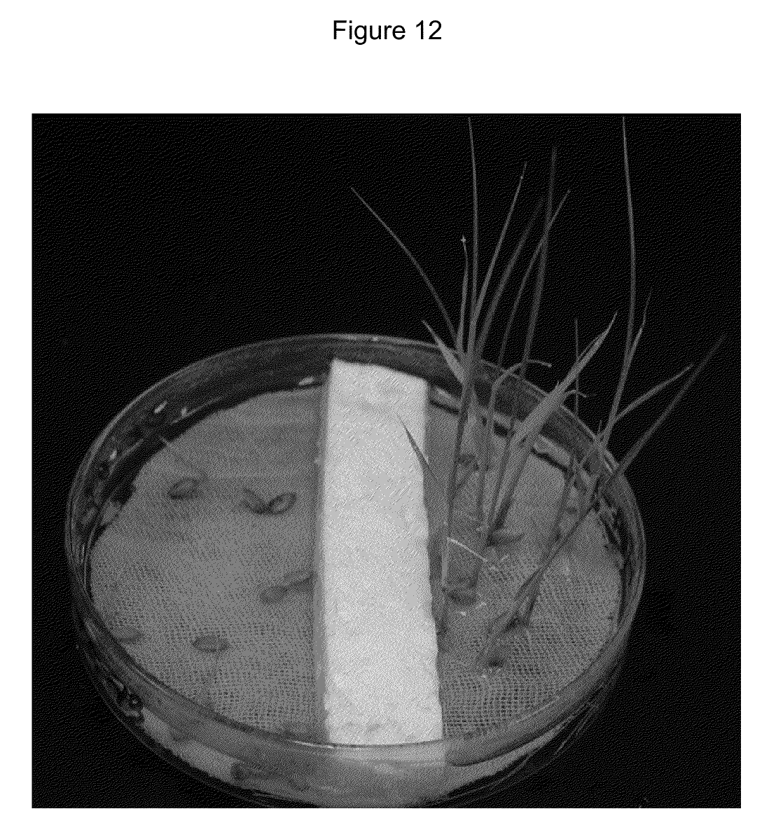

Gus staining showed that six promoters were induced by drought stress in 26 candidate constructs. The six promoters are identified by SEQ ID NOs: 1-6 provided in the Sequence Listings appended at the end of the Detailed Description. The T1 transgenic rice was selected by 50 mg/L hygromycin and then transferred in soil. Samples were treated and collected as described in the aforementioned section entitled “Plant Material and Stress Treatments” and illustrated in the photograph of FIG. 12.

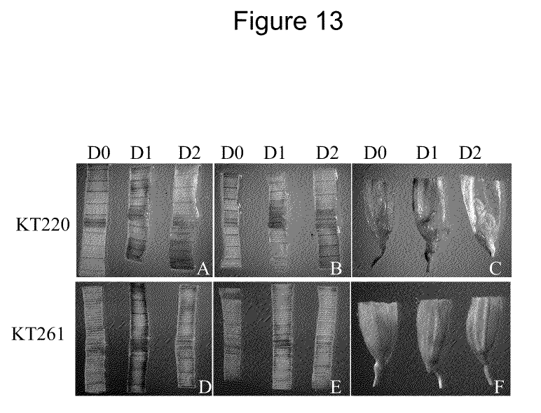

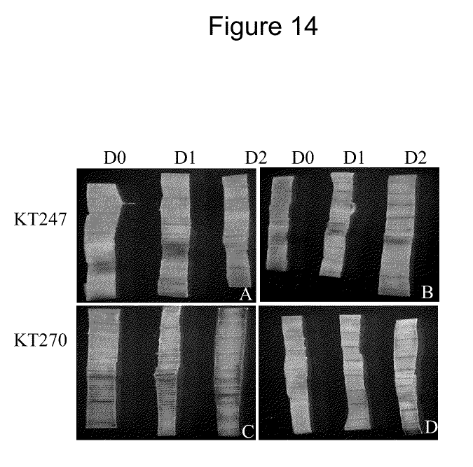

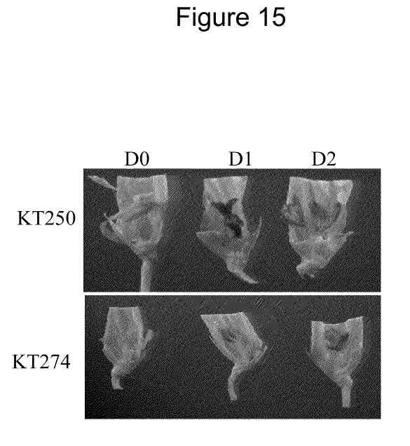

After screening 159 lines generated by transforming with 26 pCAMBIA1303/inducible-promoter vectors, 6 promoters were identified. These 6 promoters displayed drought-inducible GUS-staining in seedlings, flag leaf or panicle. GUS activity in transgenic rice KT220 and 261 was drought inducible in all tissue types, but most strongly in the vascular tissues (See FIG. 13). GUS activity in transgenic rice KT247 and 270 was drought inducible in both shoot and flag leaves, but could not be found in panicles (See FIG. 14). GUS activity in transgenic rice KT250 and 274 was drought inducible only in panicles, but could not be found in shoot and flag leaves (See FIG. 15). No GUS activity was detected in any of the remaining transgenic lines with the other 20 constructs.

The Information of the Six Promoters Selected by Gus Staining

The PCR primers, the putative gene function and length of the amplified fragments of the selected promoters are shown in Table 2 and their respective gene sequence listing are appended at the end of this disclosure.

TABLE 2

| | | | | Length |
|---|---|---|---|---|
| Working | | | | of the |
| No. | Predict gene function | 5′ Primer of the promoter | 3′ primer of the promoter | promoter |
| KT00220 | BTB/POZ domain, putative | GAGGGGTCTCAAGCTTACAGGCTTC | CCTCGGTCTCCCATGGGACGAATCG | 1301 |
| | | ACGTAGAACCAA | ACGCTTAAAGAA | |
| KT00247 | Protease inhibitor/seed | GAGGGGTCTCAAGCTTTTGGTTTTT | CCTCGGTCTCCCATGGTGGTTGGTA | 1302 |
| | storage/LTP family, putative | GACTGCCTCATC | GCTAGTGGGCTA | |
| KT00250 | putative phytochelatin | GAGGGGTCTCAAGCTTTCAACAAAA | CCTCGGTCTCCCATGGAGATGGATG | 1305 |
| | synthetase, 3′-partial | ACCATCCACTCC | TGGACGCACTAA | |
| KT00261 | hypothetical protein | GAGGGGTCTCAAGCTTTACCCGCGA | CCTCGGTCTCCCATGGGTTTGGGTG | 1409 |
| | | TAATCCCGTAA | GATGGAGGACA | |
| KT00270 | G-box binding factor 1 - maize | GAGGGGTCTCAAGCTTTCACTGGCT | CCTCGGTCTCCCATGGGCGTGAGCC | 1170 |
| | | GGCTGAAGGCTAA | TGAAATGTGAGA | |
| KT00274 | expressed protein, having | GAGGGGTCTCAAGCTTTACCACGTT | CCTCGGTCTCCCATGGTGGGTTGG | 1303 |
| | alternative splicing products | CCTCAGTCCAGT | AATTGGATTAGTG | |

RT-PCR of GUS gene showed that five promoters were obviously induced by drought stress in the 20 candidate constructs that can not be detected by GUS staining

The T1 transgenic rice was selected by 50 mg/L hygromycin and then transferred in soil. Samples were treated and collected as described in “Plant Material and Stress Treatments”. The total RNA was isolated and RT-PCR was performed as described in “RT-PCR Analysis of GUS expression profiles”.

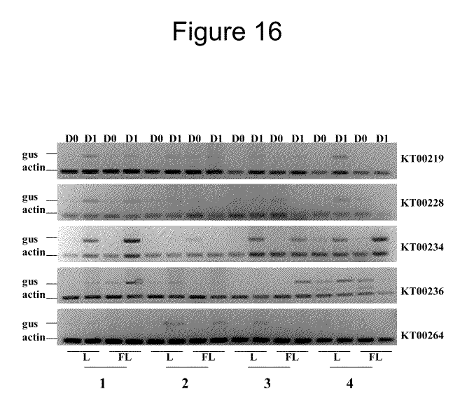

After screening 102 lines generated by transforming with the 20 pCAMBIA1303/inducible-promoter vectors, another 5 promoters were identified, which displayed drought-inducible GUS-expression in seedlings and flag leaf. These five promoters are identified by SEQ ID NOs: 7-11 in the Sequence Listings appended hereto at the end of the Detailed Description. GUS expression in transgenic rice KT234, KT236 and KT 264 displayed drought inducibility in both shoot and flag leaves, while GUS expression in transgenic rice KT219 and KT228 was induced by drought treatment only in the shoot (See FIG. 16). GUS expression could not be induced by drought in the transgenic lines using the other 15 constructs.

The PCR primers, the putative gene function and length of the amplified fragments of the selected promoters are shown in Table 3 and their respective gene sequence listings appended at the end of this disclosure.

TABLE 3

| | | | | Length |
|---|---|---|---|---|
| Working | | | | of the |
| No. | Putative gene function | 5′ primer of the promoter | 3′ primer of the promoter | promoter |
| KT00219 | expressed protein | GAGGGGTCTCAAGCTTCAGTACGTC | CCTCGGTCTCCCATGGGCTACTCGA | 1284 |
| | | GCCTATCACCAT | TCACTCCATTCG | |
| K100228 | hypothetical protein | GAGGGGTCTCAAGCTTAAGCAAGTC | CCTCGGTCTCCCATGGAGCAGAAGG | 1244 |
| | | CGAAGACCATTA | CTGACAAAGTGA | |
| KT00234 | expressed protein | GAGGGGTCTCAAGCTT | CCTCGGTCTCCCATGGACCCACTTG | 1281 |
| | | GGAGATCAAACAGGAAACAA | TCAGTGACTGCT | |
| KT00236 | germin-like protein subfamily | GAGGGGTCTCAAGCTTGATGCAGGA | CCTCGGTCTCCCATGGCGGAAGTGC | 1281 |
| | 3 member 2 precursor. | GTGCGATTAAGT | AGAGATGGTTAG | |
| | [mouse-ear cress | | | |
| KT00264 | Protein kinase domain | GAGGGGTCTCAAGCTTTGGACAGCA | CCTCGGTCTCCCATGGAGGCAAGGT | 1280 |
| | | GGCTACAGATTT | GGAACTGAAAAC | |

One or more embodiments of the present invention have been described. Nevertheless, it will be understood that various modifications may be made without departing from the spirit and scope of the invention. Accordingly, other embodiments are within the scope of the following claims.

### Supplemental Tables

SUPPLEMENTAL TABLE 1
Oligo IDs

Os000030_01
Os000082_01
Os000125_01
Os000164_01
Os000169_01
Os000174_01
Os000205_01
Os000312_01
Os000325_01
Os000422_01
Os000429_01
Os000489_01
Os000540_01
Os000597_01
Os000607_01
Os000653_01
Os000698_01
Os000757_01
Os000836_01
Os000851_01
Os000873_01
Os000942_01
Os001035_01
Os001065_01
Os001086_01
Os001164_01
Os001170_01
Os001211_01
Os001217_01
Os001234_01
Os001259_01
Os001272_01
Os001315_01
Os001377_01
Os001522_01
Os001529_01
Os001564_01
Os001620_01
Os001666_01
Os001778_01
Os001842_01
Os001870_01
Os001884_01
Os001903_01
Os001930_01
Os002027_01
Os002028_01
Os002160_01
Os002182_01
Os002353_01
Os002424_01
Os002516_02
Os002521_01
Os002729_01
Os002753_01
Os003026_01
Os003221_01
Os003284_01
Os003332_01
Os003485_01
Os003733_01
Os003759_01
Os003794_01
Os003962_02
Os004668_01
Os004794_01
Os005091_01
Os005094_01
Os005586_01
Os005987_01
Os006042_02
Os006070_01
Os006088_01
Os006138_01
Os006187_02
Os006877_01
Os007161_02
Os007196_01
Os007216_01
Os007374_01
Os007535_01
Os007764_01
Os007857_01
Os007913_02
Os008548_01
Os008555_01
Os008562_01
Os008595_01
Os008604_01
Os008629_01
Os008639_01
Os008642_01
Os008648_01
Os008691_01
Os008761_01
Os008773_01
Os009032_01
Os009047_01
Os009116_01
Os009117_01
Os009123_01
Os009211_01
Os009263_01
Os009268_01
Os009303_01
Os009390_01
Os009421_01
Os009457_01
Os009536_01
Os009544_01
Os009556_01
Os009675_01
Os009679_01
Os009685_01
Os009693_01
Os009703_01
Os009705_01
Os009744_01
Os009787_01
Os009821_01
Os009858_01
Os009929_01
Os009986_01
Os009989_01
Os010010_01
Os010042_01
Os010064_01
Os010113_01
Os010122_01
Os010145_01
Os010171_01
Os010201_01
Os010206_01
Os010280_01
Os010329_01
Os010339_01
Os010372_01
Os010486_01
Os010510_01
Os010610_01
Os010685_01
Os010789_01
Os010796_01
Os010853_01
Os010861_01
Os010863_01
Os010867_01
Os010883_01
Os010885_01
Os010894_01
Os010896_01
Os010904_01
Os010941_01
Os010959_01
Os011001_01
Os011045_01
Os011080_01
Os011359_01
Os011414_01
Os011435_01
Os011504_01
Os011653_01
Os011787_01
Os011888_01
Os011930_01
Os011972_01
Os012037_01
Os012153_01
Os012157_01
Os012160_01
Os012245_01
Os012292_01
Os012374_01
Os012479_01
Os012505_01
Os012520_01
Os012586_01
Os012623_01
Os012632_01
Os012645_01
Os012652_01
Os012696_01
Os012721_01
Os012900_01
Os012911_01
Os012925_01
Os012945_01
Os013058_01
Os013311_01
Os013341_01
Os013346_01
Os013447_01
Os013497_01
Os013659_01
Os013739_01
Os013817_01
Os013838_01
Os013885_01
Os013903_01
Os014026_01
Os014063_01
Os014133_01
Os014154_01
Os014157_01
Os014317_01
Os014442_01
Os014512_01
Os014604_01
Os014613_01
Os014634_01
Os014673_01
Os014717_01
Os014747_01
Os014761_01
Os014803_01
Os014829_01
Os014944_01
Os014962_01
Os014987_01
Os015040_01
Os015085_01
Os015136_01
Os015168_01
Os015308_01
Os015310_01
Os015556_01
Os015594_01
Os015661_01
Os015684_01
Os015742_01
Os015790_01
Os015868_01
Os015880_01
Os015902_01
Os015991_01
Os015998_01
Os016074_01
Os016252_01
Os016268_01
Os016373_01
Os016376_01
Os016458_01
Os016461_01
Os016488_01
Os016523_01
Os016536_01
Os016537_01
Os016593_01
Os016652_01
Os016676_01
Os016716_01
Os016757_01
Os016793_01
Os016882_01
Os016940_01
Os016970_01
Os017030_01
Os017086_01
Os017163_01
Os017274_01
Os017279_01
Os017290_01
Os017353_01
Os017376_01
Os017454_01
Os017691_01
Os017726_01
Os038407_01
Os038428_01
Os017990_01
Os018042_01
Os018065_01
Os018144_01
Os018147_01
Os018191_01
Os018201_01
Os018255_01
Os018279_01
Os018288_01
Os018314_01
Os018324_01
Os018381_01
Os018417_01
Os018470_01
Os018533_01
Os018757_01
Os018783_01
Os018786_01
Os018818_01
Os018863_01
Os018992_01
Os019004_01
Os019022_01
Os019114_01
Os019124_01
Os019154_01
Os019194_01
Os019289_01
Os019364_01
Os019452_01
Os019510_01
Os019625_01
Os019701_01
Os019775_01
Os019855_01
Os052203_01
Os052221_01
Os019927_01
Os020016_01
Os020077_01
Os020147_01
Os020156_01
Os020216_01
Os020299_01
Os020324_01
Os020360_01
Os020389_01
Os020488_01
Os020551_01
Os020584_01
Os020653_01
Os020661_01
Os020703_01
Os020744_01
Os020807_01
Os020923_01
Os020973_01
Os021156_01
Os021165_01
Os021218_01
Os021223_01
Os021294_01
Os021457_01
Os021523_01
Os021547_01
Os021597_01
Os021604_01
Os021615_01
Os021642_01
Os021643_01
Os021648_01
Os021705_01
Os021735_01
Os054626_01
Os054634_01
Os021815_01
Os021907_01
Os021987_01
Os021996_01
Os022116_01
Os022133_01
Os022142_01
Os022156_01
Os022167_01
Os022216_01
Os022239_01
Os022280_01
Os022309_01
Os022368_01
Os022404_01
Os022407_01
Os022431_01
Os022433_01
Os022434_01
Os022512_01
Os022544_01
Os022584_01
Os022657_01
Os022756_01
Os022829_01
Os022844_01
Os022866_01
Os022929_01
Os023001_01
Os023056_01
Os023063_01
Os023116_01
Os023200_01
Os023225_01
Os023351_01
Os023404_01
Os023465_01
Os023471_01
Os023535_01
Os023546_01
Os023619_01
Os023638_01
Os023695_01
Os023706_01
Os023709_01
Os023720_01
Os023722_01
Os023774_01
Os023781_01
Os023849_01
Os023925_01
Os024037_01
Os024101_01
Os024218_01
Os024331_01
Os024445_01
Os024506_01
Os024549_01
Os024550_01
Os024737_01
Os024813_01
Os024836_01
Os024867_01
Os024947_01
Os025638_01
Os025683_01
Os025781_01
Os025847_01
Os026287_01
Os026333_01
Os026341_01
Os026390_01
Os026758_01
Os027606_01
Os027637_01
Os027641_01
Os027819_01
Os027989_01
Os028616_01
Os028746_01
Os029023_01
Os029024_01
Os029025_01
Os029201_01
Os029446_01
Os029617_01
Os029620_01
Os029672_01
Os029761_01
Os029853_01
Os029929_01
Os030007_01
Os030046_01
Os030170_01
Os030254_01
Os030343_01
Os030354_01
Os030357_01
Os030451_01
Os030533_01
Os030717_01
Os030757_01
Os030777_01
Os030802_01
Os030831_01
Os030861_01
Os030928_01
Os030950_01
Os030977_01
Os030984_01
Os030996_01
Os031090_01
Os031145_01
Os031350_01
Os031468_01
Os031525_01
Os031800_01
Os031927_01
Os031930_01
Os031960_01
Os032027_01
Os032039_01
Os032118_01
Os032244_01
Os032248_01
Os032486_01
Os032997_01
Os033003_01
Os033086_01
Os033376_01
Os033700_01
Os033917_01
Os034414_01
Os034519_01
Os034833_01
Os035619_01
Os035938_01
Os036286_01
Os036708_01
Os037298_01
Os037651_01
Os037941_01
Os038089_01
Os038312_01
Os038951_01
Os039270_01
Os039556_01
Os040381_01
Os041647_01
Os042132_01
Os042456_01
Os042892_01
Os043307_01
Os043342_01
Os043403_01
Os044033_01
Os044205_01
Os045256_01
Os045949_01
Os046116_01
Os046888_01
Os046901_01
Os046919_01
Os050611_01
Os051126_01
Os051482_01
Os051895_01
Os051904_01
Os051919_01
Os051935_01
Os051978_01
Os051996_01
Os051997_01
Os052032_02
Os052033_01
Os052160_01
Os052175_01
Os052192_01
Os052225_01
Os052293_01
Os052361_01
Os052413_01
Os052464_01
Os052557_01
Os052593_01
Os052610_01
Os052669_01
Os052782_02
Os052801_01
Os052844_01
Os053043_01
Os053057_01
Os053136_01
Os053208_01
Os053249_01
Os053431_01
Os053510_01
Os053553_01
Os053954_01
Os054035_01
Os054085_01
Os054152_01
Os054272_01
Os054428_01
Os054495_01
Os054556_01
Os054559_01
Os054562_01
Os054564_01
Os054590_01
Os054607_01
Os054621_01
Os054640_01
Os054687_01
Os054743_01
Os054789_01
Os054867_01
Os054898_01
Os054925_01
Os054963_01
Os055103_01
Os055155_01
Os055156_01
Os055299_01
Os055319_01
Os055421_01
Os055470_01
Os055624_01
Os055638_01
Os056250_01
Os056914_02
Os057063_02
Os057318_02
Os057402_02
Os057614_02

SUPPLEMENTAL TABLE 2
Oligo ID

Os000062_01
Os000208_01
Os000271_01
Os000289_01
Os000322_01
Os000366_01
Os000380_01
Os000418_01
Os000431_01
Os000443_01
Os000445_01
Os000447_01
Os000827_01
Os000842_01
Os000994_01
Os001284_01
Os001484_01
Os001829_01
Os001958_01
Os001993_01
Os002534_01
Os004141_02
Os004223_01
Os005731_02
Os006187_02
Os006651_01
Os006683_01
Os006733_01
Os007263_01
Os007432_01
Os007493_01
Os007638_01
Os007693_01
Os008133_01
Os008213_01
Os008286_01
Os008364_01
Os008553_01
Os008608_01
Os008668_01
Os008670_01
Os008685_01
Os008844_01
Os008878_01
Os009135_01
Os009234_01
Os009280_01
Os009296_01
Os009353_01
Os009523_01
Os009532_01
Os009577_01
Os009586_01
Os009590_01
Os009724_01
Os009795_01
Os009885_01
Os009906_01
Os009983_01
Os010025_01
Os010056_01
Os010073_01
Os010210_01
Os010316_01
Os010450_01
Os010668_01
Os010812_01
Os010930_01
Os011199_01
Os011200_01
Os011349_01
Os011511_01
Os011647_01
Os011669_01
Os011696_01
Os011752_01
Os011790_01
Os011833_01
Os011874_01
Os011886_01
Os011991_01
Os012036_01
Os012086_01
Os012101_01
Os012135_01
Os012159_01
Os012162_01
Os012237_01
Os012243_01
Os012279_01
Os012359_01
Os012390_01
Os012417_01
Os012424_01
Os012427_01
Os012482_01
Os012483_01
Os012488_01
Os012502_01
Os012503_01
Os012516_01
Os012603_01
Os012617_01
Os012670_01
Os012744_01
Os012753_01
Os012765_01
Os012821_01
Os012870_01
Os012917_01
Os012921_01
Os012966_01
Os013059_01
Os013161_01
Os013283_01
Os013285_01
Os013398_01
Os013414_01
Os013419_01
Os013502_01
Os013505_01
Os013532_01
Os013645_01
Os013657_01
Os013727_01
Os013783_01
Os014014_01
Os014064_01
Os014071_01
Os014116_01
Os014200_01
Os014203_01
Os014285_01
Os014300_01
Os014330_01
Os014364_01
Os014474_01
Os014707_01
Os014740_01
Os014822_01
Os014832_01
Os014849_01
Os014856_01
Os014905_01
Os014977_01
Os014988_01
Os015012_01
Os015062_01
Os015087_01
Os015125_01
Os015227_01
Os015244_01
Os015289_01
Os015309_01
Os015340_01
Os015375_01
Os015416_01
Os015512_01
Os015534_01
Os015590_01
Os015714_01
Os015743_01
Os015839_01
Os015881_01
Os015982_01
Os015987_01
Os016032_01
Os016038_01
Os016045_01
Os016087_01
Os016097_01
Os016102_01
Os016109_01
Os016158_01
Os016159_01
Os016170_01
Os016189_01
Os016190_01
Os016201_01
Os016240_01
Os016255_01
Os016366_01
Os016542_01
Os016598_01
Os016642_01
Os016648_01
Os016838_01
Os016872_01
Os016888_01
Os016917_01
Os017034_01
Os017159_01
Os017246_01
Os017300_01
Os017317_01
Os017420_01
Os017601_01
Os017640_01
Os017654_01
Os017692_01
Os017791_01
Os017795_01
Os017796_01
Os017813_01
Os017817_01
Os017838_01
Os017847_01
Os017880_01
Os017881_01
Os017882_01
Os017884_01
Os017907_01
Os017914_01
Os017917_01
Os017986_01
Os017998_01
Os018052_01
Os018073_01
Os018084_01
Os018085_01
Os018096_01
Os018106_01
Os018107_01
Os018112_01
Os018113_01
Os018117_01
Os018141_01
Os018256_01
Os018278_01
Os018434_01
Os018472_01
Os018503_01
Os018580_01
Os018586_01
Os018689_01
Os018693_01
Os018713_01
Os018770_01
Os018804_01
Os018837_01
Os018842_01
Os018866_01
Os018868_01
Os018882_01
Os019081_01
Os019288_01
Os019290_01
Os019305_01
Os019308_01
Os019335_01
Os019336_01
Os019371_01
Os019381_01
Os019387_01
Os019391_01
Os019413_01
Os019426_01
Os019442_01
Os019475_01
Os019492_01
Os019493_01
Os019494_01
Os019499_01
Os019533_01
Os019542_01
Os019547_01
Os019597_01
Os019650_01
Os019684_01
Os019715_01
Os019791_01
Os019798_01
Os019835_01
Os019839_01
Os019910_01
Os019911_01
Os019937_01
Os019969_01
Os020012_01
Os020075_01
Os020140_01
Os020220_01
Os020300_01
Os020392_01
Os020455_01
Os020462_01
Os020472_01
Os020547_01
Os020557_01
Os020576_01
Os020627_01
Os020646_01
Os020775_01
Os020796_01
Os020797_01
Os020816_01
Os020830_01
Os020844_01
Os020897_01
Os020909_01
Os020930_01
Os020931_01
Os020992_01
Os021011_01
Os021029_01
Os021038_01
Os021058_01
Os021086_01
Os021094_01
Os021105_01
Os021107_01
Os021110_01
Os021117_01
Os021137_01
Os021161_01
Os021211_01
Os021255_01
Os021264_01
Os021274_01
Os021297_01
Os021403_01
Os021408_01
Os021472_01
Os021495_01
Os021538_01
Os021602_01
Os021630_01
Os021713_01
Os021784_01
Os021796_01
Os021797_01
Os021826_01
Os021830_01
Os021834_01
Os021870_01
Os021876_01
Os021914_01
Os021916_01
Os021973_01
Os022042_01
Os022058_01
Os022074_01
Os022181_01
Os022189_01
Os022209_01
Os022274_01
Os022290_01
Os022291_01
Os022296_01
Os022299_01
Os022390_01
Os022472_01
Os022502_01
Os022585_01
Os022609_01
Os022749_01
Os022917_01
Os022935_01
Os022993_01
Os023027_01
Os023105_01
Os023323_01
Os023457_01
Os023589_01
Os023591_01
Os023673_01
Os023726_01
Os023768_01
Os023852_01
Os023870_01
Os023901_01
Os024064_01
Os024142_01
Os024144_01
Os024147_01
Os024150_01
Os024153_01
Os024155_01
Os024156_01
Os024184_01
Os024188_01
Os024252_01
Os024255_01
Os024263_01
Os024278_01
Os024279_01
Os024335_01
Os024428_01
Os024436_01
Os024491_01
Os024555_01
Os024600_01
Os024627_01
Os024637_01
Os024681_01
Os024708_01
Os024772_01
Os024793_01
Os024803_01
Os024887_01
Os024910_01
Os024926_01
Os024932_01
Os024968_01
Os025006_01
Os025041_01
Os025081_01
Os025103_01
Os025131_01
Os025135_01
Os025147_01
Os025155_01
Os025228_01
Os025321_01
Os025330_01
Os025507_01
Os025673_01
Os025768_01
Os025814_01
Os025846_01
Os025853_01
Os025871_01
Os025882_01
Os025900_01
Os025915_01
Os025927_01
Os025930_01
Os025944_01
Os025999_01
Os026001_01
Os026008_01
Os026142_01
Os026159_01
Os026161_01
Os026190_01
Os026191_01
Os026265_01
Os026272_01
Os026335_01
Os026388_01
Os026419_01
Os026422_01
Os026432_01
Os026492_01
Os026536_01
Os026663_01
Os026780_01
Os026787_01
Os026812_01
Os026912_01
Os026920_01
Os026941_01
Os027101_01
Os027331_01
Os027379_02
Os027497_01
Os027533_01
Os027608_01
Os027647_01
Os027665_01
Os027686_01
Os027738_01
Os027769_01
Os027771_01
Os027776_01
Os027778_01
Os027789_01
Os027810_01
Os027820_01
Os027837_01
Os027843_01
Os027865_01
Os027876_01
Os027904_01
Os027919_01
Os027928_01
Os027938_01
Os028018_01
Os028041_01
Os028042_01
Os028043_01
Os028044_01
Os028049_01
Os028072_01
Os028087_01
Os028102_01
Os028113_01
Os028127_01
Os028138_01
Os028143_01
Os028163_01
Os028173_01
Os028175_01
Os028197_01
Os028234_01
Os028262_01
Os028295_01
Os028350_01
Os028369_01
Os028421_01
Os028483_01
Os028491_01
Os028492_01
Os028553_01
Os028561_01
Os028562_01
Os028573_01
Os028583_01
Os028607_01
Os028614_01
Os028621_01
Os028633_01
Os028654_01
Os028662_01
Os028679_01
Os028697_01
Os028717_01
Os028762_01
Os028793_01
Os028820_01
Os028824_01
Os028859_01
Os028874_01
Os028899_01
Os028982_01
Os028983_01
Os029013_01
Os029017_01
Os029018_01
Os029112_01
Os029118_01
Os029171_01
Os029220_01
Os029303_01
Os029315_01
Os029367_01
Os029492_01
Os029577_01
Os029619_01
Os029734_01
Os030427_01
Os030763_01
Os030776_01
Os031095_01
Os031388_01
Os031408_01
Os031418_01
Os031575_01
Os031851_01
Os032136_01
Os032172_01
Os032245_01
Os032533_01
Os032882_01
Os033585_01
Os033597_01
Os033604_01
Os034112_01
Os034449_01
Os034467_01
Os034473_01
Os034520_01
Os034644_01
Os034737_01
Os034791_01
Os034865_01
Os034905_01
Os035048_01
Os035266_01
Os035555_01
Os035699_01
Os035712_01
Os036241_01
Os036360_01
Os036576_01
Os036618_01
Os036624_01
Os036637_01
Os036678_01
Os036833_01
Os036880_01
Os037048_01
Os037269_01
Os037351_01
Os037632_01
Os037758_01
Os038150_01
Os038283_01
Os038441_01
Os038478_01
Os038648_01
Os038798_01
Os039346_01
Os039422_01
Os039495_01
Os039503_01
Os039617_01
Os039731_01
Os039737_01
Os039938_01
Os039988_01
Os040248_01
Os040338_01
Os040604_01
Os040660_01
Os041196_01
Os042032_01
Os042434_01
Os042450_01
Os042636_01
Os042768_01
Os043104_01
Os043121_01
Os043475_01
Os043501_01
Os043894_01
Os044048_01
Os044197_01
Os044246_01
Os044249_01
Os044269_01
Os044274_01
Os044300_01
Os044301_01
Os044312_01
Os044380_01
Os044530_01
Os044557_01
Os044567_01
Os044583_01
Os044587_01
Os044613_01
Os044656_01
Os044785_01
Os044797_01
Os044959_01
Os045461_01
Os045483_01
Os045565_01
Os046571_01
Os046641_01
Os046731_01
Os046825_01
Os046942_01
Os047011_01
Os047090_01
Os047309_01
Os047348_01
Os047397_01
Os047400_01
Os047450_01
Os048285_01
Os048375_01
Os051951_01
Os051956_01
Os051959_01
Os051973_01
Os051998_01
Os052052_01
Os052075_01
Os052095_01
Os052123_01
Os052145_01
Os052271_01
Os052281_01
Os052334_01
Os052370_01
Os052396_01
Os052414_01
Os052418_01
Os052419_01
Os052442_01
Os052471_01
Os052526_01
Os052569_01
Os052670_01
Os052672_01
Os052700_01
Os052713_01
Os052727_01
Os052743_01
Os052791_01
Os052826_01
Os052868_01
Os052880_01
Os052934_01
Os053172_01
Os053204_01
Os053247_01
Os053281_01
Os053353_01
Os053376_02
Os053608_01
Os053687_01
Os053713_01
Os053924_01
Os053976_01
Os053989_01
Os054160_01
Os054193_01
Os054197_01
Os054221_01
Os054282_01
Os054300_01
Os054319_01
Os054366_01
Os054377_01
Os054430_01
Os054432_01
Os054447_01
Os054452_01
Os054468_01
Os054478_01
Os054490_01
Os054493_01
Os054509_01
Os054539_01
Os054583_01
Os054627_01
Os054638_01
Os054741_01
Os054752_01
Os054779_01
Os054780_01
Os054785_01
Os054835_01
Os054890_01
Os054981_01
Os055002_01
Os055041_01
Os055046_01
Os055053_01
Os055085_01
Os055106_01
Os055132_01
Os055154_01
Os055170_01
Os055192_01
Os055200_01
Os055232_01
Os055241_01
Os055245_01
Os055250_01
Os055265_01
Os055268_01
Os055283_01
Os055345_01
Os055398_01
Os055399_01
Os055499_01
Os055529_01
Os055539_01
Os055545_01
Os055569_01
Os055635_01
Os055681_01
Os055780_01
Os055969_01
Os056132_01
Os056232_01
Os056233_01
OS056279_01
Os056301_01
Os056317_01
Os056322_01
Os056324_01
Os056342_01
Os056344_01
Os056363_01
Os056367_01
Os056439_01
Os056446_01
Os056506_01
Os056542_01
Os056583_01
Os056638_01
Os056722_01
Os056752_01
Os056792_01
Os056800_01
Os056826_01
Os056872_01
Os056919_02
Os056936_01
Os056989_02
Os057020_02
Os057198_02
Os057204_01
Os057245_02
Os057264_02
Os057340_02
Os057389_02
Os057436_02
Os057469_02
Os057666_02

SUPPLEMENTAL TABLE 3
Oligo ID

Os000014_01
Os000030_01
Os000080_01
Os000087_01
Os000174_01
Os000197_01
Os000225_01
Os000251_01
Os000253_01
Os000312_01
Os000324_01
Os000335_01
Os000340_01
Os000347_01
Os000352_01
Os000381_01
Os000393_01
Os000417_01
Os000421_01
Os000508_01
Os000532_01
Os000545_01
Os000587_01
Os000590_01
Os000597_01
Os000607_01
Os000664_01
Os000665_01
Os000669_01
Os000757_01
Os000773_01
Os000836_01
Os000881_01
Os000995_01
Os001016_01
Os001046_01
Os001087_01
Os001188_01
Os001238_01
Os001248_01
Os001272_01
Os001300_02
Os001320_01
Os001377_01
Os001497_01
Os001537_01
Os001631_01
Os001649_01
Os001657_01
Os001682_01
Os001738_01
Os001778_01
Os001806_01
Os001842_01
Os001863_01
Os001869_01
Os001870_01
Os001919_01
Os001920_01
Os001925_01
Os001930_01
Os001941_01
Os002020_01
Os002028_01
Os002150_01
Os002152_01
Os002160_01
Os002569_01
Os002678_01
Os002696_01
Os002729_01
Os002753_01
Os002858_01
Os002921_01
Os002960_02
Os003025_01
Os003223_01
Os003332_01
Os003443_01
Os003485_01
Os003568_02
Os003794_01
Os003989_01
Os004059_01
Os004143_01
Os004406_01
Os004591_01
Os004668_01
Os004720_01
Os005159_01
Os005216_01
Os005402_01
Os006070_01
Os006478_01
Os006546_02
Os006932_01
Os007068_01
Os007069_01
Os007161_02
Os007172_01
Os007189_01
Os007196_01
Os007891_01
Os008023_01
Os008400_01
Os008424_01
Os008453_01
Os008488_01
Os008495_01
Os008538_01
Os008595_01
Os008615_01
Os008629_01
Os008639_01
Os008643_01
Os008644_01
Os008648_01
Os008676_01
Os008722_01
Os008737_01
Os008761_01
Os008762_01
Os008777_01
Os008787_01
Os008889_01
Os008912_01
Os009011_01
Os009081_01
Os009091_01
Os009133_01
Os009144_01
Os009184_01
Os009222_01
Os009247_01
Os009303_01
Os009318_01
Os009325_01
Os009326_01
Os009340_01
Os009357_01
Os009415_01
Os009457_01
Os009493_01
Os009503_01
Os009504_01
Os009511_01
Os009536_01
Os009556_01
Os009588_01
Os009602_01
Os009606_01
Os009629_01
Os009661_01
Os009679_01
Os009689_01
Os009726_01
Os009739_01
Os009744_01
Os009776_01
Os009779_01
Os009798_01
Os009821_01
Os009822_01
Os009824_01
Os009847_01
Os009862_01
Os009927_01
Os009986_01
Os009991_01
Os010011_01
Os010038_01
Os010061_01
Os010064_01
Os010121_01
Os010122_01
Os010145_01
Os010157_01
Os010171_01
Os010206_01
Os010218_01
Os010236_01
Os010242_01
Os010278_01
Os010290_01
Os010313_01
Os010317_01
Os010333_01
Os010372_01
Os010376_01
Os010416_01
Os010424_01
Os010452_01
Os010486_01
Os010510_01
Os010610_01
Os010664_01
Os010685_01
Os010773_01
Os010789_01
Os010794_01
Os010828_01
Os010835_01
Os010858_01
Os010861_01
Os010862_01
Os010863_01
Os010864_01
Os010880_01
Os010885_01
Os010904_01
Os010912_01
Os010924_01
Os010927_01
Os010953_01
Os010959_01
Os011001_01
Os011039_01
Os011045_01
Os011080_01
Os011103_01
Os011247_01
Os011252_01
Os011290_01
Os011308_01
Os011311_01
Os011359_01
Os011368_01
Os011435_01
Os011532_01
Os011563_01
Os011597_01
Os011643_01
Os011645_01
Os011760_01
Os011785_01
Os011812_01
Os011870_01
Os011876_01
Os011890_01
Os011930_01
Os011937_01
Os011938_01
Os011941_01
Os011972_01
Os011973_01
Os011982_01
Os012028_01
Os012035_01
Os012037_01
Os012089_01
Os012153_01
Os012157_01
Os012160_01
Os012193_01
Os012198_01
Os012200_01
Os012259_01
Os012287_01
Os012374_01
Os012378_01
Os012415_01
Os012444_01
Os012472_01
Os012479_01
Os012497_01
Os012506_01
Os012537_01
Os012553_01
Os012586_01
Os012619_01
Os012632_01
Os012645_01
Os012660_01
Os012731_01
Os012762_01
Os012795_01
Os012816_01
Os012855_01
Os012900_01
Os012910_01
Os012927_01
Os013030_01
Os013036_01
Os013050_01
Os013085_01
Os013186_01
Os013239_01
Os013315_01
Os013346_01
Os013388_01
Os013432_01
Os013447_01
Os013469_01
Os013477_01
Os013526_01
Os013536_01
Os013609_01
Os013659_01
Os013718_01
Os013749_01
Os013772_01
Os013791_01
Os013800_01
Os013814_01
Os013838_01
Os013841_01
Os013849_01
Os013850_01
Os013914_01
Os013929_01
Os013938_01
Os013958_01
Os013986_01
Os014013_01
Os014026_01
Os014040_01
Os014060_01
Os014063_01
Os014104_01
Os014106_01
Os014108_01
Os014127_01
Os014133_01
Os014154_01
Os014157_01
Os014183_01
Os014197_01
Os014317_01
Os014327_01
Os014344_01
Os014438_01
Os014442_01
Os014467_01
Os014505_01
Os014511_01
Os014558_01
Os014585_01
Os014590_01
Os014599_01
Os014603_01
Os014604_01
Os014618_01
Os014626_01
Os014634_01
Os014661_01
Os014662_01
Os014682_01
Os014692_01
Os014749_01
Os014766_01
Os014796_01
Os014803_01
Os014810_01
Os014814_01
Os014816_01
Os014836_01
Os014856_01
Os014879_01
Os014900_01
Os014920_01
Os014963_01
Os014977_01
Os014980_01
Os015040_01
Os015073_01
Os015085_01
Os015118_01
Os015149_01
Os015152_01
Os015154_01
Os015215_01
Os015581_01
Os015589_01
Os015594_01
Os015599_01
Os015601_01
Os015612_01
Os015651_01
Os015661_01
Os015684_01
Os015694_01
Os015695_01
Os015735_01
Os015766_01
Os015790_01
Os015810_01
Os015843_01
Os015844_01
Os015860_01
Os015880_01
Os015908_01
Os015910_01
Os015916_01
Os015934_01
Os015961_01
Os015998_01
Os016030_01
Os016064_01
Os016070_01
Os016133_01
Os016159_01
Os016165_01
Os016170_01
Os016205_01
Os016214_01
Os016238_01
Os016240_01
Os016245_01
Os016249_01
Os016254_01
Os016285_01
Os016303_01
Os016305_01
Os016359_01
Os016410_01
Os016457_01
Os016461_01
Os016477_01
Os016479_01
Os016483_01
Os016486_01
Os016504_01
Os016505_01
Os016515_01
Os016527_01
Os016536_01
Os016581_01
Os016593_01
Os016599_01
Os016637_01
Os016652_01
Os016676_01
Os016687_01
Os016700_01
Os016701_01
Os016720_01
Os016744_01
Os016747_01
Os016749_01
Os016757_01
Os016793_01
Os016797_01
Os016826_01
Os016842_01
Os016871_01
Os016882_01
Os016888_01
Os016891_01
Os016892_01
Os016919_01
Os016940_01
Os016950_01
Os017003_01
Os017004_01
Os017028_01
Os017080_01
Os017116_01
Os017138_01
Os017161_01
Os017163_01
Os017165_01
Os017190_01
Os017213_01
Os017217_01
Os017225_01
Os017243_01
Os017250_01
Os017256_01
Os017258_01
Os017259_01
Os017267_01
Os017274_01
Os017290_01
Os017334_01
Os017364_01
Os017376_01
Os017379_01
Os017407_01
Os017416_01
Os017457_01
Os017459_01
Os017461_01
Os017538_01
Os017620_01
Os017653_01
Os017764_01
Os017777_01
Os017806_01
Os017813_01
Os017830_01
Os017890_01
Os017893_01
Os017939_01
Os017943_01
Os017954_01
Os017993_01
Os018042_01
Os018046_01
Os018049_01
Os018078_01
Os018092_01
Os018147_01
Os018181_01
Os018184_01
Os018196_01
Os018211_01
Os018230_01
Os018281_01
Os018314_01
Os018329_01
Os018334_01
Os018341_01
Os018362_01
Os018369_01
Os018377_01
Os018381_01
Os018409_01
Os018417_01
Os018466_01
Os018573_01
Os018682_01
Os018734_01
Os018739_01
Os018757_01
Os018783_01
Os018786_01
Os018795_01
Os018811_01
Os018830_01
Os018834_01
Os018854_01
Os018863_01
Os018876_01
Os018880_01
Os018916_01
Os018949_01
Os018953_01
Os018961_01
Os018968_01
Os019004_01
Os019022_01
Os019062_01
Os019066_01
Os019120_01
Os019124_01
Os019159_01
Os019191_01
Os019296_01
Os019299_01
Os019334_01
Os019335_01
Os019343_01
Os019350_01
Os019364_01
Os019366_01
Os019382_01
Os019400_01
Os019439_01
Os019483_01
Os019516_01
Os019540_01
Os019545_01
Os019578_01
Os019600_01
Os019627_01
Os019662_01
Os019668_01
Os019775_01
Os019785_01
Os019813_01
Os019831_01
Os019855_01
Os019868_01
Os019878_01
Os019884_01
Os019901_01
Os019923_01
Os019974_01
Os020035_01
Os020190_01
Os020194_01
Os020329_01
Os020336_01
Os020383_01
Os020389_01
Os020397_01
Os020411_01
Os020426_01
Os020427_01
Os020428_01
Os020435_01
Os020529_01
Os020551_01
Os020606_01
Os020635_01
Os020660_01
Os020703_01
Os020731_01
Os020782_01
Os020797_01
Os020828_01
Os020838_01
Os020934_01
Os020962_01
Os020973_01
Os021006_01
Os021114_01
Os021156_01
Os021165_01
Os021173_01
Os021179_01
Os021218_01
Os021311_01
Os021381_01
Os021438_01
Os021488_01
Os021507_01
Os021509_01
Os021510_01
Os021518_01
Os021580_01
Os021597_01
Os021603_01
Os021632_01
Os021692_01
Os021705_01
Os021726_01
Os021771_01
Os021884_01
Os021893_01
Os021912_01
Os021942_01
Os021980_01
Os021987_01
Os021996_01
Os022001_01
Os022021_01
Os022028_01
Os022047_01
Os022119_01
Os022131_01
Os022133_01
Os022134_01
Os022135_01
Os022142_01
Os022148_01
Os022161_01
Os022209_01
Os022217_01
Os022239_01
Os022247_01
Os022261_01
Os022265_01
Os022272_01
Os022279_01
Os022281_01
Os022291_01
Os022314_01
Os022359_01
Os022421_01
Os022433_01
Os022480_01
Os022591_01
Os022776_01
Os022829_01
Os022845_01
Os022859_01
Os022866_01
Os022876_01
Os022915_01
Os022919_01
Os022925_01
Os022927_01
Os022932_01
Os022936_01
Os022949_01
Os022955_01
Os022976_01
Os022981_01
Os022984_01
Os022986_01
Os023044_01
Os023056_01
Os023063_01
Os023117_01
Os023136_01
Os023203_01
Os023247_01
Os023350_01
Os023421_01
Os023428_01
Os023435_01
Os023455_01
Os023465_01
Os023471_01
Os023505_01
Os023508_01
Os023519_01
Os023536_01
Os023542_01
Os023546_01
Os023582_01
Os023597_01
Os023644_01
Os023695_01
Os023706_01
Os023709_01
Os023714_01
Os023740_01
Os023774_01
Os023781_01
Os023794_01
Os023799_01
Os023810_01
Os023905_01
Os023921_01
Os023931_01
Os023944_01
Os023964_01
Os023971_01
Os023972_01
Os024007_01
Os024044_01
Os024047_01
Os024083_01
Os024085_01
Os024185_01
Os024254_01
Os024260_01
Os024289_01
Os024307_01
Os024309_01
Os024354_01
Os024415_01
Os024515_01
Os024541_01
Os024550_01
Os024577_01
Os024580_01
Os024643_01
Os024652_01
Os024746_01
Os024763_01
Os024824_01
Os024867_01
Os024881_01
Os024883_01
Os024902_01
Os024934_01
Os024982_01
Os024994_01
Os025024_01
Os025047_01
Os025051_01
Os025184_01
Os025232_01
Os025709_01
Os025741_01
Os025818_01
Os025831_01
Os025848_01
Os025997_01
Os026025_01
Os026060_01
Os026110_01
Os026142_01
Os026168_01
Os026302_01
Os026332_01
Os026333_01
Os026341_01
Os026422_01
Os026465_01
Os026466_01
Os026537_01
Os026553_01
Os026674_01
Os026758_01
Os026851_01
Os027584_01
Os027591_01
Os027613_01
Os027631_01
Os027686_01
Os027821_01
Os027989_01
Os028225_01
Os028563_01
Os028636_01
Os029007_01
Os029023_01
Os029125_01
Os029182_01
Os029226_01
Os029241_01
Os029244_01
Os029407_01
Os029427_01
Os029608_01
Os029620_01
Os029666_01
Os029667_01
Os029715_01
Os029745_01
Os029927_01
Os029929_01
Os029998_01
Os030113_01
Os030148_01
Os030331_01
Os030354_01
Os030423_01
Os030499_01
Os030548_01
Os030654_01
Os030696_01
Os031074_01
Os031080_01
Os031084_01
Os031090_01
Os031218_01
Os031277_01
Os031318_01
Os031341_01
Os031815_01
Os031898_01
Os031912_01
Os032043_01
Os032115_01
Os032356_01
Os032383_01
Os032424_01
Os032461_01
Os032530_01
Os032554_01
Os032555_01
Os032662_01
Os032862_01
Os032944_01
Os033293_01
Os033442_01
Os033451_01
Os033464_01
Os033492_01
Os033607_01
Os033690_01
Os033899_01
Os033907_01
Os033928_01
Os034022_01
Os034069_01
Os034110_01
Os034381_01
Os034460_01
Os034468_01
Os034488_01
Os034499_01
Os034528_01
Os034544_01
Os034581_01
Os034833_01
Os034853_01
Os034884_01
Os034930_01
Os034983_01
Os034995_01
Os035081_01
Os035082_01
Os035232_01
Os035336_01
Os035481_01
Os035554_01
Os035819_01
Os035852_01
Os035912_01
Os035944_01
Os035984_01
Os036015_01
Os036286_01
Os036321_01
Os036478_01
Os036491_01
Os036530_01
Os036538_01
Os036573_01
Os036737_01
Os036751_01
Os036902_01
Os036962_01
Os037163_01
Os037212_01
Os037240_01
Os037254_01
Os037327_01
Os037344_01
Os037365_01
Os037611_01
Os037613_01
Os037667_01
Os037669_01
Os037692_01
Os037744_01
Os037804_01
Os037825_01
Os037914_01
Os037977_01
Os038146_01
Os038150_01
Os038244_01
Os038290_01
Os038304_01
Os038334_01
Os038407_01
Os038569_01
Os038615_01
Os038651_01
Os039696_01
Os039702_01
Os039703_01
Os039726_01
Os039909_01
Os040046_01
Os040176_01
Os040217_01
Os040241_01
Os040340_01
Os040513_01
Os040585_01
Os040595_01
Os040604_01
Os040624_01
Os040626_01
Os040687_01
Os040694_01
Os040790_01
Os040957_01
Os041003_01
Os041164_01
Os041393_01
Os041571_01
Os041655_01
Os041735_01
Os041744_01
Os042182_01
Os042246_01
Os042285_01
Os042308_01
Os042637_01
Os042866_01
Os042902_01
Os042925_01
Os042953_01
Os043039_01
Os043118_01
Os043156_01
Os043209_01
Os043235_01
Os043315_01
Os043439_01
Os043473_01
Os043506_01
Os043981_01
Os044501_01
Os044530_01
Os044544_01
Os044560_01
Os044600_01
Os044633_01
Os044836_01
Os045107_01
Os045331_01
Os045408_01
Os045472_01
Os045499_01
Os045558_01
Os045654_01
Os045704_01
Os045929_01
Os045964_01
Os046248_01
Os046324_01
Os046382_01
Os046462_01
Os046525_01
Os046599_01
Os046704_01
Os046811_01
Os046822_01
Os046888_01
Os046999_01
Os047081_01
Os047134_01
Os047919_01
Os048239_01
Os048254_01
Os048449_01
Os048604_01
Os048930_01
Os048949_01
Os048966_01
Os049031_01
Os049160_01
Os049189_01
Os049226_01
Os049360_01
Os049645_01
Os049677_01
Os049899_01
Os050065_01
Os050195_01
Os050241_01
Os050414_01
Os050611_01
Os050627_01
Os050833_01
Os051051_01
Os051257_01
Os051499_01
Os051905_01
Os051912_01
Os051933_01
Os051935_01
Os051937_01
Os051974_01
Os051997_01
Os052001_01
Os052003_01
Os052032_02
Os052033_01
Os052035_01
Os052043_01
Os052072_01
Os052079_01
Os052109_01
Os052124_01
Os052145_01
Os052160_01
Os052175_01
Os052187_01
Os052195_01
Os052198_01
Os052206_01
Os052213_01
Os052214_01
Os052221_01
Os052225_01
Os052230_01
Os052240_01
Os052241_01
Os052254_01
Os052266_01
Os052288_01
Os052294_01
Os052311_01
Os052316_01
Os052334_01
Os052350_01
Os052361_01
Os052362_01
Os052387_01
Os052400_01
Os052419_01
Os052460_01
Os052464_01
Os052483_01
Os052558_01
Os052562_01
Os052569_01
Os052593_01
Os052616_01
Os052657_01
Os052672_01
Os052689_01
Os052700_01
Os052744_02
Os052794_01
Os052816_01
Os052818_01
Os052840_01
Os052844_01
Os052865_01
Os052909_01
Os053018_01
Os053043_01
Os053057_01
Os053062_01
Os053111_01
Os053119_01
Os053136_01
Os053206_01
Os053208_01
Os053219_01
Os053316_01
Os053331_01
Os053348_01
Os053362_01
Os053503_01
Os053510_01
Os053513_01
Os053553_01
Os053556_01
Os053566_01
Os053626_01
Os053697_01
Os053744_01
Os053746_01
Os053816_01
Os053818_01
Os053906_01
Os053931_01
Os053954_01
Os054035_01
Os054037_01
Os054079_01
Os054140_02
Os054142_01
Os054270_01
Os054385_01
Os054396_01
Os054416_01
Os054428_01
Os054449_01
Os054458_01
Os054469_01
Os054483_01
Os054497_01
Os054514_02
Os054550_01
Os054553_01
Os054562_01
Os054564_01
Os054577_01
Os054590_01
Os054603_01
Os054608_01
Os054610_01
Os054634_01
Os054637_01
Os054638_01
Os054642_01
Os054643_01
Os054656_01
Os054658_01
Os054673_01
Os054682_01
Os054684_01
Os054694_01
Os054700_01
Os054704_01
Os054723_01
Os054727_01
Os054731_01
Os054741_01
Os054743_01
Os054767_01
Os054785_01
Os054789_01
Os054830_01
Os054866_01
Os054867_01
Os054882_01
Os054883_01
Os054885_01
Os054889_01
Os054903_01
Os054909_01
Os054922_01
Os054925_01
Os054951_01
Os054974_01
Os054975_01
Os054980_01
Os054996_01
Os055002_01
Os055006_01
Os055025_01
Os055027_01
Os055028_01
Os055029_01
Os055037_01
Os055052_01
Os055079_01
Os055089_01
Os055103_01
Os055126_01
Os055128_01
Os055135_01
Os055142_01
Os055171_01
Os055191_01
Os055267_01
Os055287_01
Os055301_01
Os055319_01
Os055328_01
Os055359_01
Os055421_01
Os055470_01
Os055474_01
Os055497_01
Os055504_01
Os055518_01
Os055524_01
Os055619_01
Os055630_01
Os055708_01
Os055713_01
Os055732_01
Os055763_01
Os055818_01
Os055897_01
Os055915_01
Os055962_01
Os055970_01
Os055974_01
Os056035_01
Os056093_02
Os056123_01
Os056136_02
Os056145_01
Os056220_01
Os056230_01
Os056237_01
Os056260_01
Os056322_01
Os056374_01
Os056409_02
Os056424_01
Os056446_01
Os056474_01
Os056591_01
Os056638_01
Os056700_01
Os056731_01
Os056734_01
Os056870_01
Os056905_01
Os056918_02
Os056946_01
Os056975_02
Os056998_02
Os057032_02
Os057072_02
Os057133_02
Os057278_02
Os057327_02
Os057341_02
Os057402_02
Os057433_02
Os057435_02
Os057436_02
Os057481_02
Os057491_02
Os057552_02
Os057599_02
Os057619_02
Os057659_02

SUPPLEMENTAL TABLE 4
Oligo ID

Os000002_01
Os000005_01
Os000011_01
Os000015_01
Os000040_01
Os000051_01
Os000076_01
Os000082_01
Os000086_01
Os000163_01
Os000193_01
Os000205_01
Os000208_01
Os000262_01
Os000280_01
Os000325_01
Os000333_01
Os000343_01
Os000379_01
Os000380_01
Os000403_01
Os000427_01
Os000435_01
Os000445_01
Os000871_01
Os001218_01
Os001219_01
Os001328_01
Os001345_01
Os001367_01
Os001385_01
Os001387_01
Os001553_01
Os001775_01
Os002058_01
Os002199_01
Os002356_01
Os002532_01
Os002921_01
Os002952_01
Os003126_01
Os003252_01
Os004293_01
Os004318_01
Os004503_01
Os004794_01
Os004962_01
Os005396_01
Os005707_01
Os005941_01
Os006091_01
Os006128_01
Os006134_01
Os006382_01
Os007202_01
Os007306_01
Os007431_01
Os007693_01
Os007843_01
Os008159_01
Os008192_01
Os008355_02
Os008550_01
Os008553_01
Os008657_01
Os008668_01
Os008687_01
Os008806_01
Os008864_01
Os008878_01
Os008979_01
Os009027_01
Os009058_01
Os009077_01
Os009088_01
Os009095_01
Os009126_01
Os009157_01
Os009177_01
Os009179_01
Os009280_01
Os009283_01
Os009346_01
Os009359_01
Os009403_01
Os009490_01
Os009509_01
Os009562_01
Os009577_01
Os009586_01
Os009601_01
Os009638_01
Os009724_01
Os009737_01
Os009768_01
Os009792_01
Os009825_01
Os009870_01
Os009906_01
Os010098_01
Os010186_01
Os010218_01
Os010297_01
Os010420_01
Os010430_01
Os010577_01
Os010601_01
Os010836_01
Os010911_01
Os011127_01
Os011176_01
Os011192_01
Os011197_01
Os011226_01
Os011278_01
Os011375_01
Os011396_01
Os011687_01
Os011691_01
Os011751_01
Os011814_01
Os011824_01
Os011827_01
Os011876_01
Os011884_01
Os011888_01
Os012004_01
Os012104_01
Os012154_01
Os012159_01
Os012162_01
Os012237_01
Os012243_01
Os012274_01
Os012308_01
Os012309_01
Os012324_01
Os012348_01
Os012349_01
Os012377_01
Os012378_01
Os012413_01
Os012696_01
Os012725_01
Os012743_01
Os012765_01
Os012803_01
Os012829_01
Os012843_01
Os012844_01
Os012868_01
Os012923_01
Os012942_01
Os012955_01
Os012983_01
Os013051_01
Os013161_01
Os013205_01
Os013218_01
Os013272_01
Os013354_01
Os013414_01
Os013443_01
Os013476_01
Os013505_01
Os013548_01
Os013551_01
Os013589_01
Os013616_01
Os013626_01
Os013657_01
Os013745_01
Os013799_01
Os013848_01
Os013936_01
Os013941_01
Os013981_01
Os013995_01
Os014033_01
Os014043_01
Os014048_01
Os014064_01
Os014071_01
Os014181_01
Os014189_01
Os014203_01
Os014313_01
Os014330_01
Os014353_01
Os014365_01
Os014522_01
Os014540_01
Os014557_01
Os014686_01
Os014720_01
Os014822_01
Os014942_01
Os014962_01
Os014983_01
Os015087_01
Os015125_01
Os015150_01
Os015236_01
Os015438_01
Os015552_01
Os015625_01
Os015642_01
Os015658_01
Os015681_01
Os015713_01
Os015742_01
Os015762_01
Os015768_01
Os015786_01
Os015832_01
Os015959_01
Os016024_01
Os016057_01
Os016333_01
Os016404_01
Os016472_01
Os016493_01
Os016615_01
Os016636_01
Os016681_01
Os016693_01
Os016706_01
Os016762_01
Os016857_01
Os016858_01
Os016868_01
Os017100_01
Os017283_01
Os017310_01
Os017311_01
Os017445_01
Os017450_01
Os017466_01
Os017477_01
Os017555_01
Os017638_01
Os017649_01
Os017714_01
Os017816_01
Os017944_01
Os018070_01
Os018102_01
Os018134_01
Os018168_01
Os018176_01
Os018193_01
Os018226_01
Os018253_01
Os018264_01
Os018267_01
Os018284_01
Os018355_01
Os018365_01
Os018367_01
Os018393_01
Os018397_01
Os018473_01
Os018481_01
Os018514_01
Os018554_01
Os018557_01
Os018622_01
Os018638_01
Os018706_01
Os018787_01
Os018926_01
Os018929_01
Os019065_01
Os019078_01
Os019089_01
Os019155_01
Os019381_01
Os019426_01
Os019441_01
Os019445_01
Os019469_01
Os019493_01
Os019620_01
Os019650_01
Os019704_01
Os019728_01
Os019761_01
Os019776_01
Os019910_01
Os019947_01
Os019961_01
Os020095_01
Os020227_01
Os020253_01
Os020385_01
Os020424_01
Os020455_01
Os020556_01
Os020558_01
Os020576_01
Os020588_01
Os020627_01
Os020750_01
Os020761_01
Os020897_01
Os021017_01
Os021032_01
Os021189_01
Os021330_01
Os021435_01
Os021457_01
Os021461_01
Os021494_01
Os021649_01
Os021670_01
Os021903_01
Os021973_01
Os022055_01
Os022079_01
Os022084_01
Os022098_01
Os022120_01
Os022130_01
Os022134_01
Os022135_01
Os022145_01
Os022259_01
Os022636_01
Os022709_01
Os022766_01
Os022848_01
Os022932_01
Os023116_01
Os023194_01
Os023503_01
Os023775_01
Os023870_01
Os023873_01
Os023934_01
Os023940_01
Os024077_01
Os024139_01
Os024172_01
Os024188_01
Os024201_01
Os024233_01
Os024252_01
Os024283_01
Os024310_01
Os024409_01
Os024624_01
Os024722_01
Os024733_01
Os024783_01
Os024793_01
Os024812_01
Os024869_01
Os025118_01
Os025119_01
Os025124_01
Os025506_01
Os025761_01
Os025882_01
Os026129_01
Os026233_01
Os026253_01
Os026407_01
Os026451_01
Os026822_01
Os027119_01
Os027312_01
Os027383_01
Os027739_01
Os027757_01
Os027769_01
Os027818_01
Os027854_01
Os027925_01
Os027949_01
Os028071_01
Os028169_01
Os028179_01
Os028215_01
Os028233_01
Os028445_01
Os028580_01
Os028587_01
Os028612_01
Os028722_01
Os028897_01
Os028899_01
Os028903_01
Os029002_01
Os029013_01
Os029018_01
Os029090_01
Os029111_01
Os029178_01
Os029192_01
Os029208_01
Os029263_01
Os029327_01
Os029403_01
Os029685_01
Os029695_01
Os029755_01
Os030093_01
Os030173_01
Os030187_01
Os030320_01
Os030427_01
Os030523_01
Os030763_01
Os030865_01
Os031192_01
Os031321_01
Os031383_01
Os031414_01
Os031550_01
Os031596_01
Os031713_01
Os031730_01
Os031783_01
Os031851_01
Os032135_01
Os032161_01
Os032316_01
Os032703_01
Os032788_01
Os032798_01
Os032918_01
Os032958_01
Os033533_01
Os033577_01
Os033666_01
Os033672_01
Os033751_01
Os033789_01
Os033936_01
Os033953_01
Os034052_01
Os034095_01
Os034173_01
Os034201_01
Os034202_01
Os034203_01
Os034323_01
Os034398_01
Os034520_01
Os034610_01
Os034717_01
Os034869_01
Os034884_01
Os035048_01
Os035060_01
Os035100_01
Os035121_01
Os035255_01
Os035306_01
Os035327_01
Os035535_01
Os035571_01
Os035628_01
Os035714_01
Os035946_01
Os035985_01
Os036283_01
Os036311_01
Os036560_01
Os036569_01
Os036576_01
Os036722_01
Os036739_01
Os036751_01
Os036911_01
Os037090_01
Os037178_01
Os037234_01
Os037254_01
Os037264_01
Os037358_01
Os037671_01
Os037756_01
Os037892_01
Os037922_01
Os038332_01
Os038399_01
Os038502_01
Os038589_01
Os038619_01
Os038899_01
Os039099_01
Os039104_01
Os039345_01
Os039346_01
Os039391_01
Os039430_01
Os039432_01
Os039486_01
Os039689_01
Os039725_01
Os039730_01
Os039737_01
Os039868_01
Os040099_01
Os040266_01
Os040272_01
Os040317_01
Os040938_01
Os040960_01
Os040974_01
Os041016_01
Os041060_01
Os041647_01
Os041666_01
Os041932_01
Os042064_01
Os042345_01
Os042450_01
Os042746_01
Os042794_01
Os042919_01
Os042980_01
Os042998_01
Os043410_01
Os043572_01
Os043619_01
Os043894_01
Os044173_01
Os044465_01
Os044679_01
Os045149_01
Os045677_01
Os045876_01
Os045934_01
Os045979_01
Os046144_01
Os046151_01
Os046196_01
Os046472_01
Os046825_01
Os047357_01
Os047423_01
Os047662_01
Os047679_01
Os047794_01
Os047974_01
Os048165_01
Os048198_01
Os048269_01
Os048345_01
Os048666_01
Os048995_01
Os049037_01
Os049270_01
Os049316_01
Os049571_01
Os049821_01
Os050182_01
Os050799_01
Os050879_01
Os051128_01
Os051240_01
Os051250_01
Os051413_01
Os051895_01
Os051902_01
Os051940_01
Os051959_01
Os051978_01
Os051996_01
Os052036_01
Os052053_01
Os052054_01
Os052084_01
Os052103_01
Os052118_01
Os052124_01
Os052207_01
Os052241_01
Os052302_01
Os052303_01
Os052396_01
Os052404_01
Os052484_01
Os052531_01
Os052641_01
Os052644_01
Os052734_01
Os052747_01
Os052776_01
Os052934_01
Os053035_01
Os053073_01
Os053313_02
Os053351_01
Os053368_01
Os053489_01
Os053633_01
Os053705_01
Os053989_01
Os054106_01
Os054221_01
Os054237_01
Os054284_01
Os054375_01
Os054377_01
Os054451_01
Os054468_01
Os054487_01
Os054509_01
Os054510_01
Os054555_01
Os054601_01
Os054607_01
Os054815_01
Os054910_01
Os054914_01
Os054937_01
Os054975_01
Os055184_01
Os055224_01
Os055247_01
Os055332_01
Os055447_01
Os055571_01
Os055970_01
Os055988_01
Os056170_01
Os056437_01
Os056614_01
Os056746_01
Os056751_01
Os056762_01
Os056821_01
Os056913_02
Os056919_02
Os056920_02
Os056974_02
Os057098_02
Os057165_02
Os057254_02
Os057284_02
Os057291_02
Os057321_02
Os057469_02

SUPPLEMENTAL TABLE 5
Oligo ID

Os000087_01
Os000116_01
Os000164_01
Os000169_01
Os000213_01
Os000324_01
Os000351_01
Os000431_01
Os000485_01
Os000532_01
Os000618_01
Os000689_01
Os000787_01
Os000812_01
Os000896_01
Os000918_01
Os000959_01
Os000969_01
Os001064_01
Os001072_01
Os001111_01
Os001217_01
Os001315_01
Os001323_01
Os001332_01
Os001355_01
Os001559_01
Os001623_01
Os001666_01
Os001836_01
Os001896_01
Os001903_01
Os001927_01
Os001934_01
Os001957_01
Os001972_01
Os002152_01
Os002199_01
Os002231_01
Os002448_01
Os002518_01
Os002633_01
Os002698_01
Os002761_01
Os002858_01
Os002864_01
Os002881_01
Os002988_01
Os003284_01
Os003332_01
Os003427_01
Os003645_01
Os003956_02
Os004117_01
Os004407_01
Os005159_01
Os006964_02
Os007216_01
Os007472_01
Os007764_01
Os007907_01
Os008023_01
Os008400_01
Os008424_01
Os008433_01
Os008476_01
Os008550_01
Os008598_01
Os008604_01
Os008611_01
Os008639_01
Os008676_01
Os008690_01
Os008691_01
Os008749_01
Os008761_01
Os008768_01
Os008797_01
Os008895_01
Os008953_01
Os008954_01
Os009074_01
Os009117_01
Os009166_01
Os009359_01
Os009505_01
Os009520_01
Os009767_01
Os009858_01
Os009895_01
Os009984_01
Os009986_01
Os009998_01
Os010076_01
Os010095_01
Os010143_01
Os010162_01
Os010175_01
Os010201_01
Os010218_01
Os010224_01
Os010297_01
Os010300_01
Os010339_01
Os010372_01
Os010381_01
Os010430_01
Os010486_01
Os010515_01
Os010610_01
Os010664_01
Os010738_01
Os010789_01
Os010791_01
Os010861_01
Os010867_01
Os010904_01
Os010912_01
Os010948_01
Os010959_01
Os010961_01
Os011001_01
Os011020_01
Os011357_01
Os011371_01
Os011414_01
Os011486_01
Os011693_01
Os011838_01
Os011930_01
Os011982_01
Os012041_01
Os012157_01
Os012200_01
Os012245_01
Os012313_01
Os012479_01
Os012562_01
Os012623_01
Os012665_01
Os012683_01
Os012743_01
Os012781_01
Os012843_01
Os012865_01
Os012974_01
Os013026_01
Os013058_01
Os013186_01
Os013548_01
Os013596_01
Os013669_01
Os013696_01
Os013739_01
Os013746_01
Os013775_01
Os013849_01
Os013908_01
Os014044_01
Os014084_01
Os014107_01
Os014157_01
Os014464_01
Os014704_01
Os014747_01
Os014780_01
Os015017_01
Os015069_01
Os015134_01
Os015150_01
Os015152_01
Os015303_01
Os015308_01
Os015344_01
Os015556_01
Os015589_01
Os015592_01
Os015658_01
Os015661_01
Os015666_01
Os015687_01
Os015740_01
Os015790_01
Os015865_01
Os015880_01
Os015934_01
Os015998_01
Os016052_01
Os016085_01
Os016205_01
Os016225_01
Os016447_01
Os016652_01
Os016695_01
Os016699_01
Os016793_01
Os016841_01
Os016914_01
Os016922_01
Os017029_01
Os017109_01
Os017163_01
Os017190_01
Os017234_01
Os017294_01
Os017376_01
Os017388_01
Os017416_01
Os017443_01
Os017458_01
Os017523_01
Os017586_01
Os017728_01
Os017741_01
Os017874_01
Os018056_01
Os018065_01
Os018071_01
Os018087_01
Os018191_01
Os018194_01
Os018266_01
Os018314_01
Os018321_01
Os018362_01
Os018538_01
Os018729_01
Os018796_01
Os018867_01
Os019041_01
Os019065_01
Os019112_01
Os019114_01
Os019284_01
Os019285_01
Os019289_01
Os019402_01
Os019423_01
Os019495_01
Os019512_01
Os019648_01
Os019652_01
Os019708_01
Os019825_01
Os019992_01
Os020005_01
Os020190_01
Os020216_01
Os020217_01
Os020225_01
Os020253_01
Os020383_01
Os020500_01
Os020529_01
Os020575_01
Os020584_01
Os020629_01
Os020679_01
Os020769_01
Os021069_01
Os021072_01
Os021165_01
Os021283_01
Os021547_01
Os021615_01
Os021643_01
Os021669_01
Os021739_01
Os021804_01
Os021880_01
Os021985_01
Os022009_01
Os022135_01
Os022239_01
Os022309_01
Os022377_01
Os022404_01
Os022433_01
Os022512_01
Os022515_01
Os022617_01
Os022638_01
Os022735_01
Os022742_01
Os022831_01
Os022866_01
Os022879_01
Os022954_01
Os023013_01
Os023044_01
Os023048_01
Os023165_01
Os023351_01
Os023426_01
Os023453_01
Os023677_01
Os023707_01
Os023709_01
Os023737_01
Os023746_01
Os023781_01
Os023876_01
Os023972_01
Os023979_01
Os023988_01
Os024290_01
Os024331_01
Os024645_01
Os024798_01
Os024827_01
Os024971_01
Os025005_01
Os025027_01
Os025129_01
Os025193_01
Os025727_01
Os025801_01
Os025874_01
Os025952_01
Os026082_01
Os026287_01
Os026417_01
Os026608_01
Os027105_01
Os027596_01
Os027640_01
Os027641_01
Os027841_01
Os028381_01
Os028405_01
Os028683_01
Os028927_01
Os029109_01
Os029147_01
Os029213_01
Os029291_01
Os029410_01
Os029446_01
Os029534_01

SUPPLEMENTAL TABLE 6
Oligo ID
Os000382_01
Os000418_01
Os000443_01
Os001284_01
Os001484_01
Os001831_01
Os002648_01
Os002827_01
Os003091_01
Os003578_01
Os005103_01
Os006105_01
Os006291_01
Os006301_01
Os006743_01
Os006805_01
Os006971_02
Os007128_01
Os008176_01
Os008475_01
Os008481_01
Os008487_01
Os008640_01
Os008706_01
Os008775_01
Os009210_01
Os009326_01
Os009347_01
Os009448_01
Os009452_01
Os009557_01
Os009581_01
Os009590_01
Os009633_01
Os009678_01
Os009835_01
Os009843_01
Os009848_01
Os009870_01
Os009970_01
Os010128_01
Os010562_01
Os011121_01
Os011157_01
Os011163_01
Os011226_01
Os011280_01
Os011372_01
Os011429_01
Os011478_01
Os011527_01
Os011534_01
Os011561_01
Os011581_01
Os011719_01
Os011752_01
Os011767_01
Os011811_02
Os011871_01
Os011929_01
Os011937_01
Os011958_01
Os012011_01
Os012117_01
Os012159_01
Os012179_01
Os012193_01
Os012194_01
Os012205_01
Os012274_01
Os012321_01
Os012382_01
Os012388_01
Os012400_01
Os012409_01
Os012481_01
Os012495_01
Os012533_01
Os012588_01
Os012603_01
Os012636_01
Os012664_01
Os012685_01
Os012864_01
Os012870_01
Os012917_01
Os013123_01
Os013201_01
Os013313_01
Os013398_01
Os013460_01
Os013502_01
Os013546_01
Os013582_01
Os013645_01
Os013647_01
Os013659_01
Os013706_01
Os013845_01
Os013992_01
Os014071_01
Os014255_01
Os014281_01
Os014285_01
Os014420_01
Os014474_01
Os014488_01
Os014551_01
Os014615_01
Os014714_01
Os014832_01
Os014836_01
Os014842_01
Os014856_01
Os014865_01
Os014948_01
Os014953_01
Os014983_01
Os014988_01
Os015047_01
Os015048_01
Os015062_01
Os015063_01
Os015075_01
Os015080_01
Os015085_01
Os015197_01
Os015256_01
Os015342_01
Os015420_01
Os015494_01
Os015522_01
Os015578_01
Os015580_01
Os015588_01
Os015618_01
Os015672_01
Os015681_01
Os015696_01
Os015702_01
Os015732_01
Os015737_01
Os015746_01
Os015787_01
Os015791_01
Os015828_01
Os015885_01
Os015897_01
Os015936_01
Os015963_01
Os015982_01
Os015989_01
Os015999_01
Os016001_01
Os016021_01
Os016063_01
Os016076_01
Os016081_01
Os016091_01
Os016109_01
Os016112_01
Os016120_01
Os016124_01
Os016137_01
Os016150_01
Os016161_01
Os016164_01
Os016221_01
Os016246_01
Os016262_01
Os016269_01
Os016298_01
Os016352_01
Os016402_01
Os016404_01
Os016406_01
Os016409_01
Os016424_01
Os016440_01
Os016463_01
Os016468_01
Os016480_01
Os016507_01
Os016518_01
Os016566_01
Os016598_01
Os016644_01
Os016664_01
Os016667_01
Os016669_01
Os016687_01
Os016692_01
Os016714_01
Os016741_01
Os016747_01
Os016787_01
Os016790_01
Os016802_01
Os016818_01
Os016911_01
Os016919_01
Os016927_01
Os017034_01
Os017093_01
Os017103_01
Os017120_01
Os017140_01
Os017148_01
Os017173_01
Os017221_01
Os017246_01
Os017262_01
Os017287_01
Os017289_01
Os017301_01
Os017317_01
Os017431_01
Os017439_01
Os017440_01
Os017466_01
Os017553_01
Os017555_01
Os017640_01
Os017698_01
Os017744_01
Os017763_01
Os017770_01
Os017790_01
Os017791_01
Os017794_01
Os017805_01
Os017822_01
Os017825_01
Os017827_01
Os017851_01
Os017897_01
Os017908_01
Os017917_01
Os017998_01
Os018026_01
Os018096_01
Os018123_01
Os018130_01
Os018151_01
Os018152_01
Os018221_01
Os018232_01
Os018245_01
Os018247_01
Os018256_01
Os018287_01
Os018307_01
Os018322_01
Os018330_01
Os018360_01
Os018361_01
Os018453_01
Os018472_01
Os018532_01
Os018538_01
Os018539_01
Os018626_01
Os018631_01
Os018644_01
Os018647_01
Os018689_01
Os018693_01
Os018731_01
Os018735_01
Os018750_01
Os018756_01
Os018775_01
Os018779_01
Os018833_01
Os018834_01
Os018862_01
Os018869_01
Os018910_01
Os018928_01
Os019013_01
Os019051_01
Os019070_01
Os019072_01
Os019081_01
Os019110_01
Os019130_01
Os019167_01
Os019192_01
Os019217_01
Os019234_01
Os019235_01
Os019300_01
Os019306_01
Os019312_01
Os019356_01
Os019367_01
Os019381_01
Os019382_01
Os019395_01
Os019407_01
Os019410_01
Os019476_01
Os019482_01
Os019486_01
Os019487_01
Os019539_01
Os019574_01
Os019596_01
Os019597_01
Os019666_01
Os019723_01
Os019771_01
Os019789_01
Os019798_01
Os019810_01
Os019816_01
Os019828_01
Os019835_01
Os019845_01
Os019909_01
Os019952_01
Os019953_01
Os019968_01
Os020033_01
Os020036_01
Os020220_01
Os020237_01
Os020243_01
Os020272_01
Os020301_01
Os020344_01
Os020423_01
Os020446_01
Os020455_01
Os020457_01
Os020466_01
Os020469_01
Os020501_01
Os020505_01
Os020507_01
Os020592_01
Os020603_01
Os020627_01
Os020635_01
Os020694_01
Os020817_01
Os020818_01
Os020836_01
Os020937_01
Os020948_01
Os020966_01
Os020970_01
Os021006_01
Os021017_01
Os021042_01
Os021105_01
Os021110_01
Os021145_01
Os021167_01
Os021193_01
Os021198_01
Os021320_01
Os021334_01
Os021338_01
Os021344_01
Os021352_01
Os021373_01
Os021400_01
Os021409_01
Os021441_01
Os021471_01
Os021473_01
Os021490_01
Os021509_01
Os021534_01
Os021631_01
Os021650_01
Os021663_01
Os021671_01
Os021743_01
Os021744_01
Os021758_01
Os021792_01
Os021803_01
Os021830_01
Os021854_01
Os021861_01
Os021864_01
Os021875_01
Os021888_01
Os021892_01
Os021916_01
Os021918_01
Os021920_01
Os021929_01
Os021947_01
Os021973_01
Os021979_01
Os022007_01
Os022021_01
Os022024_01
Os022031_01
Os022032_01
Os022041_01
Os022042_01
Os022058_01
Os022084_01
Os022113_01
Os022118_01
Os022135_01
Os022151_01
Os022190_01
Os022211_01
Os022228_01
Os022247_01
Os022281_01
Os022320_01
Os022424_01
Os022476_01
Os022536_01
Os022562_01
Os022632_01
Os022636_01
Os022637_01
Os022650_01
Os022654_01
Os022695_01
Os022700_01
Os022712_01
Os022717_01
Os022720_01
Os022733_01
Os022744_01
Os022762_01
Os022791_01
Os022795_01
Os022812_01
Os022841_01
Os022851_01
Os022871_01
Os022965_01
Os023032_01
Os023116_01
Os023199_01
Os023228_01
Os023247_01
Os023277_01
Os023286_01
Os023519_01
Os023539_01
Os023541_01
Os023549_01
Os023552_01
Os023589_01
Os023803_01
Os023831_01
Os023880_01
Os024055_01
Os024110_01
Os024181_01
Os024189_01
Os024235_01
Os024269_01
Os024274_01
Os024276_01
Os024282_01
Os024295_01
Os024306_01
Os024344_01
Os024428_01
Os024466_01
Os024478_01
Os024540_01
Os024595_01
Os024612_01
Os024613_01
Os024621_01
Os024623_01
Os024625_01
Os024631_01
Os024640_01
Os024678_01
Os024679_01
Os024683_01
Os024690_01
Os024694_01
Os024728_01
Os024733_01
Os024740_01
Os024742_01
Os024754_01
Os024773_01
Os024774_01
Os024779_01
Os024784_01
Os024793_01
Os024795_01
Os024806_01
Os024814_01
Os024822_01
Os024835_01
Os024845_01
Os024885_01
Os024900_01
Os024905_01
Os024928_01
Os024930_01
Os024937_01
Os024944_01
Os024969_01
Os024972_01
Os024996_01
Os024998_01
Os025005_01
Os025006_01
Os025079_01
Os025082_01
Os025086_01
Os025113_01
Os025150_01
Os025164_01
Os025200_01
Os025214_01
Os025232_01
Os025244_01
Os025321_01
Os025338_01
Os025351_01
Os025452_01
Os025479_01
Os025525_01
Os025531_01
Os025584_01
Os025614_01
Os025701_01
Os025746_01
Os025809_01
Os025819_01
Os025822_01
Os025846_01
Os025860_01
Os025862_01
Os025886_01
Os025915_01
Os025938_01
Os025939_01
Os025961_01
Os025984_01
Os025990_01
Os026021_01
Os026022_01
Os026026_01
Os026089_01
Os026100_01
Os026148_01
Os026156_01
Os026170_01
Os026219_01
Os026239_01
Os026265_01
Os026282_01
Os026296_01
Os026311_01
Os026336_01
Os026365_01
Os026373_01
Os026394_01
Os026406_01
Os026454_01
Os026461_01
Os026467_01
Os026495_01
Os026498_01
Os026553_01
Os026564_01
Os026569_01
Os026579_01
Os026630_01
Os026666_01
Os026787_01
Os026834_01
Os026848_01
Os026858_01
Os026865_01
Os026952_01
Os027170_01
Os027194_01
Os027215_01
Os027267_01
Os027335_01
Os027379_02
Os027389_01
Os027431_01
Os027449_01
Os027455_01
Os027527_01
Os027568_01
Os027575_01
Os027579_01
Os027599_01
Os027606_01
Os027615_01
Os027663_01
Os027682_01
Os027683_01
Os027691_01
Os027694_01
Os027720_01
Os027721_01
Os027742_01
Os027743_01
Os027746_01
Os027783_01
Os027802_01
Os027808_01
Os027815_01
Os027827_01
Os027828_01
Os027842_01
Os027854_01
Os027855_01
Os027883_01
Os027884_01
Os027885_01
Os027907_01
Os027919_01
Os027938_01
Os027941_01
Os027960_01
Os027963_01
Os027995_01
Os028018_01
Os028033_01
Os028045_01
Os028092_01
Os028112_01
Os028123_01
Os028131_01
Os028157_01
Os028187_01
Os028200_01
Os028202_01
Os028206_01
Os028222_01
Os028233_01
Os028234_01
Os028247_01
Os028272_01
Os028310_01
Os028325_01
Os028326_01
Os028331_01
Os028333_01
Os028337_01
Os028345_01
Os028350_01
Os028365_01
Os028393_01
Os028436_01
Os028437_01
Os028442_01
Os028446_01
Os028455_01
Os028459_01
Os028515_01
Os028539_01
Os028544_01
Os028548_01
Os028583_01
Os028616_01
Os028620_01
Os028625_01
Os028626_01
Os028630_01
Os028631_01
Os028637_01
Os028739_01
Os028745_01
Os028755_01
Os028757_01
Os028763_01
Os028767_01
Os028787_01
Os028792_01
Os028809_01
Os028838_01
Os028849_01
Os028850_01
Os028856_01
Os028866_01
Os028874_01
Os028885_01
Os028886_01
Os028891_01
Os028914_01
Os028915_01
Os028922_01
Os028929_01
Os028933_01
Os028935_01
Os028971_01
Os028989_01
Os028998_01
Os029007_01
Os029011_01
Os029013_01
Os029014_01
Os029017_01
Os029035_01
Os029118_01
Os029136_01
Os029172_01
Os029180_01
Os029200_01
Os029216_01
Os029220_01
Os029232_01
Os029268_01
Os029314_01
Os029421_01
Os029467_01
Os029489_01
Os029492_01
Os029694_01
Os029870_01
Os030139_01
Os030207_01
Os030233_01
Os030254_01
Os030357_01
Os030721_01
Os030755_01
Os030763_01
Os030807_01
Os030808_01
Os030978_01
Os031214_01
Os031583_01
Os031608_01
Os031736_01
Os032064_01
Os032067_01
Os032123_01
Os032199_01
Os032388_01
Os032657_01
Os032863_01
Os033004_01
Os033248_01
Os033297_01
Os033438_01
Os033445_01
Os033501_01
Os033602_01
Os033612_01
Os033645_01
Os033673_01
Os033694_01
Os033703_01
Os033810_01
Os033909_01
Os034093_01
Os034299_01
Os034302_01
Os034329_01
Os034340_01
Os034443_01
Os034449_01
Os034452_01
Os034467_01
Os034491_01
Os034547_01
Os034581_01
Os034737_01
Os034776_01
Os034819_01
Os034876_01
Os034972_01
Os035012_01
Os035119_01
Os035141_01
Os035151_01
Os035159_01
Os035189_01
Os035233_01
Os035272_01
Os035391_01
Os035426_01
Os035555_01
Os035712_01
Os035804_01
Os035860_01
Os035879_01
Os035885_01
Os035900_01
Os035907_01
Os035918_01
Os036048_01
Os036241_01
Os036484_01
Os036576_01
Os036587_01
Os036618_01
Os036816_01
Os036934_01
Os037014_01
Os037031_01
Os037232_01
Os037234_01
Os037249_01
Os037450_01
Os037454_01
Os037565_01
Os037695_01
Os037720_01
Os037803_01
Os037805_01
Os037878_01
Os037882_01
Os037918_01
Os037973_01
Os037986_01
Os037996_01
Os037998_01
Os038027_01
Os038115_01
Os038137_01
Os038148_01
Os038157_01
Os038185_01
Os038267_01
Os038268_01
Os038331_01
Os038333_01
Os038340_01
Os038351_01
Os038400_01
Os038408_01
Os038453_01
Os038506_01
Os038522_01
Os038615_01
Os038671_01
Os038715_01
Os038808_01
Os038843_01
Os038884_01
Os038992_01
Os039222_01
Os039361_01
Os039391_01
Os039393_01
Os039430_01
Os039566_01
Os039701_01
Os039755_01
Os039757_01
Os039759_01
Os039769_01
Os039780_01
Os039790_01
Os039858_01
Os039892_01
Os040096_01
Os040182_01
Os040197_01
Os040209_01
Os040266_01
Os040272_01
Os040365_01
Os040420_01
Os040509_01
Os040565_01
Os040633_01
Os040695_01
Os040761_01
Os040784_01
Os040800_01
Os040805_01
Os040884_01
Os040954_01
Os040995_01
Os041019_01
Os041105_01
Os041196_01
Os041292_01
Os041297_01
Os041320_01
Os041364_01
Os041401_01
Os041436_01
Os041515_01
Os041539_01
Os041623_01
Os041666_01
Os041710_01
Os041752_01
Os041792_01
Os041962_01
Os041992_01
Os042060_01
Os042068_01
Os042253_01
Os042254_01
Os042332_01
Os042438_01
Os042450_01
Os042458_01
Os042717_01
Os042814_01
Os042815_01
Os042871_01
Os042932_01
Os042997_01
Os043108_01
Os043224_01
Os043227_01
Os043264_01
Os043323_01
Os043477_01
Os043506_01
Os043523_01
Os043894_01
Os044048_01
Os044218_01
Os044227_01
Os044260_01
Os044264_01
Os044296_01
Os044320_01
Os044323_01
Os044347_01
Os044363_01
Os044406_01
Os044412_01
Os044413_01
Os044430_01
Os044439_01
Os044441_01
Os044451_01
Os044455_01
Os044486_01
Os044541_01
Os044570_01
Os044590_01
Os044645_01
Os044656_01
Os044676_01
Os044699_01
Os044720_01
Os044733_01
Os044785_01
Os044815_01
Os044856_01
Os044859_01
Os044942_01
Os044972_01
Os045023_01
Os045036_01
Os045199_01
Os045320_01
Os045428_01
Os045483_01
Os045573_01
Os045606_01
Os045661_01
Os045670_01
Os045676_01
Os045716_01
Os045845_01
Os045846_01
Os045972_01
Os045999_01
Os046056_01
Os046103_01
Os046122_01
Os046132_01
Os046252_01
Os046476_01
Os046501_01
Os046505_01
Os046721_01
Os046822_01
Os047065_01
Os047118_01
Os047200_01
Os047235_01
Os047300_01
Os047308_01
Os047309_01
Os047323_01
Os047329_01
Os047350_01
Os047362_01
Os047363_01
Os047403_01
Os047414_01
Os047417_01
Os047418_01
Os047419_01
Os047444_01
Os047449_01
Os047456_01
Os047461_01
Os047481_01
Os047497_01
Os047505_01
Os047519_01
Os047523_01
Os047525_01
Os047539_01
Os047543_01
Os047554_01
Os047560_01
Os047587_01
Os047589_01
Os047611_01
Os047614_01
Os047657_01
Os047676_01
Os047754_01
Os047800_01
Os047914_01
Os047942_01
Os047953_01
Os047959_01
Os047981_01
Os048008_01
Os048165_01
Os048300_01
Os048332_01
Os048375_01
Os048615_01
Os048678_01
Os048795_01
Os048836_01
Os048930_01
Os048935_01
Os048985_01
Os049013_01
Os049037_01
Os049052_01
Os049139_01
Os049346_01
Os049381_01
Os049524_01
Os049608_01
Os049772_01
Os050109_01
Os050289_01
Os050323_01
Os050485_01
Os050498_01
Os050541_01
Os050558_01
Os050736_01
Os050864_01
Os050912_01
Os051035_01
Os051072_01
Os051120_01
Os051448_01
Os051507_01
Os051704_01
Os051865_01
Os051869_01
Os051959_01
Os051970_01
Os051973_01
Os051978_01
Os052001_01
Os052009_01
Os052015_01
Os052018_01
Os052036_01
Os052049_01
Os052050_01
Os052052_01
Os052060_01
Os052084_01
Os052088_01
Os052123_01
Os052184_01
Os052185_01
Os052206_01
Os052233_01
Os052271_01
Os052290_01
Os052297_01
Os052347_01
Os052350_01
Os052384_01
Os052414_01
Os052419_01
Os052424_01
Os052425_01
Os052449_01
Os052511_01
Os052580_01
Os052620_01
Os052629_01
Os052643_01
Os052653_01
Os052688_01
Os052709_01
Os052715_01
Os052727_01
Os052738_01
Os052745_01
Os052762_01
Os052766_01
Os052776_01
Os052791_01
Os052815_01
Os052828_01
Os052846_01
Os052872_01
Os052905_01
Os052919_01
Os052956_01
Os053032_01
Os053037_01
Os053119_01
Os053135_01
Os053166_01
Os053175_01
Os053281_01
Os053306_01
Os053328_01
Os053484_01
Os053512_01
Os053518_01
Os053523_01
Os053524_01
Os053557_01
Os053583_01
Os053595_01
Os053672_01
Os053720_01
Os053826_01
Os053840_01
Os053855_01
Os053883_01
Os053884_01
Os053940_01
Os053941_01
Os054032_01
Os054057_01
Os054072_01
Os054106_01
Os054140_02
Os054146_01
Os054166_01
Os054193_01
Os054202_01
Os054234_01
Os054238_01
Os054268_01
Os054273_01
Os054366_01
Os054377_01
Os054397_01
Os054406_01
Os054416_01
Os054430_01
Os054432_01
Os054447_01
Os054448_01
Os054452_01
Os054455_01
Os054459_01
Os054468_01
Os054489_01
Os054499_01
Os054508_01
Os054509_01
Os054516_01
Os054518_01
Os054539_01
Os054550_01
Os054579_01
Os054583_01
Os054588_01
Os054625_01
Os054627_01
Os054634_01
Os054653_01
Os054656_01
Os054681_01
Os054682_01
Os054726_01
Os054729_01
Os054752_01
Os054764_01
Os054768_01
Os054774_01
Os054785_01
Os054802_01
Os054826_01
Os054857_01
Os054918_01
Os054921_01
Os054947_01
Os054959_01
Os054979_01
Os054981_01
Os055002_01
Os055017_01
Os055029_01
Os055036_01
Os055085_01
Os055091_01
Os055098_01
Os055106_01
Os055113_01
Os055126_01
Os055169_02
Os055172_01
Os055174_01
Os055224_01
Os055241_01
Os055250_01
Os055292_01
Os055351_01
Os055361_01
Os055387_01
Os055406_01
Os055428_02
Os055460_01
Os055674_01
Os055790_01
Os055860_01
Os055966_01
Os055969_01
Os056024_01
Os056074_02
Os056106_01
Os056219_01
Os056222_01
Os056237_01
Os056288_01
Os056321_01
Os056322_01
Os056330_01
Os056334_01
Os056340_01
Os056342_01
Os056354_01
Os056387_01
Os056388_01
Os056473_01
Os056488_01
Os056492_01
Os056528_01
Os056531_01
Os056544_01
Os056562_01
Os056574_01
Os056589_01
Os056622_02
Os056651_01
Os056653_01
Os056660_01
Os056663_01
Os056700_01
Os056702_01
Os056714_01
Os056722_01
Os056739_01
Os056746_01
Os056754_01
Os056775_01
Os056780_01
Os056782_01
Os056785_01
Os056788_01
Os056800_01
Os056809_01
Os056835_01
Os056838_01
Os056844_01
Os056851_01
Os056866_01
Os056872_01
Os056936_01
Os056971_02
Os056975_02
Os056989_02
Os057029_01
Os057032_02
Os057094_01
Os057106_02
Os057133_02
Os057155_02
Os057159_01
Os057179_02
Os057258_02
Os057297_02
Os057327_02
Os057331_01
Os057411_02
Os057451_02
Os057454_02
Os057570_02
Os057666_02

SUPPLEMENTAL TABLE 7
Oligo ID
Os000014_01
Os000030_01
Os000035_02
Os000044_01
Os000158_01
Os000174_01
Os000185_01
Os000205_01
Os000241_01
Os000312_01
Os000314_01
Os000333_01
Os000340_01
Os000393_01
Os000403_01
Os000428_01
Os000429_01
Os000434_01
Os000437_01
Os000508_01
Os000513_01
Os000517_01
Os000532_01
Os000545_01
Os000568_01
Os000597_01
Os000627_01
Os000664_01
Os000669_01
Os000691_01
Os000698_01
Os000719_01
Os000720_01
Os000757_01
Os000761_01
Os000786_02
Os000836_01
Os000842_01
Os000862_01
Os000893_01
Os000928_01
Os000942_01
Os000969_01
Os000997_01
Os001035_01
Os001046_01
Os001073_01
Os001087_01
Os001107_01
Os001127_01
Os001225_01
Os001234_01
Os001238_01
Os001260_01
Os001277_01
Os001290_01
Os001296_01
Os001300_02
Os001320_01
Os001325_01
Os001377_01
Os001461_01
Os001497_01
Os001513_01
Os001529_01
Os001537_01
Os001555_01
Os001565_01
Os001620_01
Os001621_01
Os001631_01
Os001649_01
Os001666_01
Os001667_01
Os001778_01
Os001806_01
Os001842_01
Os001847_01
Os001870_01
Os001900_01
Os001903_01
Os001920_01
Os001925_01
Os001930_01
Os001937_01
Os001941_01
Os001972_01
Os002008_01
Os002028_01
Os002092_01
Os002138_01
Os002150_01
Os002160_01
Os002166_02
Os002390_01
Os002532_01
Os002546_01
Os002550_01
Os002652_01
Os002696_01
Os002732_01
Os002746_01
Os002753_01
Os002903_01
Os003026_01
Os003284_01
Os003312_01
Os003326_01
Os003332_01
Os003443_01
Os003485_01
Os003719_01
Os003776_01
Os003800_01
Os003986_01
Os004298_01
Os004301_01
Os004343_01
Os004656_01
Os004740_01
Os004794_01
Os004928_01
Os005642_01
Os006042_02
Os006070_01
Os006108_01
Os006335_02
Os006546_02
Os006678_01
Os007068_01
Os007171_01
Os007193_02
Os007195_01
Os007671_01
Os007764_01
Os007857_01
Os007891_01
Os007912_02
Os007913_02
Os007952_01
Os008196_01
Os008273_01
Os008320_01
Os008374_01
Os008400_01
Os008475_01
Os008485_01
Os008487_01
Os008495_01
Os008500_01
Os008538_01
Os008546_01
Os008560_01
Os008562_01
Os008565_01
Os008571_01
Os008595_01
Os008596_01
Os008602_01
Os008615_01
Os008624_01
Os008626_01
Os008629_01
Os008635_01
Os008639_01
Os008643_01
Os008648_01
Os008675_01
Os008728_01
Os008729_01
Os008732_01
Os008742_01
Os008761_01
Os008762_01
Os008773_01
Os008775_01
Os008777_01
Os008795_01
Os008803_01
Os008837_01
Os008839_01
Os008842_01
Os008886_01
Os008889_01
Os008973_01
Os009011_01
Os009016_01
Os009032_01
Os009039_01
Os009047_01
Os009063_01
Os009101_01
Os009115_01
Os009117_01
Os009161_01
Os009164_01
Os009199_01
Os009211_01
Os009229_01
Os009242_01
Os009246_01
Os009261_01
Os009268_01
Os009290_01
Os009298_01
Os009303_01
Os009338_01
Os009351_01
Os009353_01
Os009357_01
Os009362_01
Os009365_01
Os009373_01
Os009390_01
Os009393_01
Os009425_01
Os009433_01
Os009459_01
Os009529_01
Os009534_01
Os009556_01
Os009602_01
Os009619_01
Os009637_01
Os009679_01
Os009682_01
Os009694_01
Os009705_01
Os009744_01
Os009764_01
Os009800_01
Os009805_01
Os009843_01
Os009858_01
Os009868_01
Os009895_01
Os009913_01
Os009927_01
Os009930_01
Os009971_01
Os009986_01
Os009989_01
Os010010_01
Os010064_01
Os010113_01
Os010117_01
Os010122_01
Os010145_01
Os010160_01
Os010171_01
Os010206_01
Os010219_01
Os010239_01
Os010272_01
Os010278_01
Os010317_01
Os010322_01
Os010372_01
Os010407_01
Os010480_01
Os010486_01
Os010501_01
Os010554_01
Os010560_01
Os010568_01
Os010599_01
Os010606_01
Os010610_01
Os010621_01
Os010643_01
Os010685_01
Os010710_01
Os010713_01
Os010721_01
Os010780_01
Os010789_01
Os010804_01
Os010807_01
Os010809_01
Os010835_01
Os010861_01
Os010863_01
Os010867_01
Os010880_01
Os010883_01
Os010894_01
Os010903_01
Os010927_01
Os010941_01
Os010959_01
Os010968_01
Os010976_01
Os010985_01
Os011001_01
Os011016_01
Os011018_01
Os011031_01
Os011080_01
Os011103_01
Os011105_01
Os011113_01
Os011216_01
Os011224_01
Os011225_01
Os011231_01
Os011241_01
Os011247_01
Os011249_01
Os011252_01
Os011257_01
Os011262_01
Os011265_01
Os011311_01
Os011323_01
Os011327_01
Os011350_01
Os011352_01
Os011359_01
Os011360_01
Os011393_01
Os011414_01
Os011435_01
Os011437_01
Os011452_01
Os011522_01
Os011563_01
Os011574_01
Os011610_01
Os011637_01
Os011653_01
Os011670_01
Os011673_01
Os011685_01
Os011692_01
Os011700_01
Os011707_01
Os011762_01
Os011785_01
Os011787_01
Os011818_01
Os011838_01
Os011851_01
Os011870_01
Os011875_01
Os011919_01
Os011937_01
Os011959_01
Os011961_01
Os011972_01
Os011994_01
Os012021_01
Os012023_01
Os012035_01
Os012042_01
Os012049_01
Os012066_01
Os012089_01
Os012114_01
Os012153_01
Os012157_01
Os012160_01
Os012175_01
Os012193_01
Os012232_01
Os012237_01
Os012245_01
Os012259_01
Os012278_01
Os012286_01
Os012287_01
Os012292_01
Os012330_01
Os012363_01
Os012366_01
Os012383_01
Os012402_01
Os012405_01
Os012413_01
Os012419_01
Os012424_01
Os012444_01
Os012472_01
Os012479_01
Os012497_01
Os012503_01
Os012505_01
Os012511_01
Os012520_01
Os012538_01
Os012543_01
Os012553_01
Os012556_01
Os012586_01
Os012588_01
Os012603_01
Os012619_01
Os012623_01
Os012632_01
Os012640_01
Os012645_01
Os012660_01
Os012716_01
Os012731_01
Os012738_01
Os012777_01
Os012785_01
Os012807_01
Os012813_01
Os012816_01
Os012821_01
Os012865_01
Os012900_01
Os012902_01
Os012925_01
Os012927_01
Os012946_01
Os012947_01
Os012955_01
Os012960_01
Os012984_01
Os013023_01
Os013030_01
Os013036_01
Os013042_01
Os013103_01
Os013108_01
Os013114_01
Os013119_01
Os013125_01
Os013128_01
Os013153_01
Os013158_01
Os013169_01
Os013175_01
Os013186_01
Os013189_01
Os013191_01
Os013192_01
Os013239_01
Os013246_01
Os013325_01
Os013346_01
Os013388_01
Os013432_01
Os013441_01
Os013447_01
Os013462_01
Os013468_01
Os013473_01
Os013504_01
Os013536_01
Os013582_01
Os013598_01
Os013613_01
Os013617_01
Os013634_01
Os013654_01
Os013659_01
Os013676_01
Os013721_01
Os013749_01
Os013762_01
Os013810_01
Os013812_01
Os013817_01
Os013838_01
Os013841_01
Os013844_01
Os013847_01
Os013849_01
Os013854_01
Os013857_01
Os013863_01
Os013867_01
Os013877_01
Os013885_01
Os013934_01
Os013985_01
Os013986_01
Os014026_01
Os014027_01
Os014040_01
Os014055_01
Os014062_01
Os014063_01
Os014092_01
Os014127_01
Os014129_01
Os014133_01
Os014154_01
Os014157_01
Os014197_01
Os014216_01
Os014245_01
Os014284_01
Os014316_01
Os014317_01
Os014347_01
Os014430_01
Os014438_01
Os014441_01
Os014442_01
Os014467_01
Os014505_01
Os014512_01
Os014533_01
Os014539_01
Os014585_01
Os014590_01
Os014604_01
Os014631_01
Os014634_01
Os014654_01
Os014662_01
Os014664_01
Os014673_01
Os014706_01
Os014707_01
Os014717_01
Os014735_01
Os014747_01
Os014761_01
Os014766_01
Os014773_01
Os014801_01
Os014803_01
Os014815_01
Os014835_01
Os014836_01
Os014839_01
Os014844_01
Os014876_01
Os014892_01
Os014909_01
Os014927_01
Os014944_01
Os014962_01
Os014967_01
Os014969_01
Os014990_01
Os015027_01
Os015040_01
Os015043_01
Os015073_01
Os015085_01
Os015136_01
Os015148_01
Os015149_01
Os015152_01
Os015156_01
Os015303_01
Os015317_01
Os015470_01
Os015554_01
Os015556_01
Os015581_01
Os015594_01
Os015599_01
Os015601_01
Os015610_01
Os015617_01
Os015621_01
Os015623_01
Os015629_01
Os015635_01
Os015651_01
Os015656_01
Os015661_01
Os015684_01
Os015738_01
Os015790_01
Os015844_01
Os015849_01
Os015860_01
Os015866_01
Os015868_01
Os015880_01
Os015902_01
Os015910_01
Os015916_01
Os015961_01
Os015985_01
Os015991_01
Os015995_01
Os015997_01
Os015998_01
Os016013_01
Os016029_01
Os016030_01
Os016064_01
Os016068_01
Os016071_01
Os016074_01
Os016102_01
Os016128_01
Os016141_01
Os016165_01
Os016187_01
Os016203_01
Os016204_01
Os016205_01
Os016215_01
Os016220_01
Os016225_01
Os016227_01
Os016232_01
Os016243_01
Os016299_01
Os016300_01
Os016305_01
Os016309_01
Os016373_01
Os016382_01
Os016406_01
Os016423_01
Os016428_01
Os016432_01
Os016458_01
Os016461_01
Os016483_01
Os016486_01
Os016505_01
Os016515_01
Os016523_01
Os016536_01
Os016540_01
Os016556_01
Os016593_01
Os016601_01
Os016614_01
Os016652_01
Os016668_01
Os016676_01
Os016693_01
Os016704_01
Os016747_01
Os016757_01
Os016768_01
Os016780_01
Os016793_01
Os016798_01
Os016813_01
Os016826_01
Os016837_01
Os016871_01
Os016888_01
Os016901_01
Os016903_01
Os016919_01
Os016925_01
Os016933_01
Os016940_01
Os016969_01
Os016979_01
Os017003_01
Os017037_01
Os017040_01
Os017053_01
Os017058_01
Os017111_01
Os017129_01
Os017156_01
Os017161_01
Os017163_01
Os017185_01
Os017217_01
Os017225_01
Os017243_01
Os017245_01
Os017255_01
Os017259_01
Os017274_01
Os017279_01
Os017289_01
Os017306_01
Os017328_01
Os017336_01
Os017341_01
Os017364_01
Os017367_01
Os017376_01
Os017396_01
Os017407_01
Os017443_01
Os017457_01
Os017475_01
Os017489_01
Os017526_01
Os017544_01
Os017577_01
Os017603_01
Os017620_01
Os017624_01
Os017656_01
Os017664_01
Os017680_01
Os017686_01
Os017696_01
Os017701_01
Os017707_01
Os017710_01
Os017726_01
Os017764_01
Os017806_01
Os017831_01
Os017833_01
Os017879_01
Os017881_01
Os017890_01
Os017893_01
Os017907_01
Os017931_01
Os017954_01
Os017966_01
Os018034_01
Os018042_01
Os018044_01
Os018046_01
Os018049_01
Os018054_01
Os018065_01
Os018078_01
Os018085_01
Os018087_01
Os018092_01
Os018110_01
Os018111_01
Os018112_01
Os018115_01
Os018122_01
Os018134_01
Os018144_01
Os018147_01
Os018148_01
Os018173_01
Os018188_01
Os018190_01
Os018198_01
Os018211_01
Os018253_01
Os018255_01
Os018279_01
Os018311_01
Os018314_01
Os018326_01
Os018327_01
Os018334_01
Os018337_01
Os018341_01
Os018362_01
Os018368_01
Os018381_01
Os018393_01
Os018409_01
Os018470_01
Os018478_01
Os018487_01
Os018501_01
Os018509_01
Os018608_01
Os018626_01
Os018666_01
Os018673_01
Os018690_01
Os018696_01
Os018699_01
Os018713_01
Os018725_01
Os018730_01
Os018734_01
Os018737_01
Os018739_01
Os018757_01
Os018767_01
Os018776_01
Os018786_01
Os018795_01
Os018818_01
Os018841_01
Os018863_01
Os018880_01
Os018900_01
Os018909_01
Os018916_01
Os018940_01
Os018953_01
Os018961_01
Os018965_01
Os018981_01
Os018989_01
Os019008_01
Os019020_01
Os019022_01
Os019036_01
Os019045_01
Os019066_01
Os019095_01
Os019099_01
Os019114_01
Os019124_01
Os019159_01
Os019191_01
Os019192_01
Os019193_01
Os019194_01
Os019196_01
Os019234_01
Os019270_01
Os019279_01
Os019289_01
Os019296_01
Os019313_01
Os019343_01
Os019350_01
Os019364_01
Os019405_01
Os019439_01
Os019452_01
Os019483_01
Os019495_01
Os019512_01
Os019516_01
Os019519_01
Os019536_01
Os019547_01
Os019578_01
Os019622_01
Os019627_01
Os019629_01
Os019652_01
Os019656_01
Os019662_01
Os019663_01
Os019664_01
Os019701_01
Os019716_01
Os019749_01
Os019756_01
Os019792_01
Os019804_01
Os019809_01
Os019824_01
Os019825_01
Os019832_01
Os019855_01
Os019891_01
Os019896_01
Os019901_01
Os019927_01
Os019929_01
Os019941_01
Os019943_01
Os019957_01
Os019974_01
Os019977_01
Os020008_01
Os020016_01
Os020035_01
Os020042_01
Os020073_01
Os020117_01
Os020119_01
Os020130_01
Os020145_01
Os020194_01
Os020216_01
Os020217_01
Os020228_01
Os020230_01
Os020252_01
Os020261_01
Os020269_01
Os020321_01
Os020360_01
Os020374_01
Os020397_01
Os020411_01
Os020412_01
Os020424_01
Os020426_01
Os020450_01
Os020546_01
Os020551_01
Os020564_01
Os020584_01
Os020597_01
Os020624_01
Os020632_01
Os020633_01
Os020653_01
Os020661_01
Os020694_01
Os020703_01
Os020707_01
Os020731_01
Os020749_01
Os020769_01
Os020796_01
Os020797_01
Os020838_01
Os020841_01
Os020842_01
Os020847_01
Os020903_01
Os020906_01
Os020908_01
Os020909_01
Os020923_01
Os020925_01
Os020930_01
Os020934_01
Os020935_01
Os020936_01
Os020942_01
Os020945_01
Os020961_01
Os020973_01
Os021006_01
Os021018_01
Os021051_01
Os021095_01
Os021156_01
Os021165_01
Os021173_01
Os021198_01
Os021199_01
Os021202_01
Os021231_01
Os021254_01
Os021270_01
Os021287_01
Os021313_01
Os021357_01
Os021372_01
Os021381_01
Os021382_01
Os021429_01
Os021443_01
Os021445_01
Os021455_01
Os021471_01
Os021488_01
Os021509_01
Os021511_01
Os021518_01
Os021523_01
Os021533_01
Os021547_01
Os021555_01
Os021597_01
Os021603_01
Os021632_01
Os021642_01
Os021643_01
Os021726_01
Os021735_01
Os021768_01
Os021770_01
Os021844_01
Os021845_01
Os021846_01
Os021857_01
Os021880_01
Os021891_01
Os021907_01
Os021912_01
Os021942_01
Os021946_01
Os021954_01
Os021987_01
Os021989_01
Os021996_01
Os022021_01
Os022024_01
Os022028_01
Os022036_01
Os022043_01
Os022078_01
Os022087_01
Os022116_01
Os022133_01
Os022161_01
Os022167_01
Os022207_01
Os022209_01
Os022239_01
Os022265_01
Os022272_01
Os022279_01
Os022341_01
Os022373_01
Os022389_01
Os022404_01
Os022409_01
Os022416_01
Os022420_01
Os022421_01
Os022431_01
Os022433_01
Os022448_01
Os022477_01
Os022513_01
Os022519_01
Os022579_01
Os022584_01
Os022597_01
Os022684_01
Os022691_01
Os022692_01
Os022756_01
Os022790_01
Os022837_01
Os022844_01
Os022866_01
Os022876_01
Os022883_01
Os022919_01
Os022927_01
Os022955_01
Os022969_01
Os022976_01
Os022994_01
Os023011_01
Os023018_01
Os023044_01
Os023056_01
Os023060_01
Os023063_01
Os023116_01
Os023117_01
Os023165_01
Os023350_01
Os023465_01
Os023489_01
Os023510_01
Os023542_01
Os023546_01
Os023552_01
Os023582_01
Os023596_01
Os023597_01
Os023620_01
Os023625_01
Os023644_01
Os023671_01
Os023695_01
Os023696_01
Os023706_01
Os023709_01
Os023766_01
Os023774_01
Os023781_01
Os023810_01
Os023848_01
Os023849_01
Os023851_01
Os023903_01
Os023925_01
Os023931_01
Os023964_01
Os024014_01
Os024024_01
Os024032_01
Os024037_01
Os024044_01
Os024079_01
Os024083_01
Os024094_01
Os024218_01
Os024220_01
Os024232_01
Os024233_01
Os024260_01
Os024271_01
Os024305_01
Os024309_01
Os024415_01
Os024445_01
Os024474_01
Os024514_01
Os024541_01
Os024549_01
Os024550_01
Os024561_01
Os024563_01
Os024577_01
Os024580_01
Os024621_01
Os024652_01
Os024751_01
Os024763_01
Os024773_01
Os024808_01
Os024824_01
Os024836_01
Os024855_01
Os024863_01
Os024934_01
Os024963_01
Os025036_01
Os025051_01
Os025063_01
Os025078_01
Os025225_01
Os025626_01
Os025638_01
Os025683_01
Os025691_01
Os025804_01
Os025848_01
Os025886_01
Os025899_01
Os025997_01
Os026012_01
Os026021_01
Os026060_01
Os026070_01
Os026143_01
Os026168_01
Os026264_01
Os026287_01
Os026333_01
Os026341_01
Os026349_01
Os026434_01
Os026674_01
Os026758_01
Os027325_01
Os027338_01
Os027591_01
Os027606_01
Os027608_01
Os027642_01
Os027650_01
Os027819_01
Os027869_01
Os027971_01
Os028061_01
Os028119_01
Os028127_01
Os028182_01
Os028416_01
Os028481_01
Os028491_01
Os028586_01
Os028594_01
Os028676_01
Os028693_01
Os028712_01
Os028746_01
Os028913_01
Os028973_01
Os029007_01
Os029023_01
Os029024_01
Os029025_01
Os029069_01
Os029226_01
Os029370_01
Os029395_01
Os029446_01
Os029617_01
Os029620_01
Os029670_01
Os029689_01
Os029761_01
Os029783_01
Os029826_01
Os029833_01
Os029853_01
Os030002_01
Os030007_01
Os030046_01
Os030071_01
Os030101_01
Os030104_01
Os030128_01
Os030139_01
Os030170_01
Os030206_01
Os030236_01
Os030297_01
Os030331_01
Os030343_01
Os030354_01
Os030422_01
Os030451_01
Os030504_01
Os030550_01
Os030616_01
Os030625_01
Os030757_01
Os030792_01
Os030802_01
Os030861_01
Os030919_01
Os030928_01
Os030984_01
Os030996_01
Os031071_01
Os031094_01
Os031145_01
Os031186_01
Os031218_01
Os031364_01
Os031466_01
Os031468_01
Os031525_01
Os031559_01
Os031591_01
Os031609_01
Os031712_01
Os031737_01
Os031896_01
Os031912_01
Os031927_01
Os031989_01
Os032009_01
Os032027_01
Os032029_01
Os032115_01
Os032244_01
Os032293_01
Os032388_01
Os032429_01
Os032435_01
Os032446_01
Os032448_01
Os032486_01
Os032547_01
Os032597_01
Os032710_01
Os032815_01
Os032862_01
Os032871_01
Os032911_01
Os032940_01
Os032968_01
Os032997_01
Os033011_01
Os033022_01
Os033043_01
Os033096_01
Os033133_01
Os033211_01
Os033293_01
Os033351_01
Os033376_01
Os033451_01
Os033459_01
Os033700_01
Os033835_01
Os033901_01
Os033909_01
Os033917_01
Os033986_01
Os034076_01
Os034084_01
Os034222_01
Os034238_01
Os034270_01
Os034315_01
Os034581_01
Os034833_01
Os034877_01
Os034884_01
Os034943_01
Os035032_01
Os035258_01
Os035283_01
Os035447_01
Os035599_01
Os035629_01
Os035852_01
Os035905_01
Os035911_01
Os036080_01
Os036093_01
Os036161_01
Os036172_01
Os036256_01
Os036286_01
Os036292_01
Os036310_01
Os036380_01
Os036381_01
Os036397_01
Os036569_01
Os036791_01
Os036839_01
Os036962_01
Os036980_01
Os036995_01
Os037029_01
Os037055_01
Os037056_01
Os037254_01
Os037264_01
Os037460_01
Os037697_01
Os037726_01
Os037758_01
Os037804_01
Os037825_01
Os038067_01
Os038147_01
Os038174_01
Os038195_01
Os038407_01
Os038428_01
Os038577_01
Os038651_01
Os038694_01
Os038901_01
Os038926_01
Os039270_01
Os039368_01
Os039501_01
Os039505_01
Os039547_01
Os039556_01
Os039703_01
Os039791_01
Os040009_01
Os040051_01
Os040123_01
Os040220_01
Os040381_01
Os040584_01
Os040600_01
Os040624_01
Os040902_01
Os040908_01
Os040957_01
Os041003_01
Os041166_01
Os041325_01
Os041523_01
Os041571_01
Os041643_01
Os041754_01
Os041918_01
Os041919_01
Os041931_01
Os042042_01
Os042047_01
Os042357_01
Os042707_01
Os042953_01
Os043037_01
Os043090_01
Os043153_01
Os043206_01
Os043473_01
Os043506_01
Os043649_01
Os043723_01
Os043803_01
Os043866_01
Os043897_01
Os044093_01
Os044197_01
Os044290_01
Os044610_01
Os044770_01
Os044890_01
Os044998_01
Os045194_01
Os045466_01
Os045562_01
Os045618_01
Os045654_01
Os045723_01
Os045964_01
Os046076_01
Os046174_01
Os046315_01
Os046324_01
Os046382_01
Os046515_01
Os046769_01
Os046888_01
Os046889_01
Os047164_01
Os047671_01
Os047738_01
Os047819_01
Os048254_01
Os048698_01
Os048923_01
Os049226_01
Os049262_01
Os049451_01
Os049899_01
Os050111_01
Os050182_01
Os050351_01
Os050627_01
Os050741_01
Os050799_01
Os051482_01
Os051804_01
Os051856_01
Os051919_01
Os051935_01
Os051937_01
Os051938_01
Os051953_01
Os051965_01
Os051966_01
Os051971_01
Os051974_01
Os051980_01
Os051981_01
Os051997_01
Os052000_01
Os052001_01
Os052012_01
Os052035_01
Os052071_01
Os052074_01
Os052094_01
Os052096_01
Os052099_01
Os052101_01
Os052105_01
Os052111_01
Os052148_01
Os052152_01
Os052175_01
Os052176_01
Os052198_01
Os052203_01
Os052215_01
Os052221_01
Os052225_01
Os052230_01
Os052231_01
Os052236_01
Os052239_01
Os052240_01
Os052251_01
Os052270_01
Os052288_01
Os052311_01
Os052316_01
Os052331_01
Os052337_01
Os052361_01
Os052370_01
Os052384_01
Os052387_01
Os052413_01
Os052424_01
Os052436_01
Os052464_01
Os052466_01
Os052482_01
Os052507_01
Os052515_01
Os052531_01
Os052557_01
Os052562_01
Os052580_01
Os052593_01
Os052594_01
Os052597_01
Os052654_01
Os052658_01
Os052668_01
Os052669_01
Os052672_01
Os052675_01
Os052686_01
Os052717_01
Os052745_01
Os052753_01
Os052767_01
Os052782_02
Os052809_01
Os052819_01
Os052826_01
Os052844_01
Os052878_01
Os052888_01
Os052913_01
Os052939_01
Os053000_01
Os053018_01
Os053043_01
Os053047_01
Os053057_01
Os053136_01
Os053180_01
Os053206_01
Os053208_01
Os053249_01
Os053316_01
Os053431_01
Os053503_01
Os053510_01
Os053731_02
Os053758_01
Os053922_01
Os053942_01
Os053954_01
Os053990_01
Os054013_01
Os054035_01
Os054086_02
Os054260_01
Os054272_01
Os054316_01
Os054377_01
Os054381_01
Os054395_01
Os054424_01
Os054428_01
Os054431_01
Os054469_01
Os054493_01
Os054495_01
Os054496_01
Os054507_01
Os054542_01
Os054543_01
Os054553_01
Os054556_01
Os054558_01
Os054562_01
Os054564_01
Os054568_01
Os054569_01
Os054586_01
Os054590_01
Os054595_01
Os054599_01
Os054606_01
Os054616_01
Os054621_01
Os054626_01
Os054634_01
Os054637_01
Os054639_01
Os054643_01
Os054652_01
Os054659_01
Os054676_01
Os054678_01
Os054680_01
Os054681_01
Os054682_01
Os054684_01
Os054685_01
Os054687_01
Os054698_01
Os054699_01
Os054700_01
Os054701_01
Os054724_01
Os054727_01
Os054728_01
Os054733_01
Os054737_01
Os054743_01
Os054753_01
Os054767_01
Os054770_01
Os054789_01
Os054790_01
Os054804_01
Os054808_01
Os054809_01
Os054817_01
Os054824_01
Os054828_01
Os054830_01
Os054835_01
Os054867_01
Os054880_01
Os054882_01
Os054885_01
Os054889_01
Os054898_01
Os054900_01
Os054901_01
Os054908_01
Os054909_01
Os054915_01
Os054922_01
Os054925_01
Os054934_01
Os054974_01
Os054977_01
Os054983_01
Os054996_01
Os055018_01
Os055028_01
Os055061_01
Os055071_01
Os055079_01
Os055094_01
Os055103_01
Os055136_01
Os055156_01
Os055161_01
Os055190_01
Os055205_01
Os055267_01
Os055296_01
Os055299_01
Os055308_01
Os055319_01
Os055359_01
Os055408_01
Os055410_01
Os055414_01
Os055421_01
Os055427_01
Os055430_01
Os055458_01
Os055470_01
Os055473_01
Os055497_01
Os055504_01
Os055510_01
Os055624_01
Os055713_01
Os055830_01
Os055961_01
Os055962_01
Os056019_01
Os056035_01
Os056045_02
Os056123_01
Os056145_01
Os056162_01
Os056174_01
Os056193_01
Os056209_01
Os056250_01
Os056260_01
Os056352_02
Os056390_01
Os056424_01
Os056606_01
Os056712_01
Os056731_01
Os056821_01
Os056921_02
Os056933_02
Os056940_02
Os056946_01
Os056979_02
Os056980_02
Os056998_02
Os057014_02
Os057016_02
Os057021_02
Os057045_02
Os057063_02
Os057133_02
Os057175_02
Os057245_02
Os057253_02
Os057285_02
Os057304_02
Os057327_02
Os057394_02
Os057402_02
Os057410_02
Os057433_02
Os057435_02
Os057491_02
Os057614_02
Os057659_02

SUPPLEMENTAL TABLE 8
Oligo ID
Os000043_01
Os000062_01
Os000073_01
Os000085_01
Os000111_01
Os000150_01
Os000151_01
Os000152_01
Os000160_01
Os000165_01
Os000208_01
Os000237_01
Os000262_01
Os000271_01
Os000289_01
Os000322_01
Os000366_01
Os000367_01
Os000380_01
Os000386_01
Os000443_01
Os000548_01
Os000629_01
Os000842_01
Os000957_01
Os001220_01
Os001284_01
Os001330_01
Os001385_01
Os001458_01
Os001472_01
Os001499_01
Os001637_01
Os001770_01
Os001810_01
Os001921_01
Os001957_01
Os001958_01
Os001984_01
Os001993_01
Os002020_01
Os002181_01
Os002395_01
Os002422_01
Os002476_01
Os002500_01
Os002534_01
Os002537_01
Os002599_01
Os002648_01
Os002821_01
Os002918_01
Os003091_01
Os003691_01
Os003765_01
Os004097_01
Os004148_01
Os004265_01
Os004535_01
Os004563_02
Os004746_02
Os004761_01
Os005179_01
Os005779_01
Os005850_01
Os006904_01
Os007202_01
Os007263_01
Os007271_01
Os007638_01
Os007780_01
Os007814_01
Os007861_01
Os007867_01
Os007873_01
Os007907_01
Os007934_01
Os008046_01
Os008101_01
Os008105_01
Os008133_01
Os008158_01
Os008213_01
Os008225_01
Os008286_01
Os008364_01
Os008414_01
Os008420_01
Os008423_01
Os008553_01
Os008564_01
Os008569_01
Os008589_01
Os008612_01
Os008618_01
Os008620_01
Os008640_01
Os008657_01
Os008668_01
Os008682_01
Os008685_01
Os008693_01
Os008712_01
Os008796_01
Os008859_01
Os008866_01
Os008869_01
Os008878_01
Os008888_01
Os008900_01
Os008904_01
Os008923_01
Os008955_01
Os008967_01
Os008979_01
Os009025_01
Os009043_01
Os009081_01
Os009095_01
Os009138_01
Os009150_01
Os009156_01
Os009234_01
Os009266_01
Os009272_01
Os009280_01
Os009296_01
Os009308_01
Os009346_01
Os009348_01
Os009448_01
Os009478_01
Os009484_01
Os009490_01
Os009499_01
Os009506_01
Os009509_01
Os009518_01
Os009532_01
Os009545_01
Os009547_01
Os009558_01
Os009577_01
Os009586_01
Os009599_01
Os009604_01
Os009638_01
Os009639_01
Os009657_01
Os009664_01
Os009671_01
Os009711_01
Os009724_01
Os009785_01
Os009786_01
Os009844_01
Os009870_01
Os009877_01
Os009883_01
Os009887_01
Os009906_01
Os009932_01
Os009969_01
Os009998_01
Os010018_01
Os010056_01
Os010076_01
Os010108_01
Os010109_01
Os010115_01
Os010175_01
Os010179_01
Os010197_01
Os010210_01
Os010294_01
Os010356_01
Os010375_01
Os010413_01
Os010414_01
Os010492_01
Os010517_01
Os010702_01
Os010727_01
Os010729_01
Os010763_01
Os010773_01
Os010779_01
Os010808_01
Os010812_01
Os010855_01
Os010900_01
Os010948_01
Os010954_01
Os010965_01
Os011082_01
Os011142_01
Os011176_01
Os011201_01
Os011232_01
Os011276_01
Os011278_01
Os011314_01
Os011349_01
Os011356_01
Os011389_01
Os011401_01
Os011511_01
Os011561_01
Os011580_01
Os011584_01
Os011592_01
Os011601_01
Os011603_01
Os011622_01
Os011626_01
Os011633_01
Os011644_01
Os011647_01
Os011655_01
Os011662_01
Os011669_01
Os011720_01
Os011723_01
Os011726_01
Os011735_01
Os011740_01
Os011748_01
Os011752_01
Os011760_01
Os011778_01
Os011784_01
Os011824_01
Os011833_01
Os011862_01
Os011871_01
Os011874_01
Os011879_01
Os011885_01
Os011886_01
Os011910_01
Os011925_01
Os011967_01
Os011970_01
Os011976_01
Os011977_01
Os012013_01
Os012014_01
Os012036_01
Os012069_01
Os012136_01
Os012151_01
Os012162_01
Os012166_01
Os012181_01
Os012189_01
Os012191_01
Os012213_01
Os012220_01
Os012235_01
Os012243_01
Os012251_01
Os012253_01
Os012305_01
Os012308_01
Os012360_01
Os012377_01
Os012379_01
Os012388_01
Os012399_01
Os012409_01
Os012417_01
Os012427_01
Os012464_01
Os012466_01
Os012477_01
Os012487_01
Os012508_01
Os012514_01
Os012532_01
Os012558_01
Os012583_01
Os012585_01
Os012617_01
Os012654_01
Os012662_01
Os012664_01
Os012666_01
Os012683_01
Os012686_01
Os012694_01
Os012753_01
Os012754_01
Os012765_01
Os012775_01
Os012811_01
Os012819_01
Os012829_01
Os012833_01
Os012844_01
Os012851_01
Os012866_01
Os012868_01
Os012905_01
Os012910_01
Os012921_01
Os012955_01
Os012966_01
Os013014_01
Os013020_01
Os013022_01
Os013025_01
Os013034_01
Os013062_01
Os013147_01
Os013150_01
Os013161_01
Os013166_01
Os013184_01
Os013185_01
Os013205_01
Os013207_01
Os013218_01
Os013226_01
Os013271_01
Os013283_01
Os013295_01
Os013300_01
Os013316_01
Os013320_01
Os013335_01
Os013340_01
Os013348_01
Os013389_01
Os013393_01
Os013398_01
Os013414_01
Os013425_01
Os013442_01
Os013443_01
Os013449_01
Os013465_01
Os013502_01
Os013509_01
Os013527_01
Os013541_01
Os013545_01
Os013546_01
Os013569_01
Os013589_01
Os013616_01
Os013645_01
Os013649_01
Os013650_01
Os013653_01
Os013712_01
Os013745_01
Os013799_01
Os013826_01
Os013845_01
Os013866_01
Os013895_01
Os013936_01
Os013945_01
Os013946_01
Os013952_01
Os013976_01
Os013979_01
Os013987_01
Os013991_01
Os014014_01
Os014037_01
Os014039_01
Os014048_01
Os014057_01
Os014071_01
Os014076_01
Os014081_01
Os014085_01
Os014102_01
Os014116_01
Os014146_01
Os014169_01
Os014189_01
Os014203_01
Os014210_01
Os014217_01
Os014222_01
Os014249_01
Os014252_01
Os014262_01
Os014263_01
Os014269_01
Os014281_01
Os014285_01
Os014292_01
Os014296_01
Os014315_01
Os014351_01
Os014364_01
Os014366_01
Os014373_01
Os014410_01
Os014423_01
Os014453_01
Os014454_01
Os014461_01
Os014470_01
Os014488_01
Os014499_01
Os014516_01
Os014522_01
Os014548_01
Os014552_01
Os014564_01
Os014568_01
Os014617_01
Os014648_01
Os014703_01
Os014740_01
Os014762_01
Os014776_01
Os014822_01
Os014849_01
Os014984_01
Os014994_01
Os015014_01
Os015041_01
Os015087_01
Os015125_01
Os015133_01
Os015282_01
Os015418_01
Os015534_01
Os015547_01
Os015552_01
Os015572_01
Os015593_01
Os015623_01
Os015626_01
Os015628_01
Os015644_01
Os015666_01
Os015678_01
Os015702_01
Os015706_01
Os015739_01
Os015797_01
Os015816_01
Os015820_01
Os015832_01
Os015835_01
Os015850_01
Os015872_01
Os015879_01
Os015881_01
Os015882_01
Os015896_01
Os015905_01
Os015909_01
Os015914_01
Os015935_01
Os015984_01
Os015996_01
Os016045_01
Os016057_01
Os016070_01
Os016087_01
Os016136_01
Os016194_01
Os016240_01
Os016301_01
Os016317_01
Os016497_01
Os016500_01
Os016575_01
Os016598_01
Os016603_01
Os016615_01
Os016642_01
Os016648_01
Os016694_01
Os016695_01
Os016713_01
Os016714_01
Os016754_01
Os016767_01
Os016789_01
Os016802_01
Os016811_01
Os016831_01
Os016845_01
Os016868_01
Os016872_01
Os016905_01
Os016917_01
Os016964_01
Os016970_01
Os016984_01
Os017002_01
Os017059_01
Os017096_01
Os017134_01
Os017149_01
Os017154_01
Os017159_01
Os017197_01
Os017233_01
Os017246_01
Os017300_01
Os017317_01
Os017335_01
Os017377_01
Os017389_01
Os017393_01
Os017401_01
Os017420_01
Os017433_01
Os017458_01
Os017514_01
Os017521_01
Os017640_01
Os017675_01
Os017747_01
Os017816_01
Os017967_01
Os018052_01
Os018069_01
Os018075_01
Os018076_01
Os018083_01
Os018108_01
Os018109_01
Os018169_01
Os018200_01
Os018203_01
Os018221_01
Os018228_01
Os018256_01
Os018287_01
Os018291_01
Os018298_01
Os018316_01
Os018329_01
Os018375_01
Os018384_01
Os018396_01
Os018397_01
Os018421_01
Os018446_01
Os018447_01
Os018452_01
Os018477_01
Os018502_01
Os018503_01
Os018540_01
Os018542_01
Os018557_01
Os018560_01
Os018580_01
Os018586_01
Os018630_01
Os018675_01
Os018679_01
Os018702_01
Os018706_01
Os018714_01
Os018788_01
Os018837_01
Os018841_01
Os018846_01
Os018849_01
Os018862_01
Os018868_01
Os018882_01
Os018887_01
Os018898_01
Os018926_01
Os018929_01
Os019071_01
Os019081_01
Os019113_01
Os019291_01
Os019330_01
Os019335_01
Os019384_01
Os019424_01
Os019426_01
Os019431_01
Os019436_01
Os019445_01
Os019469_01
Os019473_01
Os019489_01
Os019541_01
Os019580_01
Os019586_01
Os019597_01
Os019617_01
Os019619_01
Os019665_01
Os019733_01
Os019747_01
Os019765_01
Os019810_01
Os019820_01
Os019842_01
Os019898_01
Os019950_01
Os019985_01
Os020146_01
Os020227_01
Os020408_01
Os020455_01
Os020456_01
Os020472_01
Os020522_01
Os020533_01
Os020544_01
Os020576_01
Os020667_01
Os020690_01
Os020732_01
Os020733_01
Os020768_01
Os020786_01
Os020802_01
Os020816_01
Os020818_01
Os020830_01
Os020846_01
Os020868_01
Os020990_01
Os021041_01
Os021046_01
Os021048_01
Os021065_01
Os021112_01
Os021142_01
Os021176_01
Os021228_01
Os021307_01
Os021309_01
Os021315_01
Os021338_01
Os021411_01
Os021419_01
Os021448_01
Os021453_01
Os021461_01
Os021463_01
Os021465_01
Os021495_01
Os021534_01
Os021538_01
Os021588_01
Os021633_01
Os021668_01
Os021685_01
Os021784_01
Os021805_01
Os021861_01
Os021876_01
Os021888_01
Os021890_01
Os021900_01
Os021916_01
Os021927_01
Os021994_01
Os021995_01
Os021997_01
Os022015_01
Os022019_01
Os022033_01
Os022042_01
Os022046_01
Os022058_01
Os022068_01
Os022074_01
Os022084_01
Os022154_01
Os022162_01
Os022194_01
Os022234_01
Os022278_01
Os022287_01
Os022400_01
Os022460_01
Os022502_01
Os022549_01
Os022632_01
Os022657_01
Os022690_01
Os022753_01
Os022768_01
Os022863_01
Os022874_01
Os022899_01
Os022938_01
Os022940_01
Os023096_01
Os023157_01
Os023210_01
Os023212_01
Os023230_01
Os023246_01
Os023269_01
Os023323_01
Os023764_01
Os023885_01
Os023966_01
Os024025_01
Os024100_01
Os024135_01
Os024247_01
Os024310_01
Os024346_01
Os024428_01
Os024591_01
Os024620_01
Os024622_01
Os024653_01
Os024660_01
Os024678_01
Os024726_01
Os024739_01
Os024745_01
Os024774_01
Os024784_01
Os024787_01
Os024795_01
Os024799_01
Os024866_01
Os024931_01
Os024975_01
Os024992_01
Os025006_01
Os025031_01
Os025081_01
Os025084_01
Os025109_01
Os025115_01
Os025123_01
Os025138_01
Os025208_01
Os025210_01
Os025228_01
Os025297_01
Os025522_01
Os025681_01
Os025711_01
Os025793_01
Os025846_01
Os025876_01
Os025882_01
Os025904_01
Os025927_01
Os025931_01
Os026166_01
Os026209_01
Os026272_01
Os026365_01
Os026371_01
Os026405_01
Os026440_01
Os026457_01
Os026559_01
Os027173_01
Os027364_01
Os027379_02
Os027501_01
Os027504_01
Os027599_01
Os027607_01
Os027615_01
Os027647_01
Os027710_01
Os027781_01
Os027835_01
Os027843_01
Os027849_01
Os027871_01
Os027875_01
Os027924_01
Os028021_01
Os028107_01
Os028138_01
Os028158_01
Os028169_01
Os028324_01
Os028359_01
Os028374_01
Os028377_01
Os028440_01
Os028478_01
Os028483_01
Os028573_01
Os028593_01
Os028616_01
Os028620_01
Os028742_01
Os028763_01
Os028772_01
Os028778_01
Os028787_01
Os028815_01
Os028886_01
Os028931_01
Os028971_01
Os028975_01
Os028993_01
Os029012_01
Os029112_01
Os029463_01
Os029577_01
Os030020_01
Os030179_01
Os030190_01
Os030245_01
Os030427_01
Os030428_01
Os030763_01
Os030776_01
Os030880_01
Os030957_01
Os031095_01
Os031408_01
Os031418_01
Os031617_01
Os031751_01
Os031851_01
Os032172_01
Os032296_01
Os032313_01
Os032356_01
Os032360_01
Os032577_01
Os032707_01
Os032750_01
Os032882_01
Os032958_01
Os033329_01
Os033387_01
Os033533_01
Os033534_01
Os033604_01
Os033855_01
Os033897_01
Os034112_01
Os034465_01
Os034492_01
Os034520_01
Os034591_01
Os034644_01
Os034674_01
Os034737_01
Os034904_01
Os034905_01
Os034944_01
Os035267_01
Os035411_01
Os035485_01
Os035517_01
Os035689_01
Os035712_01
Os035930_01
Os035978_01
Os036058_01
Os036121_01
Os036266_01
Os036311_01
Os036343_01
Os036358_01
Os036410_01
Os036428_01
Os036453_01
Os036501_01
Os036560_01
Os036576_01
Os036618_01
Os036729_01
Os036804_01
Os036833_01
Os036979_01
Os037155_01
Os037261_01
Os037351_01
Os037447_01
Os037525_01
Os037801_01
Os037805_01
Os037878_01
Os037883_01
Os037896_01
Os037934_01
Os037973_01
Os037986_01
Os038003_01
Os038040_01
Os038086_01
Os038185_01
Os038273_01
Os038306_01
Os038403_01
Os038470_01
Os038478_01
Os038486_01
Os038640_01
Os038717_01
Os038781_01
Os038959_01
Os039006_01
Os039304_01
Os039387_01
Os039419_01
Os039506_01
Os039707_01
Os039715_01
Os039731_01
Os039774_01
Os039778_01
Os039873_01
Os039914_01
Os039921_01
Os039988_01
Os040087_01
Os040099_01
Os040185_01
Os040248_01
Os040260_01
Os040340_01
Os040465_01
Os040523_01
Os040532_01
Os040652_01
Os040660_01
Os040663_01
Os040718_01
Os040817_01
Os040846_01
Os040899_01
Os040998_01
Os041128_01
Os041196_01
Os041245_01
Os041261_01
Os041291_01
Os041369_01
Os041416_01
Os041439_01
Os041601_01
Os041744_01
Os041780_01
Os041956_01
Os042038_01
Os042266_01
Os042302_01
Os042412_01
Os042434_01
Os042508_01
Os042560_01
Os042794_01
Os042885_01
Os042940_01
Os042952_01
Os043040_01
Os043104_01
Os043416_01
Os043477_01
Os043501_01
Os043619_01
Os043894_01
Os043930_01
Os043949_01
Os043985_01
Os044043_01
Os044237_01
Os044261_01
Os044269_01
Os044274_01
Os044285_01
Os044301_01
Os044381_01
Os044439_01
Os044457_01
Os044481_01
Os044501_01
Os044526_01
Os044546_01
Os044574_01
Os044589_01
Os044797_01
Os044855_01
Os044856_01
Os044915_01
Os044959_01
Os045086_01
Os045241_01
Os045275_01
Os045285_01
Os045461_01
Os045512_01
Os045677_01
Os045694_01
Os045757_01
Os045778_01
Os045785_01
Os045912_01
Os045924_01
Os046039_01
Os046108_01
Os046187_01
Os046337_01
Os046541_01
Os046564_01
Os046568_01
Os046641_01
Os046697_01
Os046780_01
Os046974_01
Os047011_01
Os047014_01
Os047237_01
Os047350_01
Os047353_01
Os047362_01
Os047402_01
Os047560_01
Os047563_01
Os047568_01
Os047681_01
Os047711_01
Os047713_01
Os047785_01
Os047974_01
Os048003_01
Os048165_01
Os048671_01
Os048676_01
Os048711_01
Os048934_01
Os049037_01
Os049227_01
Os049228_01
Os049587_01
Os049761_01
Os050062_01
Os050157_01
Os050288_01
Os050478_01
Os050700_01
Os050864_01
Os050874_01
Os051035_01
Os051051_01
Os051156_01
Os051388_01
Os051547_01
Os051638_01
Os051701_01
Os051842_01
Os051948_01
Os051951_01
Os051956_01
Os051973_01
Os051991_01
Os051998_01
Os052018_01
Os052023_01
Os052028_01
Os052052_01
Os052085_01
Os052088_01
Os052118_01
Os052123_01
Os052145_01
Os052151_01
Os052207_01
Os052213_01
Os052217_01
Os052323_01
Os052344_01
Os052397_01
Os052431_01
Os052471_01
Os052516_01
Os052560_01
Os052565_01
Os052591_01
Os052619_01
Os052626_01
Os052659_01
Os052667_01
Os052670_01
Os052700_01
Os052749_01
Os052762_01
Os052788_01
Os052839_01
Os052880_01
Os052893_01
Os052936_01
Os052982_01
Os053032_01
Os053072_01
Os053088_01
Os053093_01
Os053135_01
Os053140_01
Os053152_01
Os053166_01
Os053194_01
Os053204_01
Os053215_01
Os053274_01
Os053321_01
Os053353_01
Os053506_01
Os053523_01
Os053534_01
Os053631_01
Os053706_01
Os053799_01
Os053866_01
Os053882_01
Os053884_01
Os053940_01
Os053941_01
Os053963_01
Os053976_01
Os053987_01
Os053989_01
Os054039_01
Os054057_01
Os054079_01
Os054086_02
Os054089_01
Os054090_01
Os054160_01
Os054358_01
Os054366_01
Os054368_01
Os054375_01
Os054412_01
Os054419_01
Os054432_01
Os054447_01
Os054452_01
Os054456_01
Os054459_01
Os054468_01
Os054492_01
Os054509_01
Os054533_01
Os054539_01
Os054561_01
Os054612_01
Os054627_01
Os054736_01
Os054741_01
Os054752_01
Os054806_01
Os054902_01
Os054911_01
Os054961_01
Os054964_01
Os054984_01
Os055041_01
Os055053_01
Os055085_01
Os055096_01
Os055145_01
Os055154_01
Os055191_01
Os055198_01
Os055199_01
Os055200_01
Os055222_01
Os055245_01
Os055247_01
Os055250_01
Os055260_01
Os055303_01
Os055323_01
Os055342_01
Os055344_01
Os055351_01
Os055372_01
Os055384_01
Os055399_01
Os055447_01
Os055529_01
Os055569_01
Os055603_01
Os055623_01
Os055681_01
Os055683_01
Os055722_01
Os055757_01
Os055758_01
Os055761_01
Os055780_01
Os055911_01
Os055978_01
Os056047_01
Os056074_02
Os056092_01
Os056132_01
Os056170_01
Os056218_02
Os056254_01
Os056269_01
Os056274_01
Os056301_01
Os056342_01
Os056344_01
Os056367_01
Os056368_01
Os056387_01
Os056391_01
Os056412_01
Os056446_01
Os056506_01
Os056518_01
Os056527_01
Os056569_01
Os056589_01
Os056602_01
Os056608_01
Os056619_01
Os056622_02
Os056624_01
Os056660_01
Os056663_01
Os056674_01
Os056695_01
Os056700_01
Os056714_01
Os056732_01
Os056739_01
Os056753_01
Os056755_01
Os056763_01
Os056781_01
Os056824_01
Os056851_01
Os056869_01
Os056897_01
Os056903_02
Os056910_02
Os056919_02
Os056934_02
Os056936_01
Os056974_02
Os056989_02
Os057015_02
Os057094_01
Os057095_02
Os057122_02
Os057161_02
Os057165_02
Os057226_02
Os057238_02
Os057254_02
Os057257_02
Os057340_02
Os057361_02
Os057451_02
Os057502_02
Os057570_02
Os057633_02

SUPPLEMENTAL TABLE 9
Oligo ID
Os000030_01
Os000043_01
Os000169_01
Os000174_01
Os000241_01
Os000251_01
Os000312_01
Os000314_01
Os000340_01
Os000381_01
Os000389_01
Os000393_01
Os000431_01
Os000508_01
Os000532_01
Os000545_01
Os000590_01
Os000597_01
Os000607_01
Os000664_01
Os000669_01
Os000757_01
Os000836_01
Os001035_01
Os001087_01
Os001238_01
Os001272_01
Os001277_01
Os001296_01
Os001300_02
Os001377_01
Os001529_01
Os001537_01
Os001555_01
Os001579_01
Os001620_01
Os001621_01
Os001649_01
Os001778_01
Os001806_01
Os001830_01
Os001842_01
Os001884_01
Os001930_01
Os001941_01
Os002028_01
Os002152_01
Os002160_01
Os002358_01
Os002390_01
Os002458_01
Os002550_01
Os002652_01
Os002696_01
Os003025_01
Os003026_01
Os003060_01
Os003284_01
Os003443_01
Os003485_01
Os003719_01
Os003794_01
Os004668_01
Os005159_01
Os005810_01
Os006070_01
Os006546_02
Os007068_01
Os007216_01
Os008023_01
Os008400_01
Os008475_01
Os008495_01
Os008548_01
Os008555_01
Os008566_01
Os008629_01
Os008639_01
Os008648_01
Os008690_01
Os008728_01
Os008761_01
Os008777_01
Os008795_01
Os008842_01
Os008956_01
Os009011_01
Os009016_01
Os009047_01
Os009117_01
Os009173_01
Os009192_01
Os009222_01
Os009231_01
Os009246_01
Os009247_01
Os009303_01
Os009338_01
Os009365_01
Os009556_01
Os009629_01
Os009637_01
Os009661_01
Os009679_01
Os009705_01
Os009744_01
Os009764_01
Os009821_01
Os009822_01
Os009858_01
Os009927_01
Os009938_01
Os009961_01
Os009984_01
Os009986_01
Os009991_01
Os009997_01
Os010011_01
Os010064_01
Os010145_01
Os010171_01
Os010206_01
Os010219_01
Os010242_01
Os010272_01
Os010278_01
Os010290_01
Os010372_01
Os010486_01
Os010510_01
Os010599_01
Os010610_01
Os010621_01
Os010685_01
Os010789_01
Os010791_01
Os010809_01
Os010826_01
Os010835_01
Os010861_01
Os010863_01
Os010864_01
Os010880_01
Os010885_01
Os010912_01
Os010924_01
Os010927_01
Os010941_01
Os010953_01
Os010959_01
Os011001_01
Os011045_01
Os011080_01
Os011103_01
Os011125_01
Os011134_01
Os011195_01
Os011231_01
Os011247_01
Os011249_01
Os011252_01
Os011290_01
Os011311_01
Os011350_01
Os011359_01
Os011414_01
Os011435_01
Os011563_01
Os011610_01
Os011785_01
Os011789_01
Os011812_01
Os011838_01
Os011851_01
Os011870_01
Os011871_01
Os011930_01
Os011972_01
Os011982_01
Os012035_01
Os012153_01
Os012157_01
Os012160_01
Os012175_01
Os012193_01
Os012245_01
Os012444_01
Os012472_01
Os012479_01
Os012497_01
Os012505_01
Os012533_01
Os012619_01
Os012623_01
Os012632_01
Os012645_01
Os012731_01
Os012795_01
Os012900_01
Os012925_01
Os012927_01
Os012955_01
Os013006_01
Os013023_01
Os013024_01
Os013030_01
Os013036_01
Os013103_01
Os013113_01
Os013114_01
Os013161_01
Os013186_01
Os013192_01
Os013210_01
Os013346_01
Os013432_01
Os013447_01
Os013473_01
Os013504_01
Os013518_01
Os013591_01
Os013659_01
Os013676_01
Os013749_01
Os013812_01
Os013838_01
Os013841_01
Os013849_01
Os013867_01
Os013874_01
Os013986_01
Os014026_01
Os014063_01
Os014108_01
Os014141_01
Os014154_01
Os014157_01
Os014197_01
Os014265_01
Os014317_01
Os014327_01
Os014360_01
Os014441_01
Os014442_01
Os014467_01
Os014505_01
Os014604_01
Os014634_01
Os014704_01
Os014717_01
Os014766_01
Os014773_01
Os014803_01
Os014836_01
Os014842_01
Os014927_01
Os014944_01
Os014962_01
Os014967_01
Os015085_01
Os015152_01
Os015245_01
Os015317_01
Os015465_01
Os015581_01
Os015589_01
Os015599_01
Os015601_01
Os015629_01
Os015638_01
Os015661_01
Os015684_01
Os015702_01
Os015790_01
Os015843_01
Os015860_01
Os015895_01
Os015910_01
Os015916_01
Os015934_01
Os015961_01
Os015995_01
Os015998_01
Os016064_01
Os016133_01
Os016141_01
Os016225_01
Os016235_01
Os016243_01
Os016305_01
Os016453_01
Os016461_01
Os016479_01
Os016486_01
Os016505_01
Os016536_01
Os016593_01
Os016599_01
Os016638_01
Os016652_01
Os016683_01
Os016716_01
Os016744_01
Os016757_01
Os016780_01
Os016793_01
Os016826_01
Os016882_01
Os016888_01
Os016901_01
Os016919_01
Os016940_01
Os017003_01
Os017037_01
Os017053_01
Os017054_01
Os017056_01
Os017108_01
Os017116_01
Os017163_01
Os017225_01
Os017243_01
Os017245_01
Os017289_01
Os017290_01
Os017334_01
Os017376_01
Os017440_01
Os017461_01
Os017489_01
Os017526_01
Os017527_01
Os017624_01
Os017628_01
Os017629_01
Os017653_01
Os017710_01
Os017726_01
Os017764_01
Os017777_01
Os017813_01
Os017890_01
Os017893_01
Os017943_01
Os017954_01
Os018042_01
Os018078_01
Os018098_01
Os018144_01
Os018147_01
Os018201_01
Os018211_01
Os018311_01
Os018314_01
Os018334_01
Os018341_01
Os018362_01
Os018381_01
Os018417_01
Os018470_01
Os018689_01
Os018734_01
Os018739_01
Os018757_01
Os018762_01
Os018786_01
Os018863_01
Os018909_01
Os018916_01
Os019010_01
Os019022_01
Os019145_01
Os019159_01
Os019194_01
Os019296_01
Os019313_01
Os019350_01
Os019364_01
Os019439_01
Os019516_01
Os019536_01
Os019540_01
Os019547_01
Os019580_01
Os019600_01
Os019627_01
Os019792_01
Os019825_01
Os019855_01
Os019891_01
Os019901_01
Os020117_01
Os020130_01
Os020216_01
Os020217_01
Os020261_01
Os020316_01
Os020340_01
Os020360_01
Os020397_01
Os020412_01
Os020424_01
Os020481_01
Os020529_01
Os020633_01
Os020653_01
Os020694_01
Os020769_01
Os020782_01
Os020807_01
Os020903_01
Os020923_01
Os020934_01
Os020962_01
Os020973_01
Os020989_01
Os021097_01
Os021156_01
Os021165_01
Os021198_01
Os021231_01
Os021293_01
Os021372_01
Os021383_01
Os021455_01
Os021490_01
Os021509_01
Os021514_01
Os021547_01
Os021555_01
Os021580_01
Os021597_01
Os021604_01
Os021615_01
Os021632_01
Os021642_01
Os021643_01
Os021705_01
Os021726_01
Os021735_01
Os021826_01
Os021843_01
Os021889_01
Os021942_01
Os021983_01
Os021996_01
Os022063_01
Os022075_01
Os022116_01
Os022133_01
Os022142_01
Os022161_01
Os022167_01
Os022209_01
Os022235_01
Os022239_01
Os022272_01
Os022279_01
Os022314_01
Os022404_01
Os022421_01
Os022431_01
Os022433_01
Os022746_01
Os022756_01
Os022790_01
Os022845_01
Os022866_01
Os022876_01
Os022919_01
Os022927_01
Os022932_01
Os022955_01
Os022976_01
Os022981_01
Os023018_01
Os023044_01
Os023056_01
Os023060_01
Os023116_01
Os023136_01
Os023350_01
Os023419_01
Os023435_01
Os023460_01
Os023465_01
Os023471_01
Os023508_01
Os023546_01
Os023582_01
Os023695_01
Os023696_01
Os023706_01
Os023709_01
Os023755_01
Os023774_01
Os023781_01
Os023810_01
Os023828_01
Os023833_01
Os023851_01
Os023944_01
Os023958_01
Os024014_01
Os024047_01
Os024115_01
Os024220_01
Os024232_01
Os024249_01
Os024254_01
Os024260_01
Os024289_01
Os024292_01
Os024307_01
Os024309_01
Os024378_01
Os024415_01
Os024474_01
Os024541_01
Os024549_01
Os024559_01
Os024563_01
Os024577_01
Os024580_01
Os024836_01
Os024867_01
Os024899_01
Os024934_01
Os025011_01
Os025013_01
Os025173_01
Os025184_01
Os025626_01
Os025638_01
Os025683_01
Os025844_01
Os025848_01
Os025984_01
Os025997_01
Os026012_01
Os026060_01
Os026142_01
Os026302_01
Os026333_01
Os026341_01
Os026346_01
Os026376_01
Os026390_01
Os026674_01
Os026758_01
Os026794_01
Os026917_01
Os027198_02
Os027591_01
Os027602_01
Os027606_01
Os027668_01
Os027869_01
Os027989_01
Os028061_01
Os028229_01
Os028491_01
Os028577_01
Os029023_01
Os029226_01
Os029244_01
Os029518_01
Os029620_01
Os029636_01
Os029736_01
Os029762_01
Os029768_01
Os030104_01
Os030254_01
Os030255_01
Os030274_01
Os030331_01
Os030343_01
Os030366_01
Os030368_01
Os030404_01
Os030719_01
Os030724_01
Os030861_01
Os030891_01
Os031053_01
Os031326_01
Os031379_01
Os032086_01
Os032266_01
Os032429_01
Os032515_01
Os032584_01
Os032875_01
Os033293_01
Os033403_01
Os033492_01
Os033716_01
Os033717_01
Os033901_01
Os033930_01
Os034076_01
Os034173_01
Os034381_01
Os034787_01
Os034833_01
Os034995_01
Os035044_01
Os035258_01
Os035594_01
Os035629_01
Os035695_01
Os035949_01
Os036145_01
Os036190_01
Os036286_01
Os036331_01
Os036573_01
Os036725_01
Os036839_01
Os036886_01
Os037056_01
Os037113_01
Os037163_01
Os037250_01
Os037577_01
Os037619_01
Os037825_01
Os038174_01
Os038407_01
Os038430_01
Os038466_01
Os038598_01
Os038926_01
Os039430_01
Os039501_01
Os039505_01
Os039696_01
Os039814_01
Os040123_01
Os040317_01
Os040345_01
Os040349_01
Os040920_01
Os041003_01
Os041201_01
Os041519_01
Os041643_01
Os041755_01
Os041962_01
Os042179_01
Os042182_01
Os042285_01
Os042707_01
Os042953_01
Os043037_01
Os043069_01
Os043315_01
Os043473_01
Os043506_01
Os043939_01
Os044090_01
Os044270_01
Os044364_01
Os044369_01
Os044667_01
Os045331_01
Os045654_01
Os045704_01
Os045929_01
Os045964_01
Os046047_01
Os046223_01
Os046257_01
Os046324_01
Os046599_01
Os046646_01
Os046795_01
Os046811_01
Os047164_01
Os047397_01
Os047919_01
Os048156_01
Os048275_01
Os049012_01
Os049100_01
Os049297_01
Os049899_01
Os049956_01
Os050241_01
Os050627_01
Os050770_01
Os051755_01
Os051804_01
Os051856_01
Os051905_01
Os051935_01
Os051974_01
Os051981_01
Os052000_01
Os052139_01
Os052148_01
Os052160_01
Os052175_01
Os052195_01
Os052198_01
Os052225_01
Os052240_01
Os052361_01
Os052387_01
Os052419_01
Os052483_01
Os052593_01
Os052594_01
Os052669_01
Os052675_01
Os052686_01
Os052782_02
Os052816_01
Os052844_01
Os052911_01
Os053018_01
Os053034_01
Os053038_01
Os053043_01
Os053057_01
Os053136_01
Os053206_01
Os053249_01
Os053316_01
Os053503_01
Os053510_01
Os053731_02
Os053746_01
Os053954_01
Os054013_01
Os054035_01
Os054037_01
Os054086_02
Os054377_01
Os054428_01
Os054430_01
Os054431_01
Os054469_01
Os054556_01
Os054562_01
Os054564_01
Os054570_01
Os054586_01
Os054590_01
Os054602_01
Os054621_01
Os054634_01
Os054637_01
Os054639_01
Os054680_01
Os054684_01
Os054694_01
Os054699_01
Os054700_01
Os054727_01
Os054731_01
Os054741_01
Os054743_01
Os054809_01
Os054817_01
Os054824_01
Os054830_01
Os054867_01
Os054889_01
Os054898_01
Os054922_01
Os054951_01
Os054974_01
Os054996_01
Os055029_01
Os055094_01
Os055103_01
Os055156_01
Os055171_01
Os055190_01
Os055242_01
Os055251_01
Os055359_01
Os055421_01
Os055470_01
Os055504_01
Os055624_01
Os055638_01
Os055708_01
Os055713_01
Os055763_01
Os055962_01
Os056035_01
Os056091_01
Os056123_01
Os056145_01
Os056162_01
Os056250_01
Os056260_01
Os056390_01
Os056424_01
Os056527_01
Os056928_02
Os056974_02
Os056998_02
Os057045_02
Os057402_02
Os057433_02
Os057436_02
Os057467_02
Os057481_02
Os057491_02
Os057552_02
Os057570_02
Os057619_02
Os057659_02

SUPPLEMENTAL TABLE 10
Oligo ID
Os000005_01
Os000007_01
Os000011_01
Os000013_01
Os000040_01
Os000046_01
Os000062_01
Os000065_01
Os000076_01
Os000079_01
Os000102_01
Os000106_01
Os000116_01
Os000129_01
Os000133_01
Os000139_01
Os000143_01
Os000163_01
Os000182_01
Os000199_01
Os000205_01
Os000208_01
Os000210_01
Os000231_01
Os000238_01
Os000268_01
Os000283_01
Os000300_01
Os000325_01
Os000333_01
Os000336_01
Os000357_01
Os000379_01
Os000380_01
Os000382_01
Os000385_01
Os000403_01
Os000411_01
Os000416_01
Os000424_01
Os000427_01
Os000435_01
Os000437_01
Os000439_01
Os000443_01
Os000446_01
Os000463_01
Os000489_01
Os000576_01
Os000584_01
Os000604_01
Os000635_01
Os000654_01
Os000698_01
Os000716_01
Os000718_01
Os000719_01
Os000755_01
Os000761_01
Os000842_01
Os000874_01
Os000931_01
Os000957_01
Os000979_01
Os000980_01
Os000994_01
Os000995_01
Os001041_01
Os001054_01
Os001081_01
Os001141_01
Os001164_01
Os001234_01
Os001284_01
Os001387_01
Os001443_01
Os001544_01
Os001573_01
Os001701_01
Os001710_01
Os001713_01
Os001752_01
Os001877_01
Os001940_01
Os002170_01
Os002528_01
Os002827_01
Os002918_01
Os002973_01
Os003180_01
Os003565_01
Os003577_01
Os003706_01
Os004074_01
Os004097_01
Os004155_01
Os004255_01
Os004292_01
Os004293_01
Os004469_01
Os004485_01
Os004540_01
Os004630_01
Os004664_01
Os004674_01
Os004678_01
Os004718_01
Os004962_01
Os005019_01
Os005027_01
Os005140_01
Os005179_01
Os005360_02
Os005402_01
Os005809_01
Os005882_01
Os005904_01
Os005906_01
Os005908_01
Os005956_01
Os006041_01
Os006132_01
Os006143_01
Os006209_01
Os006367_01
Os006382_01
Os006479_01
Os006485_01
Os006653_01
Os006670_01
Os006733_01
Os006743_01
Os006778_01
Os007107_01
Os007130_01
Os007265_01
Os007271_01
Os007284_01
Os007392_01
Os007610_02
Os007873_01
Os008046_01
Os008159_01
Os008333_01
Os008355_02
Os008364_01
Os008424_01
Os008425_01
Os008438_01
Os008463_01
Os008508_01
Os008551_01
Os008564_01
Os008572_01
Os008584_01
Os008596_01
Os008607_01
Os008647_01
Os008657_01
Os008668_01
Os008681_01
Os008685_01
Os008781_01
Os008790_01
Os008806_01
Os008824_01
Os008841_01
Os008844_01
Os008878_01
Os008906_01
Os008908_01
Os008911_01
Os008912_01
Os008930_01
Os008937_01
Os008985_01
Os008998_01
Os009005_01
Os009007_01
Os009039_01
Os009040_01
Os009058_01
Os009072_01
Os009088_01
Os009096_01
Os009097_01
Os009102_01
Os009108_01
Os009126_01
Os009138_01
Os009146_01
Os009157_01
Os009163_01
Os009177_01
Os009187_01
Os009256_01
Os009262_01
Os009280_01
Os009282_01
Os009284_01
Os009286_01
Os009291_01
Os009293_01
Os009300_01
Os009342_01
Os009348_01
Os009354_01
Os009359_01
Os009361_01
Os009461_01
Os009470_01
Os009507_01
Os009523_01
Os009535_01
Os009586_01
Os009626_01
Os009632_01
Os009643_01
Os009645_01
Os009658_01
Os009721_01
Os009723_01
Os009726_01
Os009755_01
Os009792_01
Os009793_01
Os009807_01
Os009822_01
Os009837_01
Os009854_01
Os009885_01
Os009899_01
Os009909_01
Os009920_01
Os009922_01
Os009949_01
Os009980_01
Os009989_01
Os009993_01
Os010018_01
Os010077_01
Os010114_01
Os010140_01
Os010181_01
Os010204_01
Os010214_01
Os010218_01
Os010240_01
Os010257_01
Os010339_01
Os010346_01
Os010413_01
Os010430_01
Os010444_01
Os010472_01
Os010484_01
Os010494_01
Os010513_01
Os010517_01
Os010530_01
Os010555_01
Os010600_01
Os010647_01
Os010651_01
Os010682_01
Os010683_01
Os010700_01
Os010737_01
Os010743_01
Os010745_01
Os010779_01
Os010871_01
Os010986_01
Os010993_01
Os011011_01
Os011015_01
Os011046_01
Os011064_01
Os011065_01
Os011122_01
Os011146_01
Os011183_01
Os011206_01
Os011210_01
Os011218_01
Os011273_01
Os011294_01
Os011317_01
Os011349_01
Os011543_01
Os011561_01
Os011572_01
Os011575_01
Os011616_01
Os011635_01
Os011751_01
Os011797_01
Os011805_01
Os011829_01
Os011876_01
Os011888_01
Os011892_01
Os011920_01
Os011929_01
Os011970_01
Os011974_01
Os011996_01
Os012078_01
Os012130_01
Os012140_01
Os012154_01
Os012190_01
Os012205_01
Os012220_01
Os012243_01
Os012274_01
Os012311_01
Os012320_01
Os012340_01
Os012378_01
Os012399_01
Os012409_01
Os012484_01
Os012492_01
Os012509_01
Os012544_01
Os012617_01
Os012665_01
Os012679_01
Os012691_01
Os012697_01
Os012725_01
Os012743_01
Os012748_01
Os012765_01
Os012843_01
Os012856_01
Os012864_01
Os012868_01
Os012917_01
Os012923_01
Os012942_01
Os012955_01
Os012964_01
Os012969_01
Os012974_01
Os012986_01
Os012993_01
Os013018_01
Os013022_01
Os013066_01
Os013095_01
Os013123_01
Os013152_01
Os013161_01
Os013166_01
Os013184_01
Os013201_01
Os013203_01
Os013209_01
Os013231_01
Os013279_01
Os013285_01
Os013290_01
Os013311_01
Os013341_01
Os013345_01
Os013354_01
Os013358_01
Os013382_01
Os013391_01
Os013416_01
Os013438_01
Os013443_01
Os013469_01
Os013478_01
Os013525_01
Os013548_01
Os013571_01
Os013595_01
Os013609_01
Os013618_01
Os013645_01
Os013717_01
Os013719_01
Os013725_01
Os013739_01
Os013745_01
Os013802_01
Os013845_01
Os013855_01
Os013908_01
Os013995_01
Os014028_01
Os014059_01
Os014064_01
Os014075_01
Os014123_01
Os014177_01
Os014198_01
Os014233_01
Os014270_01
Os014273_01
Os014281_01
Os014333_01
Os014375_01
Os014436_01
Os014488_01
Os014530_01
Os014572_01
Os014573_01
Os014595_01
Os014629_01
Os014652_01
Os014673_01
Os014679_01
Os014690_01
Os014779_01
Os014781_01
Os014800_01
Os014859_01
Os014929_01
Os014955_01
Os014973_01
Os015035_01
Os015056_01
Os015087_01
Os015088_01
Os015096_01
Os015117_01
Os015125_01
Os015434_01
Os015540_01
Os015558_01
Os015565_01
Os015604_01
Os015630_01
Os015658_01
Os015664_01
Os015681_01
Os015737_01
Os015757_01
Os015768_01
Os015785_01
Os015822_01
Os015888_01
Os015903_01
Os015907_01
Os015914_01
Os015922_01
Os015959_01
Os016006_01
Os016024_01
Os016051_01
Os016053_01
Os016074_01
Os016091_01
Os016094_01
Os016158_01
Os016224_01
Os016324_01
Os016333_01
Os016337_01
Os016358_01
Os016411_01
Os016419_01
Os016480_01
Os016493_01
Os016508_01
Os016598_01
Os016601_01
Os016610_01
Os016615_01
Os016636_01
Os016637_01
Os016661_01
Os016663_01
Os016666_01
Os016667_01
Os016699_01
Os016703_01
Os016767_01
Os016831_01
Os016854_01
Os016874_01
Os016876_01
Os016900_01
Os016964_01
Os016982_01
Os017033_01
Os017044_01
Os017090_01
Os017093_01
Os017111_01
Os017160_01
Os017246_01
Os017248_01
Os017282_01
Os017314_01
Os017320_01
Os017324_01
Os017335_01
Os017379_01
Os017399_01
Os017416_01
Os017448_01
Os017488_01
Os017530_01
Os017555_01
Os017636_01
Os017697_01
Os017698_01
Os017722_01
Os017741_01
Os017744_01
Os017746_01
Os017761_01
Os017766_01
Os017805_01
Os017825_01
Os017848_01
Os017857_01
Os017858_01
Os017874_01
Os017875_01
Os017944_01
Os017956_01
Os017960_01
Os017990_01
Os018009_01
Os018028_01
Os018057_01
Os018095_01
Os018099_01
Os018102_01
Os018103_01
Os018124_01
Os018140_01
Os018158_01
Os018166_01
Os018176_01
Os018181_01
Os018196_01
Os018232_01
Os018261_01
Os018284_01
Os018325_01
Os018344_01
Os018365_01
Os018422_01
Os018469_01
Os018473_01
Os018519_01
Os018535_01
Os018550_01
Os018557_01
Os018574_01
Os018631_01
Os018638_01
Os018647_01
Os018675_01
Os018708_01
Os018724_01
Os018736_01
Os018750_01
Os018783_01
Os018787_01
Os018798_01
Os018810_01
Os018821_01
Os018915_01
Os018919_01
Os018928_01
Os018992_01
Os018994_01
Os019013_01
Os019014_01
Os019018_01
Os019054_01
Os019058_01
Os019070_01
Os019112_01
Os019137_01
Os019140_01
Os019141_01
Os019144_01
Os019182_01
Os019183_01
Os019188_01
Os019193_01
Os019268_01
Os019300_01
Os019311_01
Os019312_01
Os019320_01
Os019334_01
Os019371_01
Os019381_01
Os019387_01
Os019413_01
Os019423_01
Os019426_01
Os019445_01
Os019534_01
Os019568_01
Os019571_01
Os019575_01
Os019589_01
Os019655_01
Os019668_01
Os019703_01
Os019704_01
Os019705_01
Os019724_01
Os019747_01
Os019776_01
Os019781_01
Os019790_01
Os019828_01
Os019888_01
Os019910_01
Os019916_01
Os019952_01
Os019953_01
Os019963_01
Os019969_01
Os019983_01
Os019993_01
Os020020_01
Os020027_01
Os020072_01
Os020111_01
Os020144_01
Os020152_01
Os020170_01
Os020190_01
Os020195_01
Os020197_01
Os020208_01
Os020214_01
Os020225_01
Os020235_01
Os020243_01
Os020253_01
Os020313_01
Os020322_01
Os020377_01
Os020383_01
Os020394_01
Os020429_01
Os020448_01
Os020464_01
Os020480_01
Os020493_01
Os020499_01
Os020500_01
Os020556_01
Os020560_01
Os020572_01
Os020576_01
Os020588_01
Os020603_01
Os020608_01
Os020627_01
Os020637_01
Os020666_01
Os020712_01
Os020730_01
Os020740_01
Os020743_01
Os020763_01
Os020790_01
Os020817_01
Os020818_01
Os020842_01
Os020843_01
Os020846_01
Os020865_01
Os020905_01
Os020929_01
Os020937_01
Os020943_01
Os020970_01
Os020978_01
Os021017_01
Os021044_01
Os021065_01
Os021074_01
Os021101_01
Os021151_01
Os021183_01
Os021185_01
Os021189_01
Os021205_01
Os021211_01
Os021213_01
Os021220_01
Os021223_01
Os021305_01
Os021338_01
Os021348_01
Os021410_01
Os021414_01
Os021438_01
Os021457_01
Os021463_01
Os021467_01
Os021473_01
Os021494_01
Os021510_01
Os021521_01
Os021550_01
Os021609_01
Os021616_01
Os021649_01
Os021670_01
Os021674_01
Os021685_01
Os021697_01
Os021747_01
Os021754_01
Os021783_01
Os021844_01
Os021896_01
Os021919_01
Os021934_01
Os021939_01
Os021949_01
Os021973_01
Os021974_01
Os022005_01
Os022020_01
Os022047_01
Os022084_01
Os022128_01
Os022134_01
Os022135_01
Os022156_01
Os022228_01
Os022263_01
Os022368_01
Os022389_01
Os022399_01
Os022445_01
Os022447_01
Os022456_01
Os022508_01
Os022513_01
Os022564_01
Os022583_01
Os022592_01
Os022636_01
Os022675_01
Os022705_01
Os022738_01
Os022784_01
Os022920_01
Os022933_01
Os022935_01
Os022950_01
Os022956_01
Os022962_01
Os022963_01
Os023007_01
Os023011_01
Os023190_01
Os023260_01
Os023369_01
Os023426_01
Os023505_01
Os023547_01
Os023583_01
Os023835_01
Os023860_01
Os023925_01
Os023934_01
Os023966_01
Os023968_01
Os023971_01
Os024063_01
Os024102_01
Os024121_01
Os024139_01
Os024146_01
Os024172_01
Os024201_01
Os024274_01
Os024310_01
Os024428_01
Os024536_01
Os024591_01
Os024670_01
Os024733_01
Os024737_01
Os024741_01
Os024776_01
Os024783_01
Os024787_01
Os024792_01
Os024793_01
Os024826_01
Os024932_01
Os024961_01
Os024971_01
Os025106_01
Os025110_01
Os025158_01
Os025211_01
Os025217_01
Os025506_01
Os025779_01
Os025846_01
Os025876_01
Os025891_01
Os025905_01
Os025990_01
Os026064_01
Os026137_01
Os026185_01
Os026359_01
Os026407_01
Os026438_01
Os026925_01
Os027044_01
Os027312_01
Os027656_01
Os027685_01
Os027733_01
Os027801_01
Os027872_01
Os027979_01
Os028014_01
Os028041_01
Os028054_01
Os028087_01
Os028169_01
Os028400_01
Os028401_01
Os028465_01
Os028583_01
Os028597_01
Os028607_01
Os028616_01
Os028721_01
Os028744_01
Os029016_01
Os029025_01
Os029031_01
Os029049_01
Os029073_01
Os029111_01
Os029129_01
Os029141_01
Os029171_01
Os029177_01
Os029201_01
Os029208_01
Os029213_01
Os029218_01
Os029224_01
Os029227_01
Os029255_01
Os029261_01
Os029266_01
Os029272_01
Os029280_01
Os029290_01
Os029291_01
Os029296_01
Os029303_01
Os029318_01
Os029327_01
Os029333_01
Os029405_01
Os029410_01
Os029470_01
Os029486_01
Os029516_01
Os029534_01
Os029604_01
Os029605_01
Os029610_01
Os029682_01
Os029802_01
Os029814_01
Os029943_01
Os030063_01
Os030108_01
Os030125_01
Os030268_01
Os030417_01
Os030427_01
Os030489_01
Os030693_01
Os030763_01
Os030807_01
Os030881_01
Os030936_01
Os030963_01
Os030991_01
Os031053_01
Os031090_01
Os031094_01
Os031095_01
Os031141_01
Os031321_01
Os031351_01
Os031354_01
Os031359_01
Os031375_01
Os031442_01
Os031519_01
Os031528_01
Os031550_01
Os031725_01
Os031749_01
Os031750_01
Os031755_01
Os031777_01
Os031793_01
Os031851_01
Os031898_01
Os031957_01
Os031963_01
Os032041_01
Os032057_01
Os032136_01
Os032250_01
Os032304_01
Os032331_01
Os032337_01
Os032484_01
Os032519_01
Os032674_01
Os032827_01
Os032842_01
Os032882_01
Os032958_01
Os033019_01
Os033035_01
Os033246_01
Os033310_01
Os033534_01
Os033556_01
Os033576_01
Os033577_01
Os033578_01
Os033579_01
Os033582_01
Os033670_01
Os033672_01
Os033679_01
Os033700_01
Os033751_01
Os033779_01
Os033833_01
Os033860_01
Os033873_01
Os033885_01
Os033954_01
Os033962_01
Os034061_01
Os034180_01
Os034186_01
Os034193_01
Os034199_01
Os034326_01
Os034412_01
Os034417_01
Os034627_01
Os034628_01
Os034681_01
Os034852_01
Os034912_01
Os034921_01
Os035012_01
Os035048_01
Os035066_01
Os035143_01
Os035284_01
Os035299_01
Os035400_01
Os035415_01
Os035686_01
Os035714_01
Os035758_01
Os035861_01
Os035973_01
Os035999_01
Os036112_01
Os036123_01
Os036254_01
Os036561_01
Os036576_01
Os036632_01
Os036658_01
Os036751_01
Os036766_01
Os036864_01
Os037254_01
Os037398_01
Os037412_01
Os037414_01
Os037795_01
Os038137_01
Os038201_01
Os038211_01
Os038228_01
Os038332_01
Os038334_01
Os038335_01
Os038352_01
Os038480_01
Os038506_01
Os038546_01
Os038951_01
Os039062_01
Os039095_01
Os039391_01
Os039430_01
Os039476_01
Os039617_01
Os039725_01
Os039730_01
Os039737_01
Os039861_01
Os040008_01
Os040015_01
Os040016_01
Os040042_01
Os040185_01
Os040191_01
Os040248_01
Os040272_01
Os040340_01
Os040641_01
Os040696_01
Os040830_01
Os041324_01
Os041425_01
Os041472_01
Os041608_01
Os041991_01
Os041992_01
Os042012_01
Os042164_01
Os042243_01
Os042296_01
Os042441_01
Os042479_01
Os042556_01
Os042618_01
Os042694_01
Os042695_01
Os042835_01
Os042866_01
Os042952_01
Os042974_01
Os042979_01
Os042985_01
Os042998_01
Os043256_01
Os043266_01
Os043276_01
Os043354_01
Os043735_01
Os043788_01
Os043894_01
Os043897_01
Os043966_01
Os044056_01
Os044151_01
Os044155_01
Os044473_01
Os044501_01
Os044589_01
Os044640_01
Os044769_01
Os044797_01
Os044856_01
Os045027_01
Os045134_01
Os045282_01
Os045661_01
Os045747_01
Os045934_01
Os046047_01
Os046109_01
Os046556_01
Os046641_01
Os046654_01
Os046801_01
Os046825_01
Os046927_01
Os047011_01
Os047127_01
Os047453_01
Os047662_01
Os047664_01
Os047915_01
Os047974_01
Os047976_01
Os048008_01
Os048141_01
Os048165_01
Os048367_01
Os048482_01
Os049032_01
Os049037_01
Os049047_01
Os049076_01
Os049085_01
Os049303_01
Os049451_01
Os049614_01
Os049791_01
Os049881_01
Os050157_01
Os050178_01
Os050730_01
Os051240_01
Os051902_01
Os051904_01
Os051933_01
Os051936_01
Os051941_01
Os051951_01
Os051961_01
Os051962_01
Os051971_01
Os051973_01
Os051978_01
Os052003_01
Os052024_01
Os052032_02
Os052036_01
Os052052_01
Os052073_01
Os052075_01
Os052103_01
Os052104_01
Os052114_01
Os052123_01
Os052124_01
Os052146_01
Os052154_01
Os052184_01
Os052187_01
Os052241_01
Os052243_01
Os052254_01
Os052258_01
Os052262_01
Os052303_01
Os052335_01
Os052339_01
Os052344_01
Os052347_01
Os052354_01
Os052406_01
Os052431_01
Os052513_01
Os052548_02
Os052563_01
Os052591_01
Os052610_01
Os052632_01
Os052714_01
Os052715_01
Os052718_01
Os052734_01
Os052747_01
Os052776_01
Os052807_01
Os052831_01
Os052849_01
Os052858_01
Os052865_01
Os052866_01
Os052868_01
Os052896_01
Os052898_01
Os052905_01
Os052909_01
Os052911_01
Os052918_01
Os052934_01
Os052967_01
Os053072_01
Os053102_01
Os053241_01
Os053266_01
Os053368_01
Os053460_01
Os053489_01
Os053502_01
Os053523_01
Os053633_01
Os053672_01
Os053688_01
Os053705_01
Os053725_01
Os053828_01
Os053999_01
Os054060_01
Os054186_01
Os054197_01
Os054235_01
Os054237_01
Os054245_01
Os054284_01
Os054366_01
Os054377_01
Os054381_01
Os054382_01
Os054392_01
Os054442_01
Os054460_01
Os054468_01
Os054478_01
Os054489_01
Os054490_01
Os054495_01
Os054509_01
Os054518_01
Os054552_01
Os054555_01
Os054583_01
Os054588_01
Os054601_01
Os054607_01
Os054608_01
Os054610_01
Os054656_01
Os054661_02
Os054687_01
Os054726_01
Os054736_01
Os054801_01
Os054805_01
Os054820_01
Os054821_01
Os054827_01
Os054879_01
Os054883_01
Os054903_01
Os054911_01
Os054912_01
Os054924_01
Os054926_01
Os054950_01
Os054961_01
Os054975_01
Os055015_01
Os055049_01
Os055052_01
Os055098_01
Os055101_01
Os055140_01
Os055147_01
Os055155_01
Os055172_01
Os055173_01
Os055176_01
Os055198_01
Os055205_01
Os055252_01
Os055260_01
Os055290_01
Os055309_01
Os055322_01
Os055323_01
Os055324_01
Os055364_01
Os055369_01
Os055384_01
Os055403_01
Os055498_01
Os055559_01
Os055571_01
Os055577_01
Os055598_01
Os055603_01
Os055761_01
Os055860_01
Os056004_01
Os056047_01
Os056152_01
Os056170_01
Os056309_01
Os056330_01
Os056339_01
Os056341_01
Os056468_01
Os056498_01
Os056651_01
Os056686_01
Os056739_01
Os056755_01
Os056851_01
Os056897_01
Os056901_01
Os056914_02
Os056936_01
Os056954_02
Os056989_02
Os057052_02
Os057099_02
Os057157_02
Os057165_02
Os057198_02
Os057199_02
Os057204_01
Os057210_02
Os057245_02
Os057264_02
Os057291_02
Os057297_02
Os057314_02
Os057318_02
Os057451_02
Os057492_02
Os057633_02

SUPPLEMENTAL TABLE 11
Oligo ID

Os000009_01
Os000035_02
Os000074_01
Os000087_01
Os000128_01
Os000149_01
Os000164_01
Os000168_01
Os000169_01
Os000174_01
Os000180_01
Os000198_01
Os000225_01
Os000253_01
Os000273_01
Os000283_01
Os000389_01
Os000431_01
Os000479_01
Os000481_01
Os000508_01
Os000524_01
Os000532_01
Os000560_01
Os000575_01
Os000594_01
Os000597_01
Os000608_01
Os000689_01
Os000718_01
Os000719_01
Os000757_01
Os000787_01
Os000842_01
Os000867_01
Os000893_01
Os000911_01
Os000918_01
Os000942_01
Os000971_01
Os000973_01
Os000999_01
Os001004_01
Os001035_01
Os001046_01
Os001064_01
Os001072_01
Os001077_01
Os001087_01
Os001105_01
Os001127_01
Os001217_01
Os001218_01
Os001228_01
Os001300_02
Os001355_01
Os001374_01
Os001380_01
Os001421_01
Os001441_01
Os001461_01
Os001484_01
Os001522_01
Os001578_01
Os001592_02
Os001606_01
Os001620_01
Os001655_01
Os001758_01
Os001762_01
Os001786_01
Os001797_01
Os001806_01
Os001870_01
Os001896_01
Os001903_01
Os001911_01
Os001927_01
Os001934_01
Os001937_01
Os001939_01
Os001968_01
Os001979_01
Os002013_01
Os002104_02
Os002137_02
Os002152_01
Os002188_01
Os002206_01
Os002231_01
Os002300_01
Os002356_01
Os002358_01
Os002448_01
Os002481_01
Os002482_01
Os002516_02
Os002544_01
Os002550_01
Os002633_01
Os002696_01
Os002793_01
Os002903_01
Os002988_01
Os003190_01
Os003215_01
Os003257_01
Os003284_01
Os003332_01
Os003433_01
Os003512_02
Os003565_01
Os003577_01
Os003645_01
Os003828_01
Os003879_01
Os004097_01
Os004255_01
Os004298_01
Os004342_01
Os004372_01
Os004422_01
Os004463_01
Os004591_01
Os004700_01
Os004794_01
Os004857_01
Os004865_01
Os004928_01
Os005159_01
Os005351_01
Os005460_01
Os005571_01
Os005879_01
Os005941_01
Os005944_01
Os005953_01
Os005956_01
Os005967_01
Os006047_01
Os006088_01
Os006143_01
Os006333_02
Os006571_01
Os006587_01
Os006619_01
Os006698_01
Os006714_01
Os006993_01
Os007080_01
Os007216_01
Os007258_01
Os007263_01
Os007467_01
Os007500_01
Os007528_01
Os007622_01
Os007697_01
Os007729_01
Os007764_01
Os007866_01
Os007921_01
Os008081_01
Os008329_01
Os008354_01
Os008399_01
Os008400_01
Os008437_01
Os008441_01
Os008450_01
Os008460_01
Os008462_01
Os008495_01
Os008548_01
Os008555_01
Os008571_01
Os008591_01
Os008599_01
Os008604_01
Os008624_01
Os008629_01
Os008639_01
Os008642_01
Os008652_01
Os008676_01
Os008690_01
Os008691_01
Os008707_01
Os008754_01
Os008761_01
Os008773_01
Os008817_01
Os008842_01
Os008866_01
Os008895_01
Os008932_01
Os008947_01
Os008971_01
Os008973_01
Os009002_01
Os009011_01
Os009032_01
Os009044_01
Os009106_01
Os009115_01
Os009117_01
Os009131_01
Os009163_01
Os009224_01
Os009226_01
Os009268_01
Os009354_01
Os009433_01
Os009458_01
Os009468_01
Os009509_01
Os009520_01
Os009529_01
Os009550_01
Os009556_01
Os009582_01
Os009657_01
Os009661_01
Os009716_01
Os009764_01
Os009821_01
Os009858_01
Os009868_01
Os009887_01
Os009954_01
Os009984_01
Os009986_01
Os009987_01
Os010011_01
Os010042_01
Os010139_01
Os010171_01
Os010180_01
Os010191_01
Os010201_01
Os010219_01
Os010224_01
Os010246_01
Os010275_01
Os010290_01
Os010324_01
Os010339_01
Os010372_01
Os010407_01
Os010411_01
Os010442_01
Os010453_01
Os010473_01
Os010483_01
Os010486_01
Os010520_01
Os010579_01
Os010591_01
Os010594_01
Os010610_01
Os010652_01
Os010694_01
Os010728_01
Os010738_01
Os010739_01
Os010786_01
Os010789_01
Os010791_01
Os010804_01
Os010807_01
Os010814_01
Os010820_01
Os010837_01
Os010844_01
Os010858_01
Os010861_01
Os010863_01
Os010867_01
Os010878_01
Os010883_01
Os010884_01
Os010885_01
Os010889_01
Os010890_01
Os010904_01
Os010912_01
Os010924_01
Os010941_01
Os010946_01
Os010959_01
Os010968_01
Os010969_01
Os010977_01
Os011001_01
Os011003_01
Os011007_01
Os011018_01
Os011034_01
Os011035_01
Os011045_01
Os011124_01
Os011125_01
Os011129_01
Os011159_01
Os011164_01
Os011169_01
Os011176_01
Os011208_01
Os011213_01
Os011233_01
Os011249_01
Os011250_01
Os011313_01
Os011327_01
Os011410_01
Os011414_01
Os011485_01
Os011490_01
Os011521_01
Os011526_01
Os011535_01
Os011597_01
Os011607_01
Os011648_01
Os011654_01
Os011809_01
Os011810_01
Os011838_01
Os011854_01
Os011888_01
Os011926_01
Os011930_01
Os011982_01
Os011990_01
Os012035_01
Os012038_01
Os012042_01
Os012049_01
Os012088_01
Os012152_01
Os012192_01
Os012216_01
Os012245_01
Os012355_01
Os012405_01
Os012419_01
Os012497_01
Os012553_01
Os012558_01
Os012579_01
Os012619_01
Os012623_01
Os012632_01
Os012639_01
Os012723_01
Os012781_01
Os012813_01
Os012848_01
Os012865_01
Os012878_01
Os012898_01
Os012960_01
Os012991_01
Os013133_01
Os013151_01
Os013169_01
Os013186_01
Os013189_01
Os013244_01
Os013253_01
Os013284_01
Os013295_01
Os013378_01
Os013432_01
Os013462_01
Os013468_01
Os013470_01
Os013555_01
Os013562_01
Os013625_01
Os013659_01
Os013676_01
Os013723_01
Os013725_01
Os013815_01
Os013818_01
Os013876_01
Os013967_01
Os014026_01
Os014099_01
Os014119_01
Os014132_01
Os014157_01
Os014158_01
Os014183_01
Os014201_01
Os014236_01
Os014330_01
Os014392_01
Os014434_01
Os014441_01
Os014465_01
Os014505_01
Os014535_01
Os014542_01
Os014554_01
Os014555_01
Os014556_01
Os014557_01
Os014613_01
Os014634_01
Os014665_01
Os014673_01
Os014747_01
Os014771_01
Os014801_01
Os014803_01
Os014818_01
Os014874_01
Os014933_01
Os014983_01
Os015073_01
Os015074_01
Os015168_01
Os015192_01
Os015285_01
Os015308_01
Os015326_01
Os015461_01
Os015485_01
Os015500_01
Os015509_01
Os015515_01
Os015589_01
Os015599_01
Os015684_01
Os015685_01
Os015708_01
Os015717_01
Os015733_01
Os015737_01
Os015738_01
Os015790_01
Os015825_01
Os015849_01
Os015857_01
Os015886_01
Os015957_01
Os015963_01
Os015991_01
Os015996_01
Os015998_01
Os016016_01
Os016030_01
Os016038_01
Os016068_01
Os016085_01
Os016199_01
Os016212_01
Os016220_01
Os016221_01
Os016225_01
Os016235_01
Os016243_01
Os016278_01
Os016309_01
Os016313_01
Os016415_01
Os016423_01
Os016442_01
Os016476_01
Os016504_01
Os016505_01
Os016515_01
Os016516_01
Os016556_01
Os016601_01
Os016608_01
Os016638_01
Os016639_01
Os016652_01
Os016661_01
Os016672_01
Os016701_01
Os016715_01
Os016757_01
Os016769_01
Os016780_01
Os016800_01
Os016826_01
Os016853_01
Os016858_01
Os016871_01
Os016940_01
Os016946_01
Os016952_01
Os016988_01
Os017002_01
Os017034_01
Os017039_01
Os017055_01
Os017138_01
Os017163_01
Os017290_01
Os017334_01
Os017386_01
Os017440_01
Os017443_01
Os017526_01
Os017624_01
Os017629_01
Os017651_01
Os017653_01
Os017664_01
Os017669_01
Os017691_01
Os017706_01
Os017710_01
Os017730_01
Os017764_01
Os017785_01
Os017812_01
Os017865_01
Os017893_01
Os017901_01
Os017954_01
Os018004_01
Os018013_01
Os018022_01
Os018044_01
Os018049_01
Os018056_01
Os018065_01
Os018124_01
Os018173_01
Os018216_01
Os018238_01
Os018253_01
Os018266_01
Os018314_01
Os018319_01
Os018348_01
Os018376_01
Os018378_01
Os018381_01
Os018393_01
Os018427_01
Os018433_01
Os018470_01
Os018479_01
Os018538_01
Os018539_01
Os018567_01
Os018642_01
Os018707_01
Os018712_01
Os018713_01
Os018796_01
Os018886_01
Os018915_01
Os018920_01
Os018961_01
Os019077_01
Os019127_01
Os019138_01
Os019139_01
Os019292_01
Os019305_01
Os019316_01
Os019346_01
Os019389_01
Os019400_01
Os019431_01
Os019439_01
Os019492_01
Os019506_01
Os019571_01
Os019575_01
Os019652_01
Os019701_01
Os019731_01
Os019755_01
Os019825_01
Os019879_01
Os019941_01
Os019992_01
Os020008_01
Os020009_01
Os020045_01
Os020073_01
Os020090_01
Os020130_01
Os020134_01
Os020145_01
Os020216_01
Os020217_01
Os020220_01
Os020229_01
Os020249_01
Os020290_01
Os020292_01
Os020295_01
Os020360_01
Os020392_01
Os020429_01
Os020450_01
Os020523_01
Os020564_01
Os020581_01
Os020584_01
Os020627_01
Os020666_01
Os020678_01
Os020679_01
Os020687_01
Os020694_01
Os020714_01
Os020732_01
Os020769_01
Os020778_01
Os020903_01
Os020908_01
Os020931_01
Os020946_01
Os020961_01
Os021103_01
Os021108_01
Os021194_01
Os021202_01
Os021430_01
Os021445_01
Os021511_01
Os021539_01
Os021547_01
Os021552_01
Os021555_01
Os021642_01
Os021643_01
Os021661_01
Os021689_01
Os021709_01
Os021716_01
Os021723_01
Os021735_01
Os021757_01
Os021768_01
Os021776_01
Os021802_01
Os021814_01
Os021845_01
Os021850_01
Os021861_01
Os021877_01
Os021904_01
Os021912_01
Os021919_01
Os021942_01
Os022010_01
Os022057_01
Os022063_01
Os022083_01
Os022120_01
Os022141_01
Os022167_01
Os022236_01
Os022239_01
Os022248_01
Os022265_01
Os022309_01
Os022314_01
Os022318_01
Os022371_01
Os022381_01
Os022389_01
Os022404_01
Os022416_01
Os022433_01
Os022434_01
Os022492_01
Os022584_01
Os022634_01
Os022655_01
Os022669_01
Os022780_01
Os022784_01
Os022789_01
Os022806_01
Os022829_01
Os022856_01
Os022859_01
Os022866_01
Os022883_01
Os022913_01
Os022926_01
Os022927_01
Os022928_01
Os022938_01
Os022939_01
Os022943_01
Os022953_01
Os022954_01
Os022965_01
Os022978_01
Os022986_01
Os022990_01
Os022994_01
Os022995_01
Os023000_01
Os023002_01
Os023012_01
Os023013_01
Os023014_01
Os023021_01
Os023023_01
Os023024_01
Os023026_01
Os023027_01
Os023044_01
Os023048_01
Os023060_01
Os023066_01
Os023085_01
Os023098_01
Os023100_01
Os023113_01
Os023116_01
Os023118_01
Os023123_01
Os023156_01
Os023194_01
Os023197_01
Os023198_01
Os023224_01
Os023236_01
Os023250_01
Os023286_01
Os023306_01
Os023320_01
Os023337_01
Os023361_01
Os023364_01
Os023367_01
Os023378_01
Os023424_01
Os023465_01
Os023467_01
Os023486_01
Os023494_01
Os023495_01
Os023530_01
Os023575_01
Os023582_01
Os023604_01
Os023610_01
Os023619_01
Os023628_01
Os023631_01
Os023651_01
Os023652_01
Os023657_01
Os023671_01
Os023673_01
Os023677_01
Os023700_01
Os023706_01
Os023709_01
Os023756_01
Os023767_01
Os023793_01
Os023819_01
Os023820_01
Os023838_01
Os023849_01
Os023855_01
Os023875_01
Os023887_01
Os023898_01
Os023908_01
Os023933_01
Os023956_01
Os023964_01
Os024073_01
Os024109_01
Os024110_01
Os024116_01
Os024120_01
Os024139_01
Os024199_01
Os024220_01
Os024223_01
Os024239_01
Os024331_01
Os024347_01
Os024380_01
Os024403_01
Os024420_01
Os024425_01
Os024527_01
Os024549_01
Os024559_01
Os024563_01
Os024578_01
Os024580_01
Os024605_01
Os024634_01
Os024653_01
Os024674_01
Os024677_01
Os024727_01
Os024752_01
Os024803_01
Os024808_01
Os024813_01
Os024824_01
Os024827_01
Os024869_01
Os024909_01
Os024916_01
Os024925_01
Os025036_01
Os025048_01
Os025105_01
Os025108_01
Os025147_01
Os025153_01
Os025159_01
Os025178_01
Os025213_01
Os025402_01
Os025433_01
Os025629_01
Os025683_01
Os025748_01
Os025760_01
Os025795_01
Os025804_01
Os025848_01
Os025853_01
Os025872_01
Os025915_01
Os025919_01
Os025928_01
Os025992_01
Os026010_01
Os026053_01
Os026060_01
Os026088_01
Os026106_01
Os026128_01
Os026132_01
Os026166_01
Os026287_01
Os026305_01
Os026316_01
Os026390_01
Os026462_01
Os026467_01
Os026687_01
Os027096_01
Os027173_01
Os027211_01
Os027218_01
Os027495_01
Os027497_01
Os027596_01
Os027602_01
Os027641_01
Os027643_01
Os027655_01
Os027656_01
Os027666_01
Os027667_01
Os027693_01
Os027740_01
Os027783_01
Os027840_01
Os027878_01
Os027884_01
Os027914_01
Os027945_01
Os028054_01
Os028119_01
Os028124_01
Os028171_01
Os028173_01
Os028175_01
Os028183_01
Os028190_01
Os028198_01
Os028213_01
Os028220_01
Os028322_01
Os028355_01
Os028481_01
Os028550_01
Os028564_01
Os028586_01
Os028731_01
Os028862_01
Os028893_01
Os028895_01
Os028956_01
Os028957_01
Os028980_01
Os029007_01
Os029131_01
Os029141_01
Os029196_01
Os029203_01
Os029213_01
Os029273_01
Os029294_01
Os029316_01
Os029368_01
Os029379_01
Os029398_01
Os029410_01
Os029612_01
Os029664_01
Os029666_01
Os029689_01
Os029756_01
Os029812_01
Os029833_01
Os029915_01
Os029921_01
Os029927_01
Os029929_01
Os029982_01
Os029992_01
Os029996_01
Os029997_01
Os029998_01
Os030007_01
Os030022_01
Os030024_01
Os030071_01
Os030164_01
Os030173_01
Os030231_01
Os030256_01
Os030372_01
Os030757_01
Os030777_01
Os030802_01
Os030813_01
Os030831_01
Os030861_01
Os030862_01
Os030865_01
Os030956_01
Os030991_01
Os031037_01
Os031088_01
Os031091_01
Os031126_01
Os031132_01
Os031141_01
Os031218_01
Os031238_01
Os031259_01
Os031311_01
Os031320_01
Os031345_01
Os031350_01
Os031420_01
Os031429_01
Os031468_01
Os031487_01
Os031501_01
Os031525_01
Os031575_01
Os031586_01
Os031591_01
Os031593_01
Os031598_01
Os031620_01
Os031712_01
Os031927_01
Os031967_01
Os032001_01
Os032009_01
Os032197_01
Os032239_01
Os032244_01
Os032367_01
Os032387_01
Os032448_01
Os032470_01
Os032491_01
Os032530_01
Os032690_01
Os032731_01
Os032856_01
Os032999_01
Os033001_01
Os033011_01
Os033022_01
Os033181_01
Os033208_01
Os033381_01
Os033442_01
Os033590_01
Os033637_01
Os033713_01
Os033742_01
Os033752_01
Os033808_01
Os033879_01
Os033889_01
Os033917_01
Os033970_01
Os034039_01
Os034057_01
Os034173_01
Os034189_01
Os034406_01
Os034533_01
Os034542_01
Os034614_01
Os034680_01
Os034821_01
Os034833_01
Os034905_01
Os035044_01
Os035193_01
Os035240_01
Os035328_01
Os035424_01
Os035629_01
Os035657_01
Os035852_01
Os035961_01
Os036015_01
Os036030_01
Os036085_01
Os036148_01
Os036190_01
Os036348_01
Os036499_01
Os036519_01
Os036525_01
Os036670_01
Os036695_01
Os036839_01
Os036907_01
Os036926_01
Os036945_01
Os037254_01
Os037266_01
Os037267_01
Os037441_01
Os037496_01
Os037564_01
Os037719_01
Os037800_01
Os037922_01
Os037983_01
Os038089_01
Os038093_01
Os038170_01
Os038232_01
Os038428_01
Os038430_01
Os038458_01
Os038589_01
Os038616_01
Os038633_01
Os038691_01
Os038788_01
Os038841_01
Os038962_01
Os038986_01
Os039009_01
Os039064_01
Os039173_01
Os039193_01
Os039379_01
Os039406_01
Os039427_01
Os039505_01
Os039553_01
Os039556_01
Os039790_01
Os039922_01
Os040139_01
Os040381_01
Os040466_01
Os040550_01
Os040645_01
Os040887_01
Os040919_01
Os040991_01
Os041003_01
Os041007_01
Os041015_01
Os041019_01
Os041076_01
Os041166_01
Os041214_01
Os041307_01
Os041382_01
Os041515_01
Os041571_01
Os041647_01
Os041860_01
Os042131_01
Os042237_01
Os042608_01
Os042728_01
Os042873_01
Os043028_01
Os043030_01
Os043153_01
Os043220_01
Os043278_01
Os043342_01
Os043368_01
Os043569_01
Os043617_01
Os043703_01
Os043758_01
Os044300_01
Os044459_01
Os044466_01
Os044749_01
Os045167_01
Os045529_01
Os045735_01
Os045741_01
Os045744_01
Os045761_01
Os046144_01
Os046223_01
Os046257_01
Os046376_01
Os046701_01
Os047007_01
Os047276_01
Os047671_01
Os047729_01
Os047930_01
Os047942_01
Os047958_01
Os048275_01
Os048403_01
Os048508_01
Os048776_01
Os048899_01
Os048993_01
Os049116_01
Os049125_01
Os049129_01
Os049187_01
Os049228_01
Os049253_01
Os049257_01
Os049482_01
Os049551_01
Os049786_01
Os050244_01
Os050584_01
Os050663_01
Os050855_01
Os050903_01
Os051476_01
Os051515_01
Os051625_01
Os051717_01
Os051842_01
Os051887_01
Os051892_01
Os051896_01
Os051909_01
Os051912_01
Os051930_01
Os051935_01
Os051949_01
Os051974_01
Os051981_01
Os051991_01
Os051993_01
Os052000_01
Os052027_01
Os052035_01
Os052069_01
Os052100_01
Os052192_01
Os052225_01
Os052230_01
Os052239_01
Os052278_01
Os052287_01
Os052298_01
Os052307_01
Os052361_01
Os052374_02
Os052397_01
Os052483_01
Os052542_01
Os052555_01
Os052557_01
Os052669_01
Os052782_02
Os052807_01
Os052865_01
Os052872_01
Os052918_01
Os052939_01
Os053004_01
Os053139_01
Os053145_01
Os053503_01
Os053553_01
Os053731_02
Os053954_01
Os053976_01
Os054066_01
Os054316_01
Os054344_01
Os054362_01
Os054370_01
Os054371_01
Os054376_01
Os054377_01
Os054379_01
Os054380_01
Os054385_01
Os054421_01
Os054422_01
Os054428_01
Os054429_01
Os054431_01
Os054458_01
Os054477_01
Os054522_01
Os054538_01
Os054546_01
Os054557_01
Os054558_01
Os054562_01
Os054564_01
Os054571_01
Os054579_01
Os054580_01
Os054590_01
Os054609_01
Os054611_01
Os054621_01
Os054630_01
Os054635_01
Os054637_01
Os054639_01
Os054643_01
Os054645_01
Os054680_01
Os054687_01
Os054725_01
Os054737_01
Os054778_01
Os054799_01
Os054859_01
Os054861_01
Os054867_01
Os054898_01
Os054914_01
Os054922_01
Os054925_01
Os054951_01
Os054997_01
Os055037_01
Os055103_01
Os055116_01
Os055120_01
Os055147_01
Os055156_01
Os055174_01
Os055267_01
Os055322_01
Os055328_01
Os055373_01
Os055421_01
Os055470_01
Os055567_01
Os055677_01
Os055688_01
Os055991_01
Os056123_01
Os056160_01
Os056250_01
Os056254_01
Os056340_01
Os056390_01
Os056527_01
Os056528_01
Os056583_01
Os056734_01
Os056838_01
Os056928_02
Os056998_02
Os057016_02
Os057045_02
Os057063_02
Os057253_02
Os057275_02
Os057435_02
Os057480_02
Os057481_02
Os057552_02
Os057614_02

SUPPLEMENTAL TABLE 12
Oligo ID

Os000043_01
Os000193_01
Os000325_01
Os000330_01
Os000380_01
Os000382_01
Os000883_01
Os001234_01
Os001466_01
Os001488_01
Os001831_01
Os002532_01
Os002637_01
Os002827_01
Os002855_01
Os003091_01
Os003205_01
Os003276_01
Os003980_01
Os004001_01
Os004085_01
Os004668_01
Os004718_01
Os004761_01
Os004960_01
Os004990_02
Os005282_01
Os005594_01
Os005847_01
Os006291_01
Os006301_01
Os006506_01
Os006599_01
Os006743_01
Os007402_01
Os007678_02
Os008117_01
Os008372_01
Os008424_01
Os008481_01
Os008498_01
Os008648_01
Os008651_01
Os008735_01
Os008785_01
Os008796_01
Os008844_01
Os008917_01
Os008952_01
Os008955_01
Os009058_01
Os009097_01
Os009113_01
Os009209_01
Os009280_01
Os009286_01
Os009293_01
Os009348_01
Os009354_01
Os009484_01
Os009512_01
Os009581_01
Os009587_01
Os009604_01
Os009739_01
Os009786_01
Os009792_01
Os009889_01
Os009899_01
Os009909_01
Os009912_01
Os009921_01
Os009970_01
Os009989_01
Os010077_01
Os010218_01
Os010335_01
Os010342_01
Os010359_01
Os010430_01
Os010517_01
Os010583_01
Os010607_01
Os010625_01
Os010660_01
Os010668_01
Os010682_01
Os010693_01
Os010706_01
Os010749_01
Os010821_01
Os011014_01
Os011024_01
Os011039_01
Os011044_01
Os011076_01
Os011099_01
Os011149_01
Os011157_01
Os011163_01
Os011183_01
Os011306_01
Os011349_01
Os011351_01
Os011354_01
Os011356_01
Os011372_01
Os011375_01
Os011384_01
Os011385_01
Os011390_01
Os011401_01
Os011430_01
Os011460_01
Os011486_01
Os011514_01
Os011528_01
Os011534_01
Os011535_01
Os011536_01
Os011539_01
Os011544_01
Os011560_01
Os011561_01
Os011572_01
Os011577_01
Os011580_01
Os011586_01
Os011614_01
Os011661_01
Os011673_01
Os011677_01
Os011685_01
Os011686_01
Os011708_01
Os011720_01
Os011735_01
Os011752_01
Os011754_01
Os011762_01
Os011781_01
Os011790_01
Os011802_01
Os011805_01
Os011817_01
Os011848_01
Os011852_01
Os011856_01
Os011871_01
Os011874_01
Os011885_01
Os011886_01
Os011892_01
Os011920_01
Os011925_01
Os011952_01
Os011974_01
Os011976_01
Os011989_01
Os011991_01
Os011992_01
Os011996_01
Os012013_01
Os012117_01
Os012130_01
Os012151_01
Os012152_01
Os012153_01
Os012154_01
Os012177_01
Os012223_01
Os012230_01
Os012234_01
Os012237_01
Os012243_01
Os012251_01
Os012253_01
Os012254_01
Os012274_01
Os012277_01
Os012283_01
Os012286_01
Os012301_01
Os012308_01
Os012310_01
Os012311_01
Os012320_01
Os012379_01
Os012399_01
Os012411_01
Os012413_01
Os012438_01
Os012488_01
Os012503_01
Os012521_01
Os012567_01
Os012595_01
Os012616_01
Os012622_01
Os012646_01
Os012655_01
Os012660_01
Os012665_01
Os012716_01
Os012730_01
Os012731_01
Os012743_01
Os012754_01
Os012782_01
Os012843_01
Os012844_01
Os012853_01
Os012891_01
Os012893_01
Os012905_01
Os012917_01
Os012918_01
Os012936_01
Os012966_01
Os012986_01
Os012993_01
Os013008_01
Os013066_01
Os013084_01
Os013161_01
Os013180_01
Os013201_01
Os013209_01
Os013216_01
Os013224_01
Os013237_01
Os013241_01
Os013290_01
Os013303_01
Os013311_01
Os013313_01
Os013335_01
Os013345_01
Os013347_01
Os013358_01
Os013367_01
Os013389_01
Os013471_01
Os013510_01
Os013548_01
Os013560_01
Os013599_01
Os013602_01
Os013607_01
Os013645_01
Os013647_01
Os013727_01
Os013739_01
Os013817_01
Os013832_01
Os013852_01
Os013855_01
Os013920_01
Os013936_01
Os013945_01
Os013959_01
Os013985_01
Os013992_01
Os013995_01
Os014004_01
Os014027_01
Os014028_01
Os014040_01
Os014056_01
Os014071_01
Os014086_01
Os014116_01
Os014167_01
Os014176_01
Os014177_01
Os014197_01
Os014233_01
Os014237_01
Os014257_01
Os014267_01
Os014281_01
Os014300_01
Os014419_01
Os014420_01
Os014436_01
Os014491_01
Os014511_01
Os014515_01
Os014547_01
Os014548_01
Os014549_01
Os014551_01
Os014552_01
Os014564_01
Os014569_01
Os014572_01
Os014578_01
Os014598_01
Os014611_01
Os014616_01
Os014628_01
Os014629_01
Os014637_01
Os014639_01
Os014642_01
Os014663_01
Os014690_01
Os014697_01
Os014710_01
Os014714_01
Os014727_01
Os014736_01
Os014757_01
Os014758_01
Os014780_01
Os014794_01
Os014807_01
Os014824_01
Os014828_01
Os014842_01
Os014856_01
Os014859_01
Os014926_01
Os014927_01
Os014930_01
Os014948_01
Os014995_01
Os014999_01
Os015005_01
Os015008_01
Os015011_01
Os015017_01
Os015024_01
Os015036_01
Os015048_01
Os015063_01
Os015069_01
Os015075_01
Os015077_01
Os015084_01
Os015085_01
Os015089_01
Os015113_01
Os015117_01
Os015118_01
Os015122_01
Os015124_01
Os015125_01
Os015126_01
Os015128_01
Os015129_01
Os015130_01
Os015154_01
Os015195_01
Os015332_01
Os015344_01
Os015402_01
Os015438_01
Os015552_01
Os015554_01
Os015558_01
Os015559_01
Os015561_01
Os015562_01
Os015565_01
Os015569_01
Os015576_01
Os015578_01
Os015580_01
Os015584_01
Os015588_01
Os015592_01
Os015598_01
Os015601_01
Os015618_01
Os015623_01
Os015628_01
Os015639_01
Os015642_01
Os015645_01
Os015647_01
Os015649_01
Os015652_01
Os015681_01
Os015687_01
Os015696_01
Os015704_01
Os015710_01
Os015713_01
Os015715_01
Os015719_01
Os015734_01
Os015740_01
Os015742_01
Os015746_01
Os015757_01
Os015768_01
Os015781_01
Os015786_01
Os015788_01
Os015789_01
Os015791_01
Os015802_01
Os015804_01
Os015806_01
Os015810_01
Os015814_01
Os015817_01
Os015828_01
Os015829_01
Os015830_01
Os015838_01
Os015843_01
Os015846_01
Os015862_01
Os015864_01
Os015871_01
Os015872_01
Os015874_01
Os015882_01
Os015885_01
Os015907_01
Os015916_01
Os015936_01
Os015937_01
Os015955_01
Os015961_01
Os015974_01
Os015976_01
Os015979_01
Os015986_01
Os015989_01
Os016001_01
Os016003_01
Os016005_01
Os016010_01
Os016013_01
Os016024_01
Os016028_01
Os016032_01
Os016034_01
Os016039_01
Os016045_01
Os016047_01
Os016057_01
Os016081_01
Os016087_01
Os016089_01
Os016096_01
Os016098_01
Os016104_01
Os016107_01
Os016109_01
Os016111_01
Os016128_01
Os016137_01
Os016150_01
Os016153_01
Os016171_01
Os016172_01
Os016189_01
Os016194_01
Os016200_01
Os016205_01
Os016223_01
Os016233_01
Os016234_01
Os016254_01
Os016255_01
Os016256_01
Os016259_01
Os016262_01
Os016269_01
Os016277_01
Os016292_01
Os016297_01
Os016298_01
Os016303_01
Os016317_01
Os016318_01
Os016320_01
Os016328_01
Os016335_01
Os016344_01
Os016352_01
Os016353_01
Os016361_01
Os016374_01
Os016397_01
Os016400_01
Os016401_01
Os016402_01
Os016404_01
Os016405_01
Os016406_01
Os016408_01
Os016409_01
Os016410_01
Os016411_01
Os016419_01
Os016424_01
Os016451_01
Os016453_01
Os016462_01
Os016463_01
Os016466_01
Os016468_01
Os016478_01
Os016480_01
Os016496_01
Os016498_01
Os016507_01
Os016518_01
Os016528_01
Os016533_01
Os016551_01
Os016555_01
Os016557_01
Os016558_01
Os016577_01
Os016585_01
Os016588_01
Os016589_01
Os016593_01
Os016597_01
Os016600_01
Os016619_01
Os016622_01
Os016625_01
Os016633_01
Os016636_01
Os016642_01
Os016648_01
Os016664_01
Os016680_01
Os016684_01
Os016692_01
Os016693_01
Os016694_01
Os016695_01
Os016702_01
Os016703_01
Os016730_01
Os016741_01
Os016787_01
Os016790_01
Os016874_01
Os016911_01
Os016916_01
Os016922_01
Os016925_01
Os016927_01
Os016932_01
Os016933_01
Os016934_01
Os016937_01
Os016959_01
Os016962_01
Os016965_01
Os016971_01
Os016972_01
Os016986_01
Os016991_01
Os017006_01
Os017007_01
Os017017_01
Os017028_01
Os017030_01
Os017059_01
Os017060_01
Os017076_01
Os017086_01
Os017090_01
Os017103_01
Os017108_01
Os017109_01
Os017110_01
Os017120_01
Os017126_01
Os017127_01
Os017129_01
Os017134_01
Os017136_01
Os017141_01
Os017146_01
Os017166_01
Os017173_01
Os017176_01
Os017185_01
Os017187_01
Os017226_01
Os017228_01
Os017232_01
Os017246_01
Os017253_01
Os017254_01
Os017257_01
Os017272_01
Os017287_01
Os017289_01
Os017291_01
Os017304_01
Os017310_01
Os017315_01
Os017316_01
Os017317_01
Os017325_01
Os017343_01
Os017361_01
Os017372_01
Os017377_01
Os017381_01
Os017390_01
Os017399_01
Os017401_01
Os017412_01
Os017416_01
Os017432_01
Os017433_01
Os017441_01
Os017452_01
Os017458_01
Os017467_01
Os017468_01
Os017476_01
Os017477_01
Os017485_01
Os017488_01
Os017505_01
Os017507_01
Os017524_01
Os017530_01
Os017539_01
Os017542_01
Os017544_01
Os017553_01
Os017605_01
Os017635_01
Os017655_01
Os017671_01
Os017697_01
Os017698_01
Os017709_01
Os017722_01
Os017753_01
Os017761_01
Os017762_01
Os017791_01
Os017796_01
Os017808_01
Os017828_01
Os017829_01
Os017848_01
Os017855_01
Os017858_01
Os017874_01
Os017876_01
Os017880_01
Os017881_01
Os017891_01
Os017908_01
Os017912_01
Os017914_01
Os017917_01
Os017929_01
Os017931_01
Os017949_01
Os017956_01
Os017957_01
Os017998_01
Os018002_01
Os018023_01
Os018028_01
Os018045_01
Os018057_01
Os018068_01
Os018073_01
Os018089_01
Os018090_01
Os018092_01
Os018099_01
Os018103_01
Os018106_01
Os018108_01
Os018113_01
Os018117_01
Os018122_01
Os018130_01
Os018131_01
Os018135_01
Os018137_01
Os018140_01
Os018148_01
Os018151_01
Os018155_01
Os018158_01
Os018164_01
Os018166_01
Os018168_01
Os018169_01
Os018174_01
Os018209_01
Os018225_01
Os018232_01
Os018234_01
Os018239_01
Os018240_01
Os018247_01
Os018256_01
Os018267_01
Os018269_01
Os018298_01
Os018304_01
Os018335_01
Os018360_01
Os018361_01
Os018367_01
Os018375_01
Os018422_01
Os018436_01
Os018447_01
Os018473_01
Os018484_01
Os018499_01
Os018519_01
Os018543_01
Os018560_01
Os018562_01
Os018589_01
Os018631_01
Os018647_01
Os018651_01
Os018662_01
Os018679_01
Os018708_01
Os018716_01
Os018726_01
Os018730_01
Os018734_01
Os018735_01
Os018743_01
Os018747_01
Os018756_01
Os018771_01
Os018776_01
Os018782_01
Os018785_01
Os018806_01
Os018809_01
Os018810_01
Os018826_01
Os018829_01
Os018834_01
Os018841_01
Os018847_01
Os018854_01
Os018856_01
Os018857_01
Os018867_01
Os018868_01
Os018874_01
Os018877_01
Os018882_01
Os018883_01
Os018887_01
Os018896_01
Os018900_01
Os018901_01
Os018907_01
Os018908_01
Os018910_01
Os018917_01
Os018926_01
Os018931_01
Os018955_01
Os018980_01
Os018990_01
Os018992_01
Os018993_01
Os018994_01
Os019008_01
Os019012_01
Os019013_01
Os019020_01
Os019021_01
Os019041_01
Os019042_01
Os019051_01
Os019053_01
Os019059_01
Os019076_01
Os019080_01
Os019085_01
Os019095_01
Os019103_01
Os019117_01
Os019121_01
Os019126_01
Os019134_01
Os019144_01
Os019145_01
Os019160_01
Os019165_01
Os019171_01
Os019175_01
Os019176_01
Os019177_01
Os019182_01
Os019183_01
Os019191_01
Os019192_01
Os019207_01
Os019208_01
Os019217_01
Os019218_01
Os019219_01
Os019222_01
Os019234_01
Os019235_01
Os019268_01
Os019281_01
Os019282_01
Os019300_01
Os019302_01
Os019305_01
Os019306_01
Os019307_01
Os019317_01
Os019332_01
Os019334_01
Os019336_01
Os019365_01
Os019373_01
Os019381_01
Os019382_01
Os019386_01
Os019391_01
Os019399_01
Os019401_01
Os019405_01
Os019409_01
Os019410_01
Os019412_01
Os019419_01
Os019420_01
Os019421_01
Os019422_01
Os019423_01
Os019436_01
Os019475_01
Os019476_01
Os019489_01
Os019493_01
Os019495_01
Os019499_01
Os019501_01
Os019504_01
Os019514_01
Os019527_01
Os019530_01
Os019533_01
Os019535_01
Os019541_01
Os019555_01
Os019574_01
Os019585_01
Os019649_01
Os019650_01
Os019653_01
Os019655_01
Os019656_01
Os019659_01
Os019663_01
Os019705_01
Os019712_01
Os019715_01
Os019726_01
Os019741_01
Os019750_01
Os019752_01
Os019755_01
Os019786_01
Os019789_01
Os019792_01
Os019796_01
Os019798_01
Os019800_01
Os019803_01
Os019842_01
Os019852_01
Os019856_01
Os019878_01
Os019884_01
Os019899_01
Os019909_01
Os019913_01
Os019957_01
Os019977_01
Os019985_01
Os020009_01
Os020034_01
Os020111_01
Os020143_01
Os020148_01
Os020149_01
Os020152_01
Os020156_01
Os020160_01
Os020224_01
Os020225_01
Os020227_01
Os020243_01
Os020253_01
Os020285_01
Os020289_01
Os020298_01
Os020299_01
Os020301_01
Os020309_01
Os020322_01
Os020324_01
Os020329_01
Os020372_01
Os020394_01
Os020401_01
Os020423_01
Os020425_01
Os020448_01
Os020451_01
Os020455_01
Os020456_01
Os020466_01
Os020480_01
Os020503_01
Os020509_01
Os020544_01
Os020556_01
Os020566_01
Os020572_01
Os020588_01
Os020592_01
Os020595_01
Os020603_01
Os020625_01
Os020629_01
Os020640_01
Os020642_01
Os020651_01
Os020674_01
Os020677_01
Os020684_01
Os020690_01
Os020699_01
Os020704_01
Os020708_01
Os020712_01
Os020722_01
Os020753_01
Os020763_01
Os020766_01
Os020776_01
Os020795_01
Os020798_01
Os020802_01
Os020819_01
Os020842_01
Os020846_01
Os020848_01
Os020851_01
Os020861_01
Os020867_01
Os020885_01
Os020897_01
Os020899_01
Os020905_01
Os020918_01
Os020926_01
Os020933_01
Os020937_01
Os020938_01
Os020970_01
Os020978_01
Os020980_01
Os020987_01
Os020988_01
Os020995_01
Os021007_01
Os021012_01
Os021017_01
Os021022_01
Os021030_01
Os021034_01
Os021036_01
Os021042_01
Os021048_01
Os021050_01
Os021053_01
Os021062_01
Os021063_01
Os021065_01
Os021067_01
Os021079_01
Os021093_01
Os021094_01
Os021099_01
Os021110_01
Os021112_01
Os021114_01
Os021125_01
Os021128_01
Os021135_01
Os021137_01
Os021161_01
Os021163_01
Os021193_01
Os021196_01
Os021205_01
Os021209_01
Os021213_01
Os021258_01
Os021260_01
Os021270_01
Os021282_01
Os021291_01
Os021305_01
Os021309_01
Os021320_01
Os021321_01
Os021326_01
Os021334_01
Os021362_01
Os021385_01
Os021393_01
Os021412_01
Os021422_01
Os021438_01
Os021440_01
Os021457_01
Os021458_01
Os021518_01
Os021521_01
Os021525_01
Os021595_01
Os021606_01
Os021609_01
Os021615_01
Os021616_01
Os021657_01
Os021670_01
Os021671_01
Os021693_01
Os021730_01
Os021743_01
Os021744_01
Os021747_01
Os021783_01
Os021793_01
Os021803_01
Os021804_01
Os021811_01
Os021817_01
Os021819_01
Os021825_01
Os021833_01
Os021844_01
Os021859_01
Os021880_01
Os021888_01
Os021892_01
Os021934_01
Os021938_01
Os021956_01
Os021960_01
Os021969_01
Os021985_01
Os021995_01
Os022020_01
Os022021_01
Os022022_01
Os022040_01
Os022048_01
Os022052_01
Os022064_01
Os022066_01
Os022067_01
Os022077_01
Os022079_01
Os022096_01
Os022115_01
Os022118_01
Os022135_01
Os022163_01
Os022186_01
Os022188_01
Os022191_01
Os022194_01
Os022202_01
Os022214_01
Os022255_01
Os022259_01
Os022267_01
Os022312_01
Os022342_01
Os022354_01
Os022363_01
Os022365_01
Os022378_01
Os022424_01
Os022429_01
Os022447_01
Os022455_01
Os022460_01
Os022465_01
Os022498_01
Os022503_01
Os022506_01
Os022520_01
Os022527_01
Os022547_01
Os022593_01
Os022615_01
Os022622_01
Os022624_01
Os022636_01
Os022644_01
Os022650_01
Os022665_01
Os022675_01
Os022685_01
Os022694_01
Os022711_01
Os022742_01
Os022745_01
Os022750_01
Os022753_01
Os022762_01
Os022763_01
Os022768_01
Os022783_01
Os022795_01
Os022803_01
Os022808_01
Os022812_01
Os022826_01
Os022831_01
Os022836_01
Os022838_01
Os022843_01
Os022850_01
Os022862_01
Os022869_01
Os022873_01
Os022885_01
Os022890_01
Os022894_01
Os022897_01
Os022899_01
Os022918_01
Os022920_01
Os022979_01
Os022988_01
Os023021_01
Os023128_01
Os023143_01
Os023170_01
Os023177_01
Os023199_01
Os023210_01
Os023228_01
Os023263_01
Os023274_01
Os023277_01
Os023279_01
Os023280_01
Os023391_01
Os023393_01
Os023404_01
Os023426_01
Os023432_01
Os023443_01
Os023474_01
Os023528_01
Os023541_01
Os023549_01
Os023565_01
Os023581_01
Os023592_01
Os023606_01
Os023665_01
Os023674_01
Os023707_01
Os023771_01
Os023876_01
Os023955_01
Os023968_01
Os023973_01
Os024002_01
Os024017_01
Os024025_01
Os024077_01
Os024119_01
Os024149_01
Os024150_01
Os024172_01
Os024187_01
Os024201_01
Os024214_01
Os024249_01
Os024263_01
Os024269_01
Os024300_01
Os024326_01
Os024344_01
Os024349_01
Os024364_01
Os024429_01
Os024478_01
Os024565_01
Os024588_01
Os024591_01
Os024595_01
Os024621_01
Os024622_01
Os024627_01
Os024638_01
Os024643_01
Os024645_01
Os024660_01
Os024665_01
Os024670_01
Os024671_01
Os024683_01
Os024716_01
Os024727_01
Os024728_01
Os024740_01
Os024770_01
Os024772_01
Os024779_01
Os024787_01
Os024795_01
Os024803_01
Os024806_01
Os024815_01
Os024820_01
Os024824_01
Os024826_01
Os024833_01
Os024845_01
Os024851_01
Os024863_01
Os024868_01
Os024869_01
Os024878_01
Os024879_01
Os024881_01
Os024885_01
Os024897_01
Os024901_01
Os024920_01
Os024932_01
Os024962_01
Os024971_01
Os024981_01
Os024990_01
Os024992_01
Os024993_01
Os025001_01
Os025026_01
Os025035_01
Os025042_01
Os025054_01
Os025075_01
Os025076_01
Os025079_01
Os025086_01
Os025102_01
Os025106_01
Os025109_01
Os025127_01
Os025147_01
Os025150_01
Os025155_01
Os025165_01
Os025180_01
Os025181_01
Os025191_01
Os025193_01
Os025200_01
Os025216_01
Os025244_01
Os025249_01
Os025297_01
Os025321_01
Os025343_01
Os025363_01
Os025373_01
Os025383_01
Os025509_01
Os025532_01
Os025611_01
Os025681_01
Os025779_01
Os025792_01
Os025793_01
Os025802_01
Os025822_01
Os025847_01
Os025860_01
Os025882_01
Os025886_01
Os025893_01
Os025895_01
Os025896_01
Os025899_01
Os025930_01
Os025939_01
Os025968_01
Os025976_01
Os025984_01
Os025985_01
Os025990_01
Os026009_01
Os026043_01
Os026051_01
Os026066_01
Os026084_01
Os026090_01
Os026092_01
Os026111_01
Os026121_01
Os026127_01
Os026131_01
Os026136_01
Os026137_01
Os026142_01
Os026148_01
Os026156_01
Os026164_01
Os026172_01
Os026176_01
Os026180_01
Os026186_01
Os026200_01
Os026225_01
Os026252_01
Os026296_01
Os026308_01
Os026316_01
Os026336_01
Os026397_01
Os026436_01
Os026451_01
Os026457_01
Os026461_01
Os026495_01
Os026505_01
Os026608_01
Os026615_01
Os026653_01
Os026663_01
Os026690_01
Os026736_01
Os026761_01
Os026763_01
Os026896_01
Os026917_01
Os026920_01
Os027006_01
Os027044_01
Os027200_01
Os027221_01
Os027298_01
Os027369_01
Os027455_01
Os027484_01
Os027533_01
Os027555_01
Os027575_01
Os027591_01
Os027621_01
Os027623_01
Os027639_01
Os027640_01
Os027663_01
Os027683_01
Os027695_01
Os027698_01
Os027704_01
Os027720_01
Os027736_01
Os027758_01
Os027773_01
Os027793_01
Os027799_01
Os027849_01
Os027856_01
Os027864_01
Os027872_01
Os027875_01
Os027883_01
Os027907_01
Os027919_01
Os027940_01
Os027944_01
Os027957_01
Os027963_01
Os027973_01
Os027981_01
Os028000_01
Os028021_01
Os028044_01
Os028051_01
Os028055_01
Os028085_01
Os028086_01
Os028101_01
Os028102_01
Os028123_01
Os028128_01
Os028131_01
Os028143_01
Os028147_01
Os028149_01
Os028194_01
Os028218_01
Os028226_01
Os028235_01
Os028247_01
Os028257_01
Os028269_01
Os028284_01
Os028314_01
Os028316_01
Os028323_01
Os028326_01
Os028345_01
Os028358_01
Os028371_01
Os028374_01
Os028375_01
Os028400_01
Os028407_01
Os028424_01
Os028440_01
Os028442_01
Os028443_01
Os028460_01
Os028467_01
Os028485_01
Os028511_01
Os028513_01
Os028532_01
Os028538_01
Os028566_01
Os028567_01
Os028583_01
Os028597_01
Os028599_01
Os028601_01
Os028607_01
Os028616_01
Os028621_01
Os028632_01
Os028652_01
Os028653_01
Os028654_01
Os028666_01
Os028669_01
Os028685_01
Os028699_01
Os028700_01
Os028703_01
Os028721_01
Os028745_01
Os028755_01
Os028756_01
Os028757_01
Os028767_01
Os028792_01
Os028793_01
Os028796_01
Os028820_01
Os028860_01
Os028861_01
Os028863_01
Os028886_01
Os028891_01
Os028906_01
Os028914_01
Os028929_01
Os028931_01
Os028944_01
Os028952_01
Os028953_01
Os028957_01
Os029002_01
Os029012_01
Os029017_01
Os029018_01
Os029091_01
Os029094_01
Os029100_01
Os029101_01
Os029112_01
Os029125_01
Os029148_01
Os029217_01
Os029220_01
Os029306_01
Os029465_01
Os029556_01
Os029665_01
Os029713_01
Os029902_01
Os030125_01
Os030165_01
Os030197_01
Os030274_01
Os030370_01
Os030471_01
Os030489_01
Os030507_01
Os030553_01
Os030602_01
Os030632_01
Os030681_01
Os030685_01
Os030702_01
Os030719_01
Os030750_01
Os030807_01
Os030808_01
Os030873_01
Os030882_01
Os030963_01
Os031025_01
Os031031_01
Os031206_01
Os031214_01
Os031377_01
Os031452_01
Os031464_01
Os031496_01
Os031528_01
Os031529_01
Os031532_01
Os031567_01
Os031652_01
Os031659_01
Os031666_01
Os031747_01
Os031750_01
Os031755_01
Os031800_01
Os031832_01
Os031879_01
Os031901_01
Os031934_01
Os031977_01
Os032054_01
Os032064_01
Os032118_01
Os032123_01
Os032150_01
Os032169_01
Os032231_01
Os032245_01
Os032269_01
Os032295_01
Os032388_01
Os032481_01
Os032590_01
Os032657_01
Os032705_01
Os032707_01
Os032844_01
Os032872_01
Os033219_01
Os033248_01
Os033292_01
Os033328_01
Os033512_01
Os033524_01
Os033533_01
Os033612_01
Os033686_01
Os033694_01
Os033700_01
Os033703_01
Os033818_01
Os034056_01
Os034141_01
Os034157_01
Os034230_01
Os034275_01
Os034305_01
Os034329_01
Os034336_01
Os034411_01
Os034449_01
Os034467_01
Os034473_01
Os034581_01
Os034601_01
Os034692_01
Os034737_01
Os034765_01
Os034848_01
Os034904_01
Os035035_01
Os035070_01
Os035091_01
Os035143_01
Os035159_01
Os035242_01
Os035261_01
Os035336_01
Os035373_01
Os035375_01
Os035494_01
Os035514_01
Os035712_01
Os035785_01
Os035822_01
Os035865_01
Os035879_01
Os035901_01
Os036044_01
Os036114_01
Os036254_01
Os036329_01
Os036334_01
Os036343_01
Os036428_01
Os036435_01
Os036476_01
Os036484_01
Os036570_01
Os036612_01
Os036625_01
Os036634_01
Os036713_01
Os036719_01
Os036731_01
Os036804_01
Os036816_01
Os036818_01
Os036933_01
Os037062_01
Os037118_01
Os037234_01
Os037451_01
Os037490_01
Os037497_01
Os037533_01
Os037639_01
Os037704_01
Os037720_01
Os037742_01
Os037751_01
Os037801_01
Os037827_01
Os037986_01
Os037993_01
Os038027_01
Os038114_01
Os038123_01
Os038267_01
Os038269_01
Os038282_01
Os038309_01
Os038316_01
Os038340_01
Os038372_01
Os038384_01
Os038429_01
Os038486_01
Os038499_01
Os038546_01
Os038553_01
Os038561_01
Os038630_01
Os038648_01
Os038705_01
Os038790_01
Os038816_01
Os038843_01
Os038869_01
Os038976_01
Os039171_01
Os039180_01
Os039348_01
Os039361_01
Os039375_01
Os039391_01
Os039422_01
Os039566_01
Os039597_01
Os039833_01
Os039856_01
Os039865_01
Os039889_01
Os039905_01
Os039914_01
Os040126_01
Os040142_01
Os040158_01
Os040169_01
Os040172_01
Os040182_01
Os040199_01
Os040260_01
Os040261_01
Os040272_01
Os040340_01
Os040509_01
Os040581_01
Os040671_01
Os040673_01
Os040684_01
Os040695_01
Os040718_01
Os040721_01
Os040884_01
Os040906_01
Os040985_01
Os040994_01
Os041019_01
Os041219_01
Os041320_01
Os041327_01
Os041350_01
Os041428_01
Os041435_01
Os041452_01
Os041455_01
Os041510_01
Os041534_01
Os041545_01
Os041562_01
Os041580_01
Os041745_01
Os041755_01
Os041920_01
Os041926_01
Os041946_01
Os042068_01
Os042194_01
Os042253_01
Os042321_01
Os042566_01
Os042568_01
Os042717_01
Os042787_01
Os042919_01
Os042940_01
Os042954_01
Os042993_01
Os042998_01
Os043124_01
Os043187_01
Os043838_01
Os043879_01
Os043894_01
Os043934_01
Os043995_01
Os044218_01
Os044223_01
Os044249_01
Os044265_01
Os044274_01
Os044451_01
Os044453_01
Os044464_01
Os044469_01
Os044470_01
Os044492_01
Os044500_01
Os044570_01
Os044583_01
Os044590_01
Os044638_01
Os044712_01
Os044742_01
Os044796_01
Os044826_01
Os044844_01
Os044856_01
Os044916_01
Os044964_01
Os045017_01
Os045025_01
Os045135_01
Os045174_01
Os045176_01
Os045267_01
Os045282_01
Os045483_01
Os045500_01
Os045510_01
Os045512_01
Os045519_01
Os045559_01
Os045640_01
Os045670_01
Os045679_01
Os045857_01
Os045905_01
Os045934_01
Os045984_01
Os046001_01
Os046191_01
Os046228_01
Os046328_01
Os046422_01
Os046505_01
Os046538_01
Os046563_01
Os046580_01
Os046691_01
Os046721_01
Os046736_01
Os046780_01
Os046788_01
Os046811_01
Os046813_01
Os046944_01
Os047109_01
Os047200_01
Os047234_01
Os047235_01
Os047297_01
Os047299_01
Os047345_01
Os047382_01
Os047400_01
Os047403_01
Os047431_01
Os047518_01
Os047519_01
Os047520_01
Os047524_01
Os047527_01
Os047551_01
Os047559_01
Os047560_01
Os047566_01
Os047626_01
Os047685_01
Os047974_01
Os047981_01
Os048061_01
Os048089_01
Os048165_01
Os048228_01
Os048230_01
Os048270_01
Os048375_01
Os048518_01
Os048532_01
Os048534_01
Os048586_01
Os048668_01
Os048734_01
Os048743_01
Os048752_01
Os048771_01
Os048930_01
Os049028_01
Os049079_01
Os049207_01
Os049375_01
Os049476_01
Os049676_01
Os049787_01
Os049821_01
Os049881_01
Os049908_01
Os050004_01
Os050080_01
Os050085_01
Os050109_01
Os050163_01
Os050238_01
Os050242_01
Os050347_01
Os050348_01
Os050470_01
Os050558_01
Os050616_01
Os050647_01
Os050770_01
Os050830_01
Os050864_01
Os050909_01
Os050910_01
Os050912_01
Os051017_01
Os051120_01
Os051156_01
Os051214_01
Os051684_01
Os051704_01
Os051725_01
Os051811_01
Os051829_01
Os051868_01
Os051904_01
Os051933_01
Os051957_01
Os051965_01
Os051971_01
Os051973_01
Os052009_01
Os052023_01
Os052024_01
Os052043_01
Os052046_01
Os052047_01
Os052048_01
Os052049_01
Os052052_01
Os052084_01
Os052086_01
Os052088_01
Os052107_01
Os052114_01
Os052123_01
Os052124_01
Os052125_01
Os052130_01
Os052151_01
Os052205_01
Os052217_01
Os052218_01
Os052233_01
Os052234_01
Os052242_01
Os052244_01
Os052254_01
Os052268_01
Os052271_01
Os052272_01
Os052274_01
Os052316_01
Os052319_01
Os052328_01
Os052341_01
Os052348_01
Os052350_01
Os052356_01
Os052384_01
Os052387_01
Os052395_01
Os052404_01
Os052418_01
Os052419_01
Os052425_01
Os052431_01
Os052471_01
Os052508_01
Os052512_01
Os052537_01
Os052580_01
Os052620_01
Os052632_01
Os052643_01
Os052646_01
Os052648_01
Os052653_01
Os052655_01
Os052673_01
Os052684_01
Os052688_01
Os052705_01
Os052714_01
Os052715_01
Os052724_01
Os052747_01
Os052772_01
Os052776_01
Os052788_01
Os052791_01
Os052826_01
Os052831_01
Os052849_01
Os052860_01
Os052872_01
Os052876_01
Os052905_01
Os052933_01
Os052936_01
Os052982_01
Os052985_01
Os053072_01
Os053076_01
Os053093_01
Os053097_01
Os053101_01
Os053105_01
Os053107_01
Os053111_01
Os053115_01
Os053135_01
Os053144_01
Os053173_01
Os053175_01
Os053186_01
Os053189_01
Os053205_01
Os053218_01
Os053224_01
Os053240_01
Os053241_01
Os053266_01
Os053281_01
Os053301_01
Os053321_01
Os053363_01
Os053368_01
Os053372_01
Os053419_01
Os053442_01
Os053447_01
Os053460_01
Os053478_01
Os053484_01
Os053504_01
Os053518_01
Os053523_01
Os053524_01
Os053534_01
Os053557_01
Os053569_01
Os053583_01
Os053591_01
Os053594_01
Os053595_01
Os053603_01
Os053623_01
Os053642_01
Os053668_01
Os053684_01
Os053705_01
Os053716_01
Os053779_01
Os053815_01
Os053816_01
Os053828_01
Os053829_01
Os053840_01
Os053846_01
Os053861_01
Os053906_01
Os053924_01
Os053931_01
Os053941_01
Os053971_01
Os054022_01
Os054031_01
Os054072_01
Os054176_02
Os054193_01
Os054202_01
Os054208_01
Os054225_01
Os054234_01
Os054238_01
Os054245_01
Os054268_01
Os054273_01
Os054335_01
Os054382_01
Os054383_01
Os054416_01
Os054432_01
Os054456_01
Os054471_01
Os054483_01
Os054495_01
Os054499_01
Os054509_01
Os054527_01
Os054542_01
Os054556_01
Os054558_01
Os054577_01
Os054581_01
Os054588_01
Os054607_01
Os054619_01
Os054625_01
Os054653_01
Os054656_01
Os054707_01
Os054723_01
Os054726_01
Os054728_01
Os054752_01
Os054768_01
Os054774_01
Os054779_01
Os054791_01
Os054817_01
Os054819_01
Os054820_01
Os054829_01
Os054836_01
Os054842_01
Os054857_01
Os054862_02
Os054869_01
Os054878_01
Os054888_01
Os054900_01
Os054903_01
Os054907_01
Os054910_01
Os054921_01
Os054932_01
Os054936_01
Os054955_01
Os054961_01
Os054964_01
Os054975_01
Os054979_01
Os055002_01
Os055005_01
Os055011_01
Os055017_01
Os055025_01
Os055048_01
Os055050_01
Os055067_01
Os055096_01
Os055136_01
Os055139_01
Os055140_01
Os055154_01
Os055167_01
Os055169_02
Os055184_01
Os055187_01
Os055191_01
Os055218_01
Os055241_01
Os055245_01
Os055250_01
Os055252_01
Os055256_01
Os055261_01
Os055263_01
Os055292_01
Os055307_01
Os055324_01
Os055333_01
Os055337_01
Os055351_01
Os055352_01
Os055361_01
Os055387_01
Os055391_01
Os055401_01
Os055408_01
Os055427_01
Os055447_01
Os055457_01
Os055458_01
Os055461_01
Os055487_01
Os055498_01
Os055510_01
Os055513_01
Os055515_01
Os055539_01
Os055545_01
Os055571_01
Os055579_01
Os055589_01
Os055603_01
Os055616_01
Os055621_01
Os055635_01
Os055651_01
Os055656_01
Os055681_01
Os055715_01
Os055720_01
Os055722_01
Os055788_01
Os055813_01
Os055814_01
Os055860_01
Os055947_01
Os055966_01
Os055968_01
Os055982_01
Os056004_01
Os056010_01
Os056047_01
Os056092_01
Os056110_01
Os056131_01
Os056162_01
Os056170_01
Os056174_01
Os056193_01
Os056213_01
Os056219_01
Os056223_01
Os056266_01
Os056277_01
Os056288_01
Os056317_01
Os056330_01
Os056331_01
Os056368_01
Os056372_01
Os056387_01
Os056391_01
Os056392_01
Os056419_01
Os056424_01
Os056427_01
Os056441_01
Os056454_01
Os056473_01
Os056481_01
Os056492_01
Os056518_01
Os056562_01
Os056572_01
Os056574_01
Os056622_02
Os056624_01
Os056644_01
Os056651_01
Os056663_01
Os056686_01
Os056703_01
Os056714_01
Os056722_01
Os056750_01
Os056752_01
Os056768_01
Os056780_01
Os056781_01
Os056785_01
Os056788_01
Os056800_01
Os056802_01
Os056809_01
Os056811_01
Os056816_01
Os056821_01
Os056826_01
Os056844_01
Os056851_01
Os056852_01
Os056866_01
Os056869_01
Os056870_01
Os056872_01
Os056919_02
Os056936_01
Os056974_02
Os057029_01
Os057092_02
Os057111_02
Os057118_02
Os057153_02
Os057165_02
Os057176_02
Os057204_01
Os057225_02
Os057257_02
Os057295_02
Os057318_02
Os057451_02
Os057454_02
Os057492_02
Os057577_02
Os057666_02

SUPPLEMENTAL TABLE 13
Os000062_01
Os000085_01
Os000102_01
Os000152_01
Os000160_01
Os000187_01
Os000192_01
Os000237_01
Os000254_01
Os000262_01
Os000266_01
Os000300_01
Os000304_01
Os000326_01
Os000380_01
Os000386_01
Os000561_01
Os000629_01
Os000646_01
Os000669_01
Os000689_01
Os000831_01
Os000836_01
Os000890_01
Os000897_01
Os000979_01
Os000994_01
Os001002_01
Os001008_01
Os001035_01
Os001242_01
Os001260_01
Os001385_01
Os001458_01
Os001529_01
Os013976_01
Os001579_01
Os001610_01
Os001637_01
Os001649_01
Os001813_01
Os001829_01
Os001838_01
Os001842_01
Os001848_01
Os001896_01
Os001903_01
Os001993_01
Os002013_01
Os002040_01
Os002092_01
Os002181_01
Os002531_01
Os002648_01
Os002673_01
Os002679_01
Os002723_01
Os002790_01
Os002827_01
Os003016_01
Os003025_01
Os003800_01
Os004012_01
Os004097_01
Os004235_01
Os004287_01
Os004298_01
Os004301_01
Os004517_01
Os004748_01
Os004826_01
Os015666_01
Os004963_01
Os004990_02
Os005941_01
Os005956_01
Os006041_01
Os006506_01
Os006635_01
Os006733_01
Os006969_01
Os007493_01
Os007764_01
Os007891_01
Os007934_01
Os008192_01
Os008286_01
Os008325_02
Os008398_01
Os008400_01
Os008420_01
Os008437_01
Os008538_01
Os008558_01
Os008602_01
Os008615_01
Os008639_01
Os008640_01
Os008668_01
Os008690_01
Os008721_01
Os008722_01
Os008754_01
Os008787_01
Os008796_01
Os008842_01
Os008878_01
Os016868_01
Os008886_01
Os008955_01
Os008962_01
Os008968_01
Os008970_01
Os008977_01
Os008979_01
Os008985_01
Os009025_01
Os009045_01
Os009091_01
Os009099_01
Os009102_01
Os009110_01
Os009116_01
Os009117_01
Os009138_01
Os009140_01
Os009190_01
Os009214_01
Os009232_01
Os009237_01
Os009268_01
Os009278_01
Os009289_01
Os009291_01
Os009308_01
Os009323_01
Os009340_01
Os009362_01
Os009506_01
Os009547_01
Os009549_01
Os009556_01
Os009577_01
Os018106_01
Os009582_01
Os009637_01
Os009657_01
Os009685_01
Os009711_01
Os009744_01
Os009749_01
Os009808_01
Os009822_01
Os009836_01
Os009847_01
Os009849_01
Os009858_01
Os009863_01
Os009883_01
Os009889_01
Os009904_01
Os009933_01
Os009986_01
Os010052_01
Os010090_01
Os010107_01
Os010109_01
Os010113_01
Os010115_01
Os010172_01
Os010192_01
Os010276_01
Os010299_01
Os010372_01
Os010376_01
Os010390_01
Os010407_01
Os010412_01
Os010587_01
Os018818_01
Os010727_01
Os010843_01
Os010894_01
Os010918_01
Os010946_01
Os011150_01
Os011386_01
Os011429_01
Os011537_01
Os011544_01
Os011576_01
Os011578_01
Os011617_01
Os011642_01
Os011660_01
Os011662_01
Os011690_01
Os011734_01
Os011738_01
Os011748_01
Os011760_01
Os011793_01
Os011812_01
Os011816_01
Os011872_01
Os011976_01
Os011979_01
Os011998_01
Os012042_01
Os012099_01
Os012117_01
Os012119_01
Os012123_01
Os012184_01
Os012188_01
Os020616_01
Os012251_01
Os012290_01
Os012292_01
Os012334_01
Os012363_01
Os012373_01
Os012410_01
Os012417_01
Os012466_01
Os012487_01
Os012505_01
Os012543_01
Os012571_01
Os012572_01
Os012575_01
Os012588_01
Os012603_01
Os012617_01
Os012633_01
Os012641_01
Os012643_01
Os012664_01
Os012667_01
Os012714_01
Os012775_01
Os012807_01
Os012844_01
Os012876_01
Os012878_01
Os012921_01
Os012926_01
Os012960_01
Os012966_01
Os013014_01
Os013028_01
Os021978_01
Os013043_01
Os013058_01
Os013059_01
Os013123_01
Os013156_01
Os013159_01
Os013169_01
Os013184_01
Os013207_01
Os013216_01
Os013226_01
Os013246_01
Os013289_01
Os013295_01
Os013309_01
Os013340_01
Os013388_01
Os013389_01
Os013394_01
Os013398_01
Os013497_01
Os013518_01
Os013551_01
Os013591_01
Os013613_01
Os013616_01
Os013692_01
Os013744_01
Os013803_01
Os013857_01
Os013918_01
Os013937_01
Os013939_01
Os013941_01
Os013945_01
Os026758_01
Os014001_01
Os014039_01
Os014108_01
Os014118_01
Os014169_01
Os014187_01
Os014194_01
Os014203_01
Os014273_01
Os014353_01
Os014373_01
Os014378_01
Os014383_01
Os014453_01
Os014470_01
Os014473_01
Os014505_01
Os014548_01
Os014692_01
Os014707_01
Os014717_01
Os014849_01
Os014863_01
Os014912_01
Os014913_01
Os015005_01
Os015044_01
Os015101_01
Os015156_01
Os015340_01
Os015534_01
Os015547_01
Os015565_01
Os015589_01
Os015654_01
Os031525_01
Os031560_01
Os031566_01
Os015673_01
Os015708_01
Os015742_01
Os015745_01
Os015807_01
Os015868_01
Os015881_01
Os015882_01
Os016102_01
Os016171_01
Os016179_01
Os016194_01
Os016205_01
Os016218_01
Os016221_01
Os016227_01
Os016284_01
Os016297_01
Os016309_01
Os016335_01
Os016356_01
Os016476_01
Os016484_01
Os016537_01
Os016540_01
Os016615_01
Os016616_01
Os016617_01
Os016642_01
Os016716_01
Os016756_01
Os016769_01
Os016816_01
Os016845_01
Os016851_01
Os036995_01
Os037155_01
Os037326_01
Os016878_01
Os016882_01
Os016925_01
Os016931_01
Os017002_01
Os017044_01
Os017087_01
Os017092_01
Os017099_01
Os017149_01
Os017258_01
Os017289_01
Os017376_01
Os017386_01
Os017398_01
Os017399_01
Os017400_01
Os017409_01
Os017465_01
Os017521_01
Os017559_01
Os017601_01
Os017628_01
Os017640_01
Os017680_01
Os017686_01
Os017690_01
Os017705_01
Os017771_01
Os017820_01
Os017851_01
Os017877_01
Os017967_01
Os018002_01
Os018049_01
Os045930_01
Os046039_01
Os046259_01
Os018147_01
Os018213_01
Os018218_01
Os018230_01
Os018246_01
Os018278_01
Os018279_01
Os018289_01
Os018314_01
Os018319_01
Os018329_01
Os018334_01
Os018337_01
Os018375_01
Os018378_01
Os018384_01
Os018393_01
Os018406_01
Os018473_01
Os018475_01
Os018476_01
Os018499_01
Os018503_01
Os018567_01
Os018648_01
Os018656_01
Os018687_01
Os018693_01
Os018709_01
Os018710_01
Os018714_01
Os018730_01
Os018766_01
Os018794_01
Os018797_01
Os052101_01
Os052118_01
Os052121_01
Os018832_01
Os018837_01
Os018949_01
Os019049_01
Os019056_01
Os019066_01
Os019068_01
Os019111_01
Os019197_01
Os019207_01
Os019514_01
Os019551_01
Os019580_01
Os019597_01
Os019636_01
Os019649_01
Os019681_01
Os019702_01
Os019724_01
Os019860_01
Os019891_01
Os019925_01
Os019985_01
Os020043_01
Os020145_01
Os020146_01
Os020249_01
Os020250_01
Os020270_01
Os020310_01
Os020336_01
Os020481_01
Os020529_01
Os020576_01
Os020607_01
Os052788_01
Os052844_01
Os053062_01
Os020623_01
Os020661_01
Os020667_01
Os020707_01
Os020744_01
Os020802_01
Os020896_01
Os020903_01
Os020952_01
Os020962_01
Os020991_01
Os021048_01
Os021051_01
Os021093_01
Os021112_01
Os021114_01
Os021155_01
Os021165_01
Os021216_01
Os021292_01
Os021331_01
Os021367_01
Os021440_01
Os021490_01
Os021497_01
Os021728_01
Os021804_01
Os021823_01
Os021846_01
Os021884_01
Os021890_01
Os021900_01
Os021927_01
Os021942_01
Os021954_01
Os054576_01
Os054597_01
Os054607_01
Os022045_01
Os022055_01
Os022058_01
Os022061_01
Os022089_01
Os022098_01
Os022133_01
Os022139_01
Os022157_01
Os022247_01
Os022411_01
Os022421_01
Os022703_01
Os023056_01
Os023212_01
Os023269_01
Os023487_01
Os023818_01
Os023872_01
Os024218_01
Os024378_01
Os024716_01
Os024773_01
Os024774_01
Os024813_01
Os024838_01
Os024883_01
Os024959_01
Os024975_01
Os025232_01
Os025844_01
Os025874_01
Os025931_01
Os026035_01
Os026464_01
Os055644_01
Os055766_02
Os056012_01
Os026917_01
Os026996_01
Os027379_02
Os027608_01
Os027615_01
Os027659_01
Os027680_01
Os027681_01
Os027799_01
Os027989_01
Os028229_01
Os029072_01
Os029513_01
Os029514_01
Os029620_01
Os029734_01
Os029873_01
Os029876_01
Os029929_01
Os029980_01
Os029984_01
Os030066_01
Os030125_01
Os030128_01
Os030245_01
Os030343_01
Os030354_01
Os030407_01
Os030451_01
Os030618_01
Os030928_01
Os031090_01
Os031094_01
Os031095_01
Os031145_01
Os031591_01
Os031596_01
Os031650_01
Os031712_01
Os031851_01
Os031927_01
Os031993_01
Os032009_01
Os032244_01
Os032245_01
Os032356_01
Os032360_01
Os032369_01
Os032497_01
Os032687_01
Os032731_01
Os033917_01
Os033975_01
Os034414_01
Os034520_01
Os034644_01
Os034869_01
Os034944_01
Os035020_01
Os035048_01
Os035709_01
Os035712_01
Os035905_01
Os036152_01
Os036329_01
Os036380_01
Os036576_01
Os036618_01
Os037447_01
Os038126_01
Os038136_01
Os038486_01
Os039547_01
Os039646_01
Os039715_01
Os039861_01
Os040248_01
Os040311_01
Os041060_01
Os041281_01
Os041762_01
Os041926_01
Os042038_01
Os042108_01
Os042228_01
Os042306_01
Os042560_01
Os042804_01
Os043204_01
Os043270_01
Os043332_01
Os043398_01
Os043722_01
Os043894_01
Os044731_01
Os045256_01
Os045518_01
Os045565_01
Os045600_01
Os045654_01
Os045677_01
Os046402_01
Os046493_01
Os046705_01
Os046795_01
Os046825_01
Os046919_01
Os047236_01
Os047638_01
Os047641_01
Os047671_01
Os047974_01
Os048254_01
Os049100_01
Os051051_01
Os051905_01
Os051916_01
Os051951_01
Os051956_01
Os051966_01
Os051970_01
Os051973_01
Os051998_01
Os052009_01
Os052018_01
Os052028_01
Os052032_02
Os052033_01
Os052050_01
Os052059_01
Os052069_01
Os052093_01
Os052095_01
Os052096_01
Os052123_01
Os052151_01
Os052154_01
Os052184_01
Os052185_01
Os052198_01
Os052215_01
Os052217_01
Os052234_01
Os052236_01
Os052257_01
Os052266_01
Os052270_01
Os052291_01
Os052327_01
Os052337_01
Os052350_01
Os052360_01
Os052411_01
Os052430_01
Os052436_01
Os052461_01
Os052508_01
Os052515_01
Os052557_01
Os052620_01
Os052626_01
Os052672_01
Os052688_01
Os052693_01
Os052715_01
Os052717_01
Os052724_01
Os053063_01
Os053097_01
Os053145_01
Os053207_01
Os053208_01
Os053343_01
Os053523_01
Os053711_01
Os053847_01
Os053922_01
Os053990_01
Os054037_01
Os054048_01
Os054142_01
Os054163_01
Os054316_01
Os054382_01
Os054392_01
Os054419_01
Os054432_01
Os054447_01
Os054458_01
Os054468_01
Os054491_01
Os054492_01
Os054493_01
Os054499_01
Os054507_01
Os054509_01
Os054538_01
Os054550_01
Os054556_01
Os054559_01
Os054627_01
Os054646_01
Os054660_01
Os054674_01
Os054677_01
Os054681_01
Os054687_01
Os054699_01
Os054700_01
Os054705_01
Os054752_01
Os054753_01
Os054774_01
Os054789_01
Os054815_01
Os054883_01
Os054900_01
Os055024_01
Os055030_01
Os055050_01
Os055053_01
Os055085_01
Os055103_01
Os055125_01
Os055155_01
Os055156_01
Os055188_01
Os055294_01
Os055302_01
Os055517_01
Os055529_01
Os055567_01
Os055624_01
Os056140_01
Os056174_01
Os056390_01
Os056527_01
Os056622_02
Os056851_01
Os056903_02
Os056918_02
Os056919_02
Os057111_02
Os057146_02
Os057158_02
Os057161_02
Os057176_02
Os057198_02
Os057257_02
Os057361_02
Os057460_02
Os057595_02
Os057633_02

SUPPLEMENTAL TABLE 14
Oligo ID

Os000014_01
Os000236_01
Os000447_01
Os000532_01
Os000654_01
Os000757_01
Os000942_01
Os001081_01
Os001087_01
Os001320_01
Os001497_01
Os001900_01
Os001903_01
Os001911_01
Os001920_01
Os001941_01
Os002028_01
Os002063_01
Os002358_01
Os002390_01
Os002698_01
Os003026_01
Os003284_01
Os003332_01
Os003410_02
Os003719_01
Os003880_01
Os004740_01
Os004928_01
Os006465_01
Os007544_01
Os007764_01
Os008159_01
Os032197_01
Os032601_01
Os008548_01
Os008629_01
Os008639_01
Os008761_01
Os008777_01
Os009032_01
Os009047_01
Os009164_01
Os009211_01
Os009365_01
Os009373_01
Os009744_01
Os009821_01
Os010171_01
Os010621_01
Os010685_01
Os010789_01
Os010863_01
Os010883_01
Os010904_01
Os010941_01
Os010968_01
Os011001_01
Os011018_01
Os011249_01
Os011340_01
Os011359_01
Os011414_01
Os011787_01
Os011870_01
Os011919_01
Os011920_01
Os011972_01
Os053057_01
Os053249_01
Os012049_01
Os012062_01
Os012193_01
Os012444_01
Os012479_01
Os012497_01
Os012520_01
Os012619_01
Os012632_01
Os012731_01
Os012777_01
Os012900_01
Os012955_01
Os013036_01
Os013103_01
Os013114_01
Os013186_01
Os013192_01
Os013812_01
Os013849_01
Os013858_01
Os013877_01
Os014441_01
Os014442_01
Os014512_01
Os014535_01
Os014604_01
Os014773_01
Os014803_01
Os014927_01
Os014930_01
Os014944_01
Os015168_01
Os015317_01
Os015599_01
Os015790_01
Os015868_01
Os015880_01
Os015886_01
Os015961_01
Os015991_01
Os015998_01
Os016030_01
Os016243_01
Os016461_01
Os016486_01
Os016505_01
Os016536_01
Os016744_01
Os016826_01
Os016842_01
Os016929_01
Os016940_01
Os017163_01
Os017259_01
Os017289_01
Os017292_01
Os017387_01
Os017443_01
Os017526_01
Os017806_01
Os017833_01
Os017879_01
Os017893_01
Os018042_01
Os018056_01
Os018065_01
Os018188_01
Os018211_01
Os018286_01
Os018327_01
Os018341_01
Os018381_01
Os018411_01
Os018734_01
Os018786_01
Os018863_01
Os018880_01
Os019350_01
Os019547_01
Os019961_01
Os020073_01
Os020121_01
Os020130_01
Os020215_01
Os020216_01
Os020360_01
Os020769_01
Os020838_01
Os020903_01
Os020908_01
Os021018_01
Os021231_01
Os021293_01
Os021523_01
Os021547_01
Os021603_01
Os021643_01
Os021699_01
Os021735_01
Os021920_01
Os022004_01
Os022097_01
Os022116_01
Os022130_01
Os022167_01
Os022272_01
Os022365_01
Os022389_01
Os022404_01
Os022433_01
Os022486_01
Os022584_01
Os022844_01
Os022866_01
Os022934_01
Os022949_01
Os023044_01
Os023116_01
Os023426_01
Os023465_01
Os023540_01
Os023546_01
Os023575_01
Os023615_01
Os023620_01
Os023625_01
Os023706_01
Os023709_01
Os023781_01
Os023849_01
Os023903_01
Os024106_01
Os024220_01
Os024292_01
Os024380_01
Os024445_01
Os024474_01
Os024549_01
Os024563_01
Os024580_01
Os025848_01
Os026060_01
Os026332_01
Os026407_01
Os026674_01
Os027703_01
Os027970_01
Os028746_01
Os029007_01
Os029131_01
Os029207_01
Os029213_01
Os030104_01
Os030170_01
Os030451_01
Os030533_01
Os030831_01
Os030928_01
Os031305_01
Os031471_01
Os031729_01
Os031778_01
Os031802_01
Os032027_01
Os033011_01
Os033293_01
Os033917_01
Os034833_01
Os035629_01
Os038059_01
Os038174_01
Os038423_01
Os038428_01
Os038610_01
Os038788_01
Os038797_01
Os039193_01
Os039878_01
Os040314_01
Os040381_01
Os040673_01
Os042100_01
Os043037_01
Os043064_01
Os045418_01
Os048106_01
Os049451_01
Os050390_01
Os050416_01
Os051966_01
Os051974_01
Os052000_01
Os052221_01
Os052225_01
Os052361_01
Os052557_01
Os052669_01
Os052782_02
Os053731_02
Os053954_01
Os054377_01
Os054428_01
Os054564_01
Os054610_01
Os054621_01
Os054637_01
Os054809_01
Os055421_01
Os055470_01
Os055638_01

SUPPLEMENTAL TABLE 15
Oligo ID

Os000325_01
Os000431_01
Os000438_01
Os000561_01
Os000842_01
Os000851_01
Os000963_01
Os001094_01
Os001268_01
Os001355_01
Os001400_01
Os001428_01
Os001667_01
Os001878_01
Os001903_01
Os001927_01
Os001957_01
Os001958_01
Os002088_01
Os002092_01
Os002160_01
Os002444_01
Os003284_01
Os003542_02
Os003687_01
Os004160_01
Os007263_01
Os008400_01
Os008424_01
Os008476_01
Os008515_01
Os008525_01
Os008979_01
Os009046_01
Os009237_01
Os009255_01
Os009739_01
Os009835_01
Os009883_01
Os010067_01
Os010191_01
Os010339_01
Os010372_01
Os012623_01
Os012723_01
Os012844_01
Os013059_01
Os013159_01
Os013389_01
Os013462_01
Os013908_01
Os013945_01
Os014001_01
Os014505_01
Os014639_01
Os014740_01
Os015017_01
Os015069_01
Os016135_01
Os016615_01
Os016882_01
Os017025_01
Os017362_01
Os017416_01
Os017420_01
Os017972_01
Os018729_01
Os019066_01
Os019289_01
Os019580_01
Os020167_01
Os020411_01
Os021165_01
Os021494_01
Os021735_01
Os021880_01
Os022128_01
Os022135_01
Os022421_01
Os022742_01
Os022803_01
Os023013_01
Os023116_01
Os024774_01
Os024971_01
Os026917_01
Os027596_01
Os027650_01
Os027666_01
Os027740_01
Os027913_01
Os029232_01
Os029410_01
Os029446_01
Os029727_01
Os029761_01
Os029853_01
Os029876_01
Os029900_01
Os030007_01
Os030071_01
Os030128_01
Os030164_01
Os030170_01
Os030343_01
Os030354_01
Os030451_01
Os030533_01
Os030572_01
Os030742_01
Os030757_01
Os030831_01
Os030928_01
Os030952_01
Os030984_01
Os031525_01
Os031591_01
Os031620_01
Os031666_01
Os031896_01
Os031927_01
Os032009_01
Os032021_01
Os032244_01
Os032472_01
Os032815_01
Os032997_01
Os033005_01
Os033022_01
Os033246_01
Os033520_01
Os033917_01
Os033970_01
Os034222_01
Os034350_01
Os035155_01
Os035299_01
Os035537_01
Os035558_01
Os035559_01
Os035629_01
Os035648_01
Os035696_01
Os036076_01
Os036093_01
Os036294_01
Os036618_01
Os037046_01
Os037141_01
Os037254_01
Os038126_01
Os038428_01
Os038551_01
Os038894_01
Os039193_01
Os039478_01
Os039740_01
Os039903_01
Os040381_01
Os040600_01
Os041060_01
Os042132_01
Os043076_01
Os043662_01
Os043722_01
Os043736_01
Os044197_01
Os044205_01
Os044300_01
Os045461_01
Os045518_01
Os046239_01
Os047642_01
Os047671_01
Os047874_01
Os048512_01
Os049971_01
Os051887_01
Os051892_01
Os051896_01
Os051930_01
Os051949_01
Os051956_01
Os051959_01
Os051981_01
Os051998_01
Os052018_01
Os052032_02
Os052085_01
Os052118_01
Os052131_01
Os052240_01
Os052258_01
Os052303_01
Os052557_01
Os052669_01
Os052782_02
Os053842_01
Os054370_01
Os054371_01
Os054376_01
Os054379_01
Os054419_01
Os054421_01
Os054428_01
Os054431_01
Os054459_01
Os054468_01
Os054495_01
Os054496_01
Os054522_01
Os054570_01
Os054571_01
Os054640_01
Os054645_01
Os055155_01
Os055156_01
Os055681_01
Os056527_01
Os056910_02
Os056928_02
Os057254_02
Os057480_02
Os057570_02

SUPPLEMENTAL TABLE 16

| Oligo ID | Annotation |
|---|---|
| Os000251_01 | AP2 domain, putative |
| Os000389_01 | WRKY DNA-binding protein |
| Os000417_01 | GATA zinc finger, putative |
| Os000545_01 | No apical meristem (NAM) protein, putative |
| Os000691_01 | PHD zinc finger protein, putative |
| Os000997_01 | Myb-like DNA-binding domain, putative |
| Os001497_01 | putative Cys2/His2 zinc-finger protein |
| Os001578_01 | Zinc finger C-x8-C-x5-C-x3-H type (and similar), putative |
| Os001621_01 | Zinc finger, C3HC4 type (RING finger), putative |
| Os002732_01 | myb-like DNA-binding domain, SHAQKYF class, putative |
| Os002858_01 | ZF-HD homeobox protein |
| Os003223_01 | MYB-related protein, putative |
| Os003326_01 | Similar to zinc finger transcription factor WRKY1 |
| Os004298_01 | myb-like DNA-binding domain, SHAQKYF class, putative |
| Os004372_01 | Zinc finger, C3HC4 type (RING finger), putative |
| Os004865_01 | Helix-loop-helix DNA-binding domain, putative |
| Os005810_01 | Zinc finger, C2H2 type, putative |
| Os005941_01 | Helix-loop-helix DNA-binding domain, putative |
| Os005944_01 | bHLH transcription factor bHLH091, putative |
| Os005987_01 | Zinc finger, C3HC4 type (RING finger), putative |
| Os007216_01 | B3 DNA binding domain, putative |
| Os008571_01 | GRAS family transcription factor, putative |
| Os008742_01 | B-box zinc finger, putative |
| Os009133_01 | C2H2-type zinc finger protein ZFP36 |
| Os009161_01 | CCAAT-binding transcription factor (CBF-B/NF-YA) subunit B, putative |
| Os009247_01 | Homeobox associated leucine zipper, putative |
| Os009459_01 | AP2 domain, putative |
| Os010313_01 | AP2 domain, putative |
| Os010599_01 | Dof domain, zinc finger, putative |
| Os010664_01 | No apical meristem (NAM) protein, putative |
| Os010835_01 | NAC domain protein NAC1 |
| Os010863_01 | Homeobox domain, putative |
| Os010878_01 | AN1-like Zinc finger, putative |
| Os010961_01 | Zinc finger C-x8-C-x5-C-x3-H type (and similar), putative |
| Os011169_01 | TCP family transcription factor, putative |
| Os011195_01 | HSF-type DNA-binding domain, putative |
| Os011854_01 | probable zinc finger protein [imported] - Arabidopsis thaliana |
| Os011930_01 | Zinc finger |
| Os012037_01 | G-box binding factor 1 - maize |
| Os012160_01 | PHD-finger, putative |
| Os012216_01 | bHLH transcription factor PTF1 |
| Os012444_01 | No apical meristem (NAM) protein, putative |
| Os012506_01 | Similar to Pspzf zinc finger protein |
| Os012683_01 | HSF-type DNA-binding domain, putative |
| Os012777_01 | NF-X1 type zinc finger, putative |
| Os012785_01 | Zinc finger, C3HC4 type (RING finger), putative |
| Os012945_01 | putative AP2 domain transcription factor |
| Os013151_01 | Dof domain, zinc finger, putative |
| Os013315_01 | Zinc finger, C3HC4 type (RING finger), putative |
| Os013432_01 | AP2 domain, putative |
| Os013518_01 | TRAF-type zinc finger, putative |
| Os013609_01 | putative zinc finger protein |
| Os013659_01 | putative NAC-domain protein |
| Os013718_01 | Zinc finger, C3HC4 type (RING finger), putative |
| Os013791_01 | Zinc finger C-x8-C-x5-C-x3-H type (and similar), putative |
| Os013810_01 | bZIP transcription factor, putative |
| Os013812_01 | No apical meristem (NAM) protein, putative |
| Os013844_01 | Myb-like DNA-binding domain, putative |
| Os013885_01 | Helix-loop-helix DNA-binding domain, putative |
| Os013929_01 | Zinc finger, C3HC4 type (RING finger), putative |
| Os014040_01 | Helix-loop-helix DNA-binding domain, putative |
| Os014060_01 | Helix-loop-helix DNA-binding domain, putative |
| Os014104_01 | DRE binding factor 1 |
| Os014511_01 | putative Cys2/His2 zinc-finger protein |
| Os014634_01 | Zinc finger, C3HC4 type (RING finger), putative |
| Os014704_01 | SRF-type transcription factor (DNA-binding and dimerisation domain), |
| Os014803_01 | No apical meristem (NAM) protein, putative |
| Os014810_01 | GRAS family transcription factor, putative |
| Os015150_01 | AN1-like Zinc finger, putative |
| Os015245_01 | putative CCAAT-binding transcription factor |
| Os015612_01 | putative calmodulin-binding transcription factor |
| Os015694_01 | Helix-loop-helix DNA-binding domain, putative |
| Os015957_01 | probable RING zinc finger protein [imported] - Arabidopsis thaliana |
| Os015985_01 | Zinc finger C-x8-C-x5-C-x3-H type (and similar), putative |
| Os015997_01 | contains similarity to MYB-related DNA-binding protein |
| Os016074_01 | bHLH protein, putative |
| Os016285_01 | No apical meristem (NAM) protein, putative |
| Os016373_01 | Zinc finger, C3HC4 type (RING finger), putative |
| Os016599_01 | putative helix-loop-helix DNA-binding protein |
| Os016793_01 | bZIP transcription factor, putative |
| Os016888_01 | Similar to zinc-finger protein S3574 [imported] - rice |
| Os017165_01 | HSF-type DNA-binding domain, putative |
| Os017664_01 | Zinc finger, C3HC4 type (RING finger), putative |
| Os017707_01 | Zinc finger, C3HC4 type (RING finger), putative |
| Os018013_01 | Zinc finger, C3HC4 type (RING finger), putative |
| Os018078_01 | No apical meristem (NAM) protein, putative |
| Os018111_01 | Zinc finger C-x8-C-x5-C-x3-H type (and similar), putative |
| Os018253_01 | Myb-like DNA-binding domain, putative |
| Os018341_01 | Zinc finger, C3HC4 type (RING finger), putative |
| Os018795_01 | Helix-loop-helix DNA-binding domain, putative |
| Os018811_01 | Helix-loop-helix DNA-binding domain, putative |
| Os019506_01 | similar to an Arabidopsis thaliana C3HC4-type zinc finger |
| Os019547_01 | myb-like DNA-binding domain, SHAQKYF class, putative |
| Os019756_01 | AP2 domain, putative |
| Os019775_01 | Homeobox domain, putative |
| Os019855_01 | Similar to probable wrky transcription factor 24 (wrky dna-binding protein 24) |
| Os019879_01 | CCAAT-binding transcription factor (CBF-B/NF-YA) subunit B, putative |
| Os019891_01 | HSF-type DNA-binding domain, putative |
| Os020073_01 | AP2 domain, putative |
| Os020290_01 | Helix-loop-helix DNA-binding domain, putative |
| Os020660_01 | No apical meristem (NAM) protein, putative |
| Os021488_01 | CHY zinc finger, putative |
| Os021539_01 | CCAAT-box binding factor HAP5 homolog |
| Os021770_01 | PHD-finger, putative |
| Os021893_01 | DRE-binding protein 1A |
| Os021980_01 | zinc finger transcription factor-like protein |
| Os022001_01 | Myb-like DNA-binding domain, putative |
| Os022217_01 | WRKY DNA-binding domain, putative |
| Os022261_01 | Helix-loop-helix DNA-binding domain, putative |
| Os022309_01 | Zinc finger C-x8-C-x5-C-x3-H type (and similar), putative |
| Os022407_01 | AP2 domain, putative |
| Os022789_01 | myb-like DNA-binding domain, SHAQKYF class, putative |
| Os022837_01 | bZIP transcription factor, putative |
| Os022926_01 | Myb-like DNA-binding domain, putative |
| Os022936_01 | AP2 domain, putative |
| Os022939_01 | bZIP transcription factor, putative |
| Os022981_01 | WRKY DNA-binding domain, putative |
| Os022994_01 | transcription factor, putative |
| Os023048_01 | Similar to NAM-like protein, 59502-58357 [imported] - Arabidopsis thaliana |
| Os023247_01 | Dof domain, zinc finger, putative |
| Os023361_01 | Zinc finger C-x8-C-x5-C-x3-H type (and similar), putative |
| Os023582_01 | Zinc finger, C3HC4 type (RING finger), putative |
| Os023706_01 | OsNAC1 protein |
| Os023722_01 | Similar to probable C2H2-type zinc finger protein [imported] - Arabidopsis thaliana |
| Os023766_01 | Zinc finger, C3HC4 type (RING finger), putative |
| Os023819_01 | AP2 domain, putative |
| Os023851_01 | Zinc finger C-x8-C-x5-C-x3-H type (and similar), putative |
| Os024014_01 | No apical meristem (NAM) protein, putative |
| Os024044_01 | Zinc finger, C3HC4 type (RING finger), putative |
| Os024139_01 | Helix-loop-helix DNA-binding domain, putative |
| Os024185_01 | WRKY DNA-binding domain, putative |
| Os024260_01 | WRKY DNA-binding domain, putative |
| Os024331_01 | AP2 domain-containing protein Rap211 |
| Os024347_01 | Zinc finger, C3HC4 type (RING finger), putative |
| Os024354_01 | probable wrky transcription factor 75 (wrky dna-binding protein 75) |
| Os024855_01 | Zinc finger, C3HC4 type (RING finger), putative |
| Os024909_01 | WRKY DNA-binding domain, putative |
| Os025741_01 | Similar to DNA-binding protein WRKY3 |
| Os025760_01 | No apical meristem (NAM) protein, putative |
| Os025804_01 | Zinc finger C-x8-C-x5-C-x3-H type (and similar), putative |
| Os027631_01 | Myb-like DNA-binding domain, putative |
| Os028213_01 | putative No apical meristem (NAM) protein |
| Os028563_01 | WRKY DNA-binding domain, putative |
| Os030024_01 | Myb-like DNA-binding domain, putative |
| Os031345_01 | AP2 domain, putative |
| Os031898_01 | WRKY DNA-binding domain, putative |
| Os032039_01 | myb-like DNA-binding domain, SHAQKYF class, putative |
| Os032815_01 | AP2 domain containing protein RAP2.11, putative |
| Os032860_01 | Myb-like DNA-binding domain, putative |
| Os034685_01 | AP2 domain, putative |
| Os035258_01 | bZIP transcription factor, putative |
| Os037212_01 | bZIP transcription factor, putative |
| Os037266_01 | myb-like DNA-binding domain, SHAQKYF class, putative |
| Os038466_01 | B3 DNA binding domain, putative |
| Os039501_01 | AP2 domain, putative |
| Os040595_01 | Helix-loop-helix DNA-binding domain, putative |
| Os044033_01 | Zinc finger, C2H2 type, putative |
| Os046901_01 | Helix-loop-helix DNA-binding domain, putative |
| Os048239_01 | AP2 domain, putativ |
| Os048604_01 | Helix-loop-helix DNA-binding domain, putative |
| Os050111_01 | AP2 domain, putative |
| Os051051_01 | bZIP transcription factor, putative |
| Os051257_01 | Zinc finger, C3HC4 type (RING finger), putative |
| Os052000_01 | No apical meristem (NAM) protein, putative |
| Os052001_01 | OsNAC5 protein [imported] - rice |
| Os052160_01 | AP2 domain, putative |
| Os052206_01 | GRAS family transcription factor, putative |
| Os052686_01 | Dof domain, zinc finger, putative |
| Os053145_01 | AN1-like Zinc finger, putative |
| Os054564_01 | CHY zinc finger, putative |
| Os054673_01 | zinc finger transcription factor ZF1 |
| Os054835_01 | zinc-finger protein S3574 [imported] - rice |
| Os054879_01 | Myb-like DNA-binding domain, putative |
| Os054889_01 | Zinc finger, C3HC4 type (RING finger), putative |
| Os054901_01 | Zinc finger C-x8-C-x5-C-x3-H type (and similar), putative |
| Os054996_01 | myb protein homolog - ric |
| Os055029_01 | WRKY DNA-binding domain, putative |
| Os055470_01 | Homeobox domain, putative |
| Os055619_01 | WRKY DNA-binding domain, putative |
| Os055630_01 | AP2 domain, putative |
| Os055677_01 | Zinc finger, C2H2 type, putative |
| Os055974_01 | Myb-like DNA-binding domain, putative |
| Os056374_01 | WRKY DNA-binding domain, putative |
| Os056731_01 | myb-like DNA-binding domain, SHAQKYF class, putative |
| Os057327_02 | myb factor |
| Os057402_02 | HSF-type DNA-binding domain, putative |

SUPPLEMENTAL TABLE 17

| | |
|---|---|
| Primer name | Oligo Sequence |

| | |
|---|---|
| 0s017777_F | GAATCGGAATCGCGT\|ATTGC |
| 0s017777_R | GATTCGCTTTCGTCGCTCGATG |
| 0s013814_F | GAAGTGCAGGTAAAGCTTGC |
| 0s013814_R | CATGTGCAAGACGCGACTTGTTC |
| 0s022866_F | CGATGATGGGGCCGAACGTGTCG |
| 0s022866_R | CATGCCGGCGTTGAAGTAGAGCGG |
| 0s018470_F | GGATGAACTCCATGCCGTCACC |
| 0s018470_R | CGATCCTATACACTTCTCTACTG |
| 0s052146_F | CGAATCTTGGTTGCTGCGAC |
| 0s052146_R | GTTGTTCTGCAGATACAGTGTTG |
| 0s001377_F | GAGGATCATGACAGAGGTGGC |
| 0s001377_R | CAATCCCGTGGCGTGCCCAC |
| 0s023774_F | GATGTTTTATGAGGGATCAGATC |
| 0s023774_R | GATAGTTAGGTACTTTCACAACC |
| 0s010867_F | GCAGCCAGAAGAAGCTAGCGA |
| 0s010867_R | GGCACCTGCATGTCCGCACGC |

What is claimed is:

1. A drought induced rice promoter comprising a sequence listing of SEQ ID NO: 1.

2. A drought induced rice promoter comprising a sequence listing of SEQ ID NO: 2.

3. A drought induced rice promoter comprising a sequence listing of SEQ ID NO: 3.

4. A drought induced rice promoter comprising a sequence listing of SEQ ID NO: 4.

5. A drought induced rice promoter comprising a sequence listing of SEQ ID NO: 5.

6. A drought induced rice promoter comprising a sequence listing of SEQ ID NO: 6.

7. A drought induced rice promoter comprising a sequence listing of SEQ ID NO: 7.

8. A drought induced rice promoter comprising a sequence listing of SEQ ID NO: 8.

9. A drought induced rice promoter comprising a sequence listing of SEQ ID NO: 9.

10. A drought induced rice promoter comprising a sequence listing of SEQ ID NO: 10.

11. A drought induced rice promoter comprising a sequence listing of SEQ ID NO: 11.
